SEQUENCE LISTING (25 sequences)

SEQ ID NO: 1 - DNA, 97 bp, Homo sapiens
tgctgttgacagtgagcgcaggaagaattgttttaggatatagtgaagccacagatgtatatcctaaaacaattcttcctttgcctactgcctcgga

SEQ ID NO: 2 - DNA, 97 bp, Homo sapiens
tgctgttgacagtgagcgccccacaagatgttattaataatagtgaagccacagatgtattattaataacatcttgtgggatgcctactgcctcgga

SEQ ID NO: 3 - DNA, 96 bp, Homo sapiens
tgctgttgacagtgagcgaggcagaagtatgcaaagcattagtgaagccacagatgtaatgctttgcatacttctgcctgtgcctactgcctcgga

SEQ ID NO: 4 - DNA, 19 bp, Homo sapiens
gatgggcggcggaaaatag

SEQ ID NO: 5 - DNA, 21 bp, Homo sapiens
gcgtggattctgcataatggt

SEQ ID NO: 6 - DNA, 21 bp, Homo sapiens
ctcgccagtgaaatgatggct

SEQ ID NO: 7 - DNA, 21 bp, Homo sapiens
gtcggagattcgtagctggat

SEQ ID NO: 8 - DNA, 21 bp, Homo sapiens
gacgcctttatcctctctgcg

SEQ ID NO: 9 - DNA, 23 bp, Homo sapiens
acgacctgatccaattctgccca

SEQ ID NO: 10 - DNA, 21 bp, Homo sapiens
ctgcctgggtttccatcttct

SEQ ID NO: 11 - DNA, 23 bp, Homo sapiens
gccattcatgtcagagctacact

SEQ ID NO: 12 - DNA, 22 bp, Homo sapiens
catcaatgacaaggtggccaag

SEQ ID NO: 13 - DNA, 20 bp, Homo sapiens
gggcttgatggggtgatagg

SEQ ID NO: 14 - DNA, 20 bp, Homo sapiens
acccccatgactccagagaa

SEQ ID NO: 15 - DNA, 21 bp, Homo sapiens
cttgtgcctgcaaaatcgcat

SEQ ID NO: 16 - DNA, 22 bp, Homo sapiens
caccctctagcaatgtcaactc

SEQ ID NO: 17 - DNA, 21 bp, Homo sapiens
cagactcgggatactctctcc

SEQ ID NO: 18 - DNA, 26 bp, Homo sapiens
gctaacctttgatgctataactacga

SEQ ID NO: 19 - DNA, 21 bp, Homo sapiens
tttgtgcgcatgtagaatctg

SEQ ID NO: 20 - DNA, 20 bp, Homo sapiens
ccctcctcaggtggtgtttg

SEQ ID NO: 21 - DNA, 20 bp, Homo sapiens
cgacaaatgaagcagcgtgt

SEQ ID NO: 22 - DNA, 21 bp, Homo sapiens
ttccacgatatggatttacgg

SEQ ID NO: 23 - DNA, 21 bp, Homo sapiens
gtttcctgttgcattgagtcc

SEQ ID NO: 24 - DNA, 21 bp, Homo sapiens
ctgaccgcagagtcttttcct

SEQ ID NO: 25 - DNA, 20 bp, Homo sapiens
gagtggtttggctggggtaa

(19) United States
(12) Patent Application Publication
(10) Pub. No.: US 2022/0195533 A1
(43) Pub. Date: Jun. 23, 2022

(54) ARYL HYDROCARBON RECEPTOR (AHR) ACTIVATION SIGNATURE AND METHODS FOR DETERMINING AHR SIGNALING STATUS

(71) Applicant: DEUTSCHES KREBSFORSCHUNGSZENTRUM STIFTUNG DES OFFENTLICHEN RECHTS, Heidelberg (DE)

(72) Inventors: Christiane A. OPITZ, Weinheim (DE); Ahmed SADIK, Eppelheim (DE); Saskia TRUMP, Berlin (DE); Soumya R. MOHAPATRA, Dossenheim (DE); Sascha SCHÄUBLE, Jena (DE); Erik FÄSSLER, Burgdorf (DE); Luis F. SOMARRIBAS PATTERSON, Heredia (CR)

(73) Assignee: DEUTSCHES KREBSFORSCHUNGSZENTRUM STIFTUNG DES OFFENTLICHEN RECHTS, Heidelberg (DE)

(21) Appl. No.: 17/599,681

(22) PCT Filed: Mar. 28, 2020
(86) PCT No.: PCT/IB2020/000236

§ 371 Date: Sep. 29, 2021

(30) Foreign Application Priority Data
Mar. 29, 2019 (EP) 19166374.9

(51) Int. Cl. C12Q 1/6886; G16B 25/10; G16B 40/20; G16B 40/30

(52) U.S. Cl. CPC C12Q 1/6886; G16B 25/10; G16B 40/20; C12Q 2600/158; C12Q 2600/112; C12Q 2600/142; C12Q 2600/106; G16B 40/30

(57) ABSTRACT

The present disclosure relates to the generation and uses of an improved set of biomarkers that are aryl hydrocarbon receptor (AHR) target genes, designated as “AHR biomarkers.” The AHR biomarkers described herein allow one to efficiently determine AHR activation groups and sub-groups, in particular for an improved classification of tumors. As used herein, AHR activation groups are called “AHR activation signatures”. The AHR biomarkers comprise markers that are important in diagnosis and therapy, for example for selecting patients for treatment with AHR activation modulating interventions, and monitoring of therapy response.

### CROSS REFERENCE TO RELATED APPLICATION

This application claims the benefit of priority from European Provisional Application No. EP19166374, filed Mar. 29, 2019, the entire contents of which are incorporated herein by reference.

### INCORPORATION BY REFERENCE OF SEQUENCE LISTING

The Sequence Listing in an ASCII text file, named as 38272PCT_SequenceListing.txt of 495 KB, created on Mar. 27, 2020, and submitted to the United States Patent and Trademark Office via EFS-Web, is incorporated herein by reference.

### BACKGROUND

The aryl hydrocarbon receptor (AHR) is a ligand-activated transcription factor involved in the regulation of diverse processes such as embryogenesis, vasculogenesis, drug metabolism, cell motility and immune modulation, and cancer. In preclinical studies AHR activation by tryptophan metabolites generated through indoleamine-2,3-dioxygenase (IDO1) and/or tryptophan-2,3-dioxygenase (TDO2) promoted tumor progression by enhancing the motility, anoikis and clonogenic survival of the tumor cells as well as by suppressing anti-tumor immune responses.

As ligand binding is necessary for AHR activation, the expression level of AHR alone does not allow inference of its activation state. AHR activation is commonly detected by its nuclear translocation, the activity of cytochrome P-450 enzymes or the binding of AHR-ARNT to dioxin-responsive elements (DRE) using reporter assays. While all of these methods are applicable in vitro, they are laborious, require special equipment and are expensive. In addition, relying on cytochrome P-450 enzymes is limited to conditions where they are regulated, which is not always the case, given the ligand and cell type specificity of AHR activation.

AHR target gene expression is context-specific, and therefore an AHR activation signature consisting of diverse AHR target genes is required to efficiently detect AHR activation across different cells/tissues and in response to diverse AHR ligands.

The expression of a specific gene is mostly not regulated by a single transcription factor but several transcriptions factors acting separately or in combination. Therefore a single marker is not specific as a readout for a certain transcription factor. Specific for detecting biomarkers for AHR activity, as we know that AHR target gene expression is very cell type and context dependent a single marker might be a readout for AHR activity in one condition but not the other. In addition, a single biomarker cannot capture functional outcomes. (Rothhammer, V. & Quintana, F. J. 2019. The aryl hydrocarbon receptor: an environmental sensor integrating immune responses in health and disease. Nat Rev Immunol, doi:10.1038/s41577-019-0125-8).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. A diagram of the workflow for generating the AHR signature. The graphical representation describes the generation of the AHR signature by integrating results of natural language processing of free full texts and abstracts of PubMed and PubMed-Central, and mined gene expression datasets.

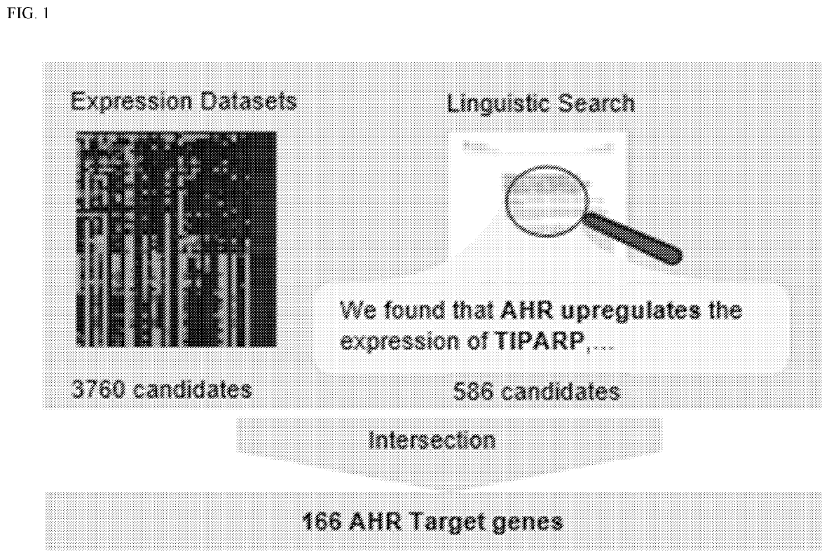

FIG. 2. A circular bar graph representing eight biological processes gene ontology groups that are enriched in the AHR signature genes. The inner most circle represents the color code of each ontology groups. Each bar represents a significantly enriched ontology term. The bars are ordered in a descending order of highest significance in a clockwise fashion. The colors of each bar correspond to the significance of enrichment (log pv=−log 10 p-value of enrichment). The length of each bar and the numbers in the outer circle represent the number of genes from the AHR signature sharing the same ontology term.

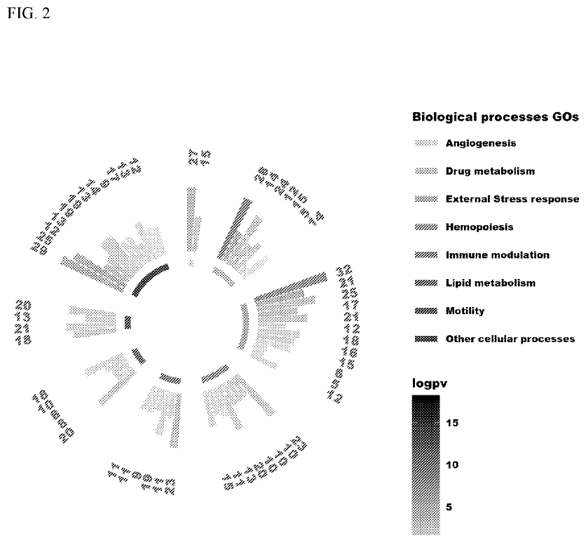

FIGS. 3A-3D. Barcode plots showing the direction of regulation of the AHR signature after performing differential gene regulation of: (A) MCF7 cells exposed to 100 nM TCDD for 24 hours compared to DMSO (GSE98515). (B) A549 cells exposed to 10 nM TCDD for six hours (GSE109576). (C) HepG2 cells exposed to 10 nM TCDD for 24 hours (GSE28878), (D) Human Multipotent Adipose-Derived Stem cells (hMADS) exposed to 25 nM TCDD for 24 hours (GSE32026). The x-axis represents the moderated t-statistic values for all genes in the comparison. The darker grey scales represent the lower and upper quartiles of all the genes. The vertical barcode lines represent the distribution of the AHR signature genes. The worm line representation above the barcode shows the direction of regulation of the AHR signature.

FIGS. 4A-4D. Barcode plots showing the direction of regulation of the AHR signature after performing differential gene regulation of: (A) primary AML cells exposed to 500 nM SR1 for 16 hours (GSE48843), (B) CD34 positive hematopoietic stem cells (HSC) treated with 1 uM SR1 for 7 days (GSE67093), (C) hESC cells treated with SR1 for 24 hours (GSE52158), (D) A549 cells exposed to 10 uM CH223191 for six hours (GSE109576).

FIGS. 5A-5B. Barcode plots showing the direction of regulation of the AHR signature after performing differential gene regulation of: (A) Th17 cells exposed to 200 nM FICZ for 16 hours (GSE102045), (B) U87 cells exposed to 100 nM FICZ for 24 hours.

FIGS. 6A-6C. Barcode plots showing the direction of regulation of the AHR signature after performing differential gene regulation of: (A) U87 cells exposed to 100 uM Kyn for 8 hours (GSE25272), (B) U87 cells exposed to 50 uM KynA for 24 hours, (C) U87 cells exposed to 50 uM I3CA for 24 hours.

FIGS. 7A-7C. (A) mRNA expression of selected AHR target genes in U-87MG-shCtrl (shCtrl) and U-87MG-shAHR (shAHR) treated with 10 nM TCDD or vehicle for 24 h (n=3). (B) mRNA expression of selected AHR target genes in U-87MG-shCtrl and U-87MG-shAHR treated with 100 nM FICZ or vehicle for 24 h (n=4). (C) mRNA expression of selected AHR target genes in U-87 MG-shCtrl and U-87MG-shAHR treated with 50 pM Kyn or vehicle for 24 h (n=3). n values represent the number of independent experiments. Data represented as mean±S.E.M and were analyzed by two-tailed paired student's t-test (e, j). *P<0.05, **P<0.01, ***P<0.001, ****P<0.0001. n.s., not significant. * vehicle compared to treatment: # treatment in shC compared to shAHR

FIG. 8. Pie chart representations showing the results of gene set enrichment using roast on patients of 32 primary TCGA tumors after median separation of the patients into groups of high and low expression of IDO1 or TDO2. The missing pie-charts designate that there was no significant AHR modulation detected in the high-low group comparisons. The darker color shades in the pie charts show the percentage of up-regulated AHR signature genes in the high-low comparisons, and subsequently, the lighter color shades show the percentage of down-regulated AHR signature genes in the high-low comparisons. The sum of the shaded pie chart segments denotes the percentage of AHR signature genes that were differentially regulated.

FIG. 9. Density plots showing multi-modal distributions of the log 2 transcripts per million (log 2 TPM) expression levels of IDO1 (light grey) and TDO2 (dark grey) in 32 primary TCGA tumors. The vertical dotted lines show the median value for IDO1 (light grey) or TDO2 (dark grey).

FIGS. 10A-10B: (A) Circos showing the connections of IDO1 and TDO2 if co-expressed in WGCNA modules, positively associated with AHR activation in Stomach adenocarcinoma (STAD). The circular segments correspond to the WGCNA module, and the connections have the same color as the corresponding module. The size of the module is proportionate to the number of genes. (B) Box plot representation of the AHR activation score in STAD subtypes. Group comparison was performed by a Wilcox-sum rank test.

FIGS. 11A-11B. (A) Circos showing the connections of IDO1 and TDO2 if co-expressed in WGCNA modules, positively associated with AHR activation in Thyroid carcinoma (THCA). The circular segments correspond to the WGCNA module, and the connections have the same color as the corresponding module. The size of the module is proportionate to the number of genes. (B) Box plot representation of the AHR activation score in THCA subtypes. Group comparisons were performed by a Wilcox-sum rank test.

FIGS. 12A-12B. (A) Circos showing the connections of IDO1 and TDO2 if co-expressed in WGCNA modules, positively associated with AHR activation in Glioblastoma multiforme (GBM). The circular segments correspond to the WGCNA module, and the connections have the same color as the corresponding module. The size of the module is proportionate to the number of genes. (B) Box plot representation of the AHR activation score in GBM subtypes. Group comparisons were performed by a Wilcox-sum rank test. SEQ ID Nos 1 to 3 show shAHR sequences for knockdown experiments. SEQ ID Nos 4 to 25 show oligonucleotide sequences for rtPCR experiments.

FIG. 13. Boxplot representation of the expression of IDO1 (left) and TDO2 (right) as log 2 counts per million in the AHR activation subgroups of TCGA-STAD. Wilcoxon sum-rank test was used for the group comparisons.

FIG. 14. Boxplot representation of the expression of IDO1 (left) and TDO2 (right) as log 2 counts per million in the AHR activation subgroups of TCGA-THCA. Wilcoxon ranked summed test was used for the group comparisons.

FIG. 15. Boxplot representation of the expression of IDO1 (left) and TDO2 (right) as log 2 counts per million in the AHR activation subgroups of TCGA-GBM. Wilcoxon ranked summed test was used for the group comparisons.

FIG. 16. Representative example of a heatmap showing the clustering result obtained by consensus K-means clustering of TCGA-BLCA. The matrix was ordered by the consensus clustering class assignment. The colored legend on top of the heatmap shows the cluster number depicted in the legend

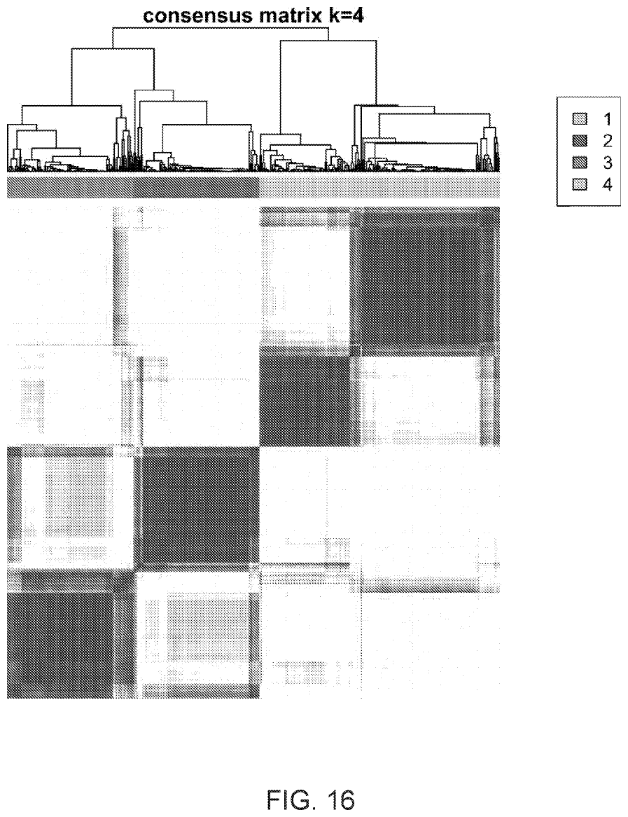

FIGS. 17A-17B. Representative example (A) Kaplan Meier curves of the overall survival outcome of TCGA-BLCA patients divided into groups of different AHR activation profiles by consensus K-means clustering. The p-values represent the probability of the age-adjusted cox proportional hazard. B) Box plot representation of the AHR activation score in TCGA BLCA subtypes determined by consensus K-means clustering. Group comparisons were performed by a Wilcox-sum rank test.

FIG. 18. Circular bar-plot representation of the Biological Process Activity score (BPA) for the different gene ontology groups representing AHR biological functions in the TCGA-BLCA determined by consensus K-means clustering. Each bar represents an ontology term. The height of the bar represents the value of the score. The black ring represents zero and all bars facing inward represent BPAs of negative values and bars facing outwards represent the BPAs with positive values. The colors of each bar correspond to the AHR biological process it belongs to.

FIG. 19. Heatmap representation of the log 2 counts per million normalized counts of the AHR biomarkers overlapping between the lasso and RFE feature selection methods, comprising the AHR signature for TCGA-BLCA determined by consensus K-means clustering. The colored bar on top represents the class assignment of the different tumor samples.

FIG. 20. Representative example of a heatmap showing the clustering result obtained by consensus NMF clustering of TCGA-BLCA. The matrix was ordered by the consensus clustering class assignment.

FIGS. 21A-21B. Representative example (A) Kaplan Meier curves of the overall survival outcome of TCGA-BLCA patients divided into groups of different AHR activation profiles by consensus NMF clustering. The p-values represent the probability of the age-adjusted cox proportional hazard. (B) Box plot representation of the AHR activation score in TCGA BLCA subtypes determined by consensus NMF clustering. The horizontal line represents the average AHR score across all tumor samples. The p-values represent the comparison of each group to mean of AHR expression in all tumor samples performed by a Wilcox-sum rank test.

FIG. 22. Circular bar-plot representation of the Biological Process Activity score (BPA) for the different gene ontology groups representing AHR biological functions in the TCGA-BLCA determined by consensus NMF clustering. Each bar represents an ontology term. The height of the bar represents the value of the score. The black ring represents zero and all bars facing inward represent BPAs of negative values and bars facing outwards represent the BPAs with positive values. The colors of each bar correspond to the AHR biological process it belongs to.

FIG. 23. Heatmap representation of the log 2 counts per million normalized counts of the AHR biomarkers overlapping between the lasso and RFE feature selection methods, comprising the AHR signature for TCGA-BLCA determined by consensus NMF clustering. The colored bar on top represents the class assignment of the different tumor samples.

FIG. 24. Heatmap representation of the standardized RPPA features that differentiate between the AHR subgroups of TCGA BLCA determined by consensus clustering. The first top colored bar above the heatmap represents the class assignment of the NMF consensus clustering and the second top colored bar represents the class assignments of K-means consensus clustering for the different tumor samples.

FIG. 25. Forest plot representation showing the distribution of AHR active groups in non-small cell lung cancer of both TCGA-LLUAD (adenocarcinoma) and TCGA-LUSC (squamous cell carcinoma). The AHR high and low groups were determined based on a cutoff value of 0.1 based on 1000 simulations of the null distribution, where no change in gene expression is present.

FIG. 26. Kaplan Meier curves of the overall survival outcome of the AHR high and low groups in TCGA-LUAD and TCGA-LUSC. The p-values represent the probability of the age-adjusted cox proportional hazard.

FIGS. 27A-27C. Boxplot representations showing the mutational distribution of, A) ALK, B) EGFR and C) ROS1 in the AHR high and low groups of both TCGA-LUAD and TCGA-LUSC. The colored dots represent the type of the mutation that a single patient harbors in the respective tumor/AHR group.

FIGS. 28A-28B. Box plot representation of the log 2 counts per million normalized counts of PD-L1 in the AHR high and low groups of, (A) TCGA-LUAD and (B) TCGA-LUSC. Group comparisons were performed by a Wilcox-sum rank test.

FIG. 29A-29B. Box plot representation of the AHR activation score of the clinically defined TCGA-HNSC cancer that are positive or negative for HPV based on an in situ hybridization test (A) or a more specific p16 assay (B). The HPV positive or negative groups were divided into AHR high and low groups based on a cutoff value of 0.1 based on 1000 simulations of the null distribution, where no change in gene expression is present.

FIG. 30. Kaplan Meier curves of the overall survival outcome of the AHR high and low groups in the HPV clinical subtypes of TCGA-HNSC. The p-values represent the probability of the age-adjusted cox proportional hazard.

FIG. 31. Shows barcode plots showing the direction of regulation of the AHR biomarkers after performing differential gene regulation of: A) HepG2 cells exposed to 2 uM of BaP for 24 hours (GSE28878), B) Human skin fibroblast cells derived from hypospadias patients exposed to 0.01 nM 170-estradiol (E2) 24 hours (GSE35034), and C) AHR activation after nivolumab treatment in advanced melanoma patients (GSE91061). The x-axis represents the moderated t-statistic values for all genes in the comparison. The darker grey scales represent the lower and upper quartiles of all the genes. The vertical barcode lines represent the distribution of the AHR signature genes. The worm line representation above the barcode shows the direction of regulation of the AHR signature.

FIG. 32. Block diagram of the system in accordance with the aspects of the disclosure. CPU: Central Processing Unit (“processor”)

FIG. 33. Flow chart of an embodiment for determining AHR activation signature.

FIG. 34. Flow chart of an embodiment for determining AHR activation status of a sample.

### DETAILED DESCRIPTION

### Definitions

As used herein, the term “about” refers to a variation within approximately +10% from a given value.

An “AHR signaling modulator” or an “AHR modulator” as used herein, refers to a modulator which affects AHR signaling in a cell. In some embodiments, an AHR signaling modulator exhibits direct effects on AHR signaling. In some embodiments, the direct effect on AHR is mediated through direct binding to AHR. In some embodiments, a direct modulator exhibits full or partial agonistic and/or antagonistic effects on AHR. In some embodiments, an AHR modulator is an indirect modulator.

In some embodiments, an AHR signaling modulator is a small molecule compound. The term “small molecule compound” herein refers to small organic chemical compound, generally having a molecular weight of less than 2000 daltons, 1500 daltons, 1000 daltons, 800 daltons, or 600 daltons.

In some embodiments, an AHR modulator comprises a 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, a 2-heteroaryl-3-oxo-2,3-dihydro-4-carboxamide compound, PDM 2, 1,3-dichloro-5-[(1E)-2-(4-methoxyphenyl)ethenyl]-benzene, a-Naphthoflavone, 6, 2′,4′-Trimethoxyflavone, CH223191, a tetrahydropyridopyrimidine derivative, StemRegenin-1, CH223191, GNF351, CB7993113 HP163. PX-A590, PX-A548. PX-A275, PX-A758, PX-A446, PX-A24590, PX-A25548, PX-A25275, PX-A25758, PX-A26446, an Indole AHR inhibitor, and an oxazole-containing (OxC) compound.

In some embodiments, a direct AHR modulator comprises:

(a) Drugs: e.g. Omeprazole, Sulindac, Leflunomide, Tranilast, Laquinimod, Flutamide, Nimodipine, Mexiletine, 4-Hydroxy-Tamoxifen, Vemurafenib etc.

(b) Synthethic compounds: e.g. 10-Chloro-7H-benzimidazo[2,1-a]benz[de]isoquinolin-7-one (10-CI-BBQ), Pifithrin-α hydrobromide,

(c) Natural compounds: e.g., kynurenine, kynurenic acid, cinnabarinic acid, ITE, FICZ, indoles including indole-3-carbinol, indole-3-pyruvate, indole-aldehyde, microbial metabolites, dietary components, quercetin, resveratrol, curcurmin, or

(d) Toxic compounds: e.g. TCDD, cigarette smoke, 3-methylcholantrene, benzo(a)pyrene, 2,3,7,8-tetrachlorodibenzofuran, fuel emissions, halogenated and nonhalogenated aromatic hydrocarbon, pesticides.

In some embodiments, indirect AHR modulators affect AHR activation through modulation of the levels of AHR agonists or antagonists.

In some embodiments, the modulation of the levels of AHR agonists or antagonists is mediated through one or more of the following:

(a) regulation of enzymes modifying AHR ligands e.g. the cytochrome p450 enzymes by e.g. cytochrome p450 enzyme inhibitors including 3′methoxy-4′nitroflavone (MNF), alpha-naphthoflavone (a-NF), fluoranthene (FL), phenanthrene (Phe), pyrene (PY) etc.
(b) regulation of enzymes producing AHR ligands including direct and indirect inhibitors/activators/inducers of tryptophan-catabolizing enzymes e.g. IDO1 pathway modulators (indoximod, NLG802), IDO1 inhibitors (1-methyl-L-tryptophan, Epacadostat, PX-D26116, navoximod, PF-06840003, NLG-919A, BMS-986205, INCB024360A, KHK2455, LY3381916, MK-7162, TDO2 inhibitors (680C91, LM10, 4-(4-fluoropyrazol-1-yl)-1,2-oxazol-5-amine, fused imidazo-indoles, indazoles), dual IDO/TDO inhibitors (HTI-1090/SHR9146, DN1406131, RG70099, EPL-1410), immunotherapy incuding immune checkpoint inhibition, vaccination, and cellular therapies, chemotherapy, immune stimulants, radiotherapy, exposure to UV light, and targeted therapies such as e.g. imatinib etc.

In some embodiments, indirect AHR modulators affect AHR activation through modulation of the expression of the AHR including e.g. HSP 90 inhibitors such as 17-allylamino-demethoxygeldanamycin (17-AAG), celastrol.

In some embodiments, indirect AHR modulators affect AHR activation by affecting binding partners/co-factors modulating the effects of AHR including e.g. estrogen receptor alpha (ESRI).

Examples of AHR modulators are listed in U.S. Pat. No. 9,175,266, US2019/225683, WO2019101647AL, WO2019101642A1, WO2019101643A1, WO2019101641AL, WO2018146010A1, AU2019280023A1, WO2020039093A1, WO2020021024A1, WO2019206800A1, WO2019185870A1, WO2019115586A1, EP3535259A1, WO2020043880A1 and EP3464248A1, all of which are incorporated by reference in their entirety.

As used herein, the phrase “biological sample” refers to any sample taken from a living organism. In some embodiments, the living organism is a human. In some embodiments, the living organism is a non-human animal.

In some embodiments, a biological sample includes, but is not limited to, biological fluids comprising biomarkers, cells, tissues, and cell lines. In some embodiments, a biological sample includes, but is not limited to, primary cells, induced pluripotent cells (IPCs), hybridomas, recombinant cells, whole blood, stem cells, cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cell types thereof.

The phrase “computer readable medium” refers to a computer readable storage device or a computer readable signal medium. A computer readable storage device, may be, for example, a magnetic, optical, electronic, electromagnetic, infrared, or semiconductor system, apparatus, or device, or any suitable combination of the foregoing: however, the computer readable storage device is not limited to these examples except a computer readable storage device excludes computer readable signal medium. Additional examples of the computer readable storage device can include: a portable computer diskette, a hard disk, a magnetic storage device, a portable compact disc read-only memory (CD-ROM), a random access memory (RAM), a read-only memory (ROM), an erasable programmable read-only memory (EPROM or Flash memory), an optical storage device, or any appropriate combination of the foregoing; however, the computer readable storage device is also not limited to these examples. Any tangible medium that can contain, or store, a program for use by or in connection with an instruction execution system, apparatus, or device could be a computer readable storage device.

A computer readable signal medium may include a propagated data signal with computer readable program code embodied therein, such as, but not limited to, in baseband or as part of a carrier wave. A propagated signal may take any of a plurality of forms, including, but not limited to, electro-magnetic, optical, or any suitable combination thereof. A computer readable signal medium may be any computer readable medium (exclusive of computer readable storage device) that can communicate, propagate, or transport a program for use by or in connection with a system, apparatus, or device. Program code embodied on a computer readable signal medium may be transmitted using any appropriate medium, including but not limited to wireless, wired, optical fiber cable, RF. etc., or any suitable combination of the foregoing.

In some embodiments, the term “condition” includes, but is not limited to a disease, or a cellular state. In some embodiments, the condition comprises cancer, diabetes, autoimmune disorder, degenerative disorder, inflammation, infection, drug treatment, chemical exposure, biological stress, mechanical stress, or environmental stress.

In some embodiments, the condition is cancer. In some embodiments, the cancer is selected from Adrenocortical carcinoma(ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO). Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM).

In some embodiments, different outcomes of a condition comprise positive response to treatment and no response to treatment. In some embodiments, different outcomes of a condition comprise favorable prognosis and unfavorable prognosis. In some embodiments, the different outcomes of the condition comprise death from the condition and survival from the condition. In some embodiments, the different outcomes of the condition are not binary, i.e., there are different levels, degrees or gradations between two opposite outcomes.

The phrase “fold change” refers to the ratio between the value of a specific biomarker in two different conditions. In some embodiments, one of the two conditions could be a control. The phrase “absolute fold change” is used herein in the case of comparing the log transformed value of a specific biomarker between two conditions. Absolute fold change is calculated by raising the exponent of the logarithm to the fold change value and then reporting the modulus of the number.

As used herein, the phrase “functional outcome” or “functional group” refers to groups of biomarkers represented by common gene ontology (GO) terms. In some embodiments, the gene ontology terms include terms that describe biological processes. In some embodiments, the gene ontology terms include terms that describe molecular functions. In some embodiments, the gene ontology terms include terms that describe cellular components. In some embodiments, the phrase “functional outcome” or “functional group” includes, but is not limited to, angiogenesis, positive regulation of vasculature development, reactive oxygen species metabolic process, reactive nitrogen species metabolic process, organic hydroxy compound metabolic process, xenobiotic metabolic process, cellular ketone metabolic process, toxin metabolic process, alcohol metabolic process, response to drug, response to toxic substance, response to oxidative stress, response to xenobiotic stimulus, response to acid chemical, response to extracellular stimulus, cellular response to biotic stimulus, cellular response to external stimulus, positive regulation of response to external stimulus, response to immobilization stress, response to hyperoxia, cellular response to extracellular stimulus, regulation of hemopoiesis, regulation of blood coagulation, regulation of hemostasis, regulation of coagulation, regulation of homeostatic process, response to temperature stimulus, regulation of blood pressure, blood coagulation, positive regulation of cytokine production, cytokine biosynthetic process, positive regulation of defense response, chemokine production, regulation of response to cytokine stimulus, regulation of chemotaxis, lipid localization, lipid storage, positive regulation of lipid localization, regulation of lipid localization, negative regulation of transport, positive regulation of cell-cell adhesion, myeloid leukocyte migration, positive regulation of locomotion, positive regulation of cellular component movement, regulation of hormone levels, hormone-mediated signaling pathway, positive regulation of smooth muscle cell proliferation, smooth muscle cell proliferation, positive regulation of cell cycle, response to oxygen levels, regulation of DNA binding transcription factor activity, response to transforming growth factor beta, negative regulation of response to external stimulus, ovulation cycle, response to radiation, and sex differentiation.

The term “memory” as used herein comprises program memory and working memory. The program memory may have one or more programs or software modules. The working memory stores data or information used by the CPU in executing the functionality described herein.

The term “processor” may include a single core processor, a multi-core processor, multiple processors located in a single device, or multiple processors in wired or wireless communication with each other and distributed over a network of devices, the Internet, or the cloud. Accordingly, as used herein, functions, features or instructions performed or configured to be performed by a “processor”, may include the performance of the functions, features or instructions by a single core processor, may include performance of the functions, features or instructions collectively or collaboratively by multiple cores of a multi-core processor, or may include performance of the functions, features or instructions collectively or collaboratively by multiple processors, where each processor or core is not required to perform every function, feature or instruction individually. The processor may be a CPU (central processing unit). The processor may comprise other types of processors such as a GPU (graphical processing unit). In other aspects of the disclosure, instead of or in addition to a CPU executing instructions that are programmed in the program memory, the processor may be an ASIC (application-specific integrated circuit), analog circuit or other functional logic, such as a FPGA (field-programmable gate array), PAL (Phase Alternating Line) or PLA (programmable logic array).

The CPU is configured to execute programs (also described herein as modules or instructions) stored in a program memory to perform the functionality described herein. The memory may be, but not limited to, RAM (random access memory), ROM (read-only memory) and persistent storage. The memory is any piece of hardware that is capable of storing information, such as, for example without limitation, data, programs, instructions, program code, and/or other suitable information, either on a temporary basis and/or a permanent basis.

The term “treatment,” as used herein, refers to a reduction, attenuation, diminuation and/or amelioration of the symptoms of a disease. In some embodiments, an effective treatment for cancer achieves, for example, a shrinking of the mass of a tumor and the number of cancer cells. In some embodiments, a treatment avoids (prevents) and reduces the spread of a disease. In some embodiments, the disease is cancer, and treatment affects cancer metastases and/or the formation thereof. In some embodiments, a treatment is a naive treatment (before any other treatment of a disease had started), or a treatment after the first round of treatment (e.g. after surgery or after a relapse). In some embodiments, a treatment is a combined treatment, involving, for example, chemotherapy, surgery, and/or radiation treatment. In some embodiments, treatment can also modulate auto-immune response, infection and inflammation.

### General Description

Aryl hydrocarbon receptor (AHR) target gene expression is context-specific, and therefore an AHR activation signature consisting of diverse AHR target genes is required to efficiently detect AHR activation across different cells/tissues and in response to diverse AHR ligands. It is therefore an object of the present disclosure, to provide transcriptional AHR activation signatures that enable reliable detection of AHR activation in various human tissues and under different conditions, while maintaining sufficient complexity. Furthermore, additional genes are sought after as markers that help to further understand the complex functions of AHR in particular the context of diseases and conditions related with AHR.

The present disclosure relates to the generation and uses of an improved set (or “panel”) of biomarkers (also “markers” or “genes”) that are AHR target genes, designated as “AHR biomarkers.” The AHR biomarkers described herein allow one to efficiently determine AHR activation groups and sub-groups, in particular for an improved classification of tumors. As used herein, AHR activation groups are called “AHR activation signatures.” The AHR biomarkers comprise markers that are important in diagnosis and therapy, for example for selecting patients for treatment with AHR activation modulating interventions, and monitoring of therapy response. In some embodiments, the AHR biomarkers are selected from biomarkers listed in Table 1.

TABLE 1
AHR biomarkers are indicated with their HUGO Gene Nomenclature Committee
(HGNC)-approved name and Entrez database ID for human copies.

| | Entrez ID |
|---|---|
| Gene | (homo sapiens) |
| actin alpha 2, smooth muscle (ACTA2) | 59 |
| adhesion molecule with Ig like domain 2 (AMIGO2) | 347902 |
| adrenomedullin (ADM) | 133 |
| aldehyde dehydrogenase 3 family member A1 (ALDH3A1) | 218 |
| amphiregulin (AREG) | 374 |
| aquaporin 3 (Gill blood group) (AQP3) | 360 |
| arginase 2 (ARG2) | 384 |
| aryl hydrocarbon receptor (AHR) | 196 |
| aryl-hydrocarbon receptor repressor (AHRR) | 57491 |
| ATP binding cassette subfamily C member 4 (ABCC4) | 10257 |
| ATP binding cassette subfamily G member 2 (Junior blood group) | 9429 |
| (ABCG2) | |
| ATP synthase inhibitory factor subunit 1 (ATP5IF1) | 93974 |
| ATP synthase membrane subunit e (ATP5ME) | 521 |
| ATPase H+ transporting accessory protein 2 (ATP6AP2) | 10159 |
| ATPase H+/K+ transporting non-gastric alpha2 subunit (ATP12A) | 479 |
| B cell linker (BLNK) | 29760 |
| BAF chromatin remodeling complex subunit BCL11B (BCL11B) | 64919 |
| BCL2 apoptosis regulator (BCL2) | 596 |
| BCL6 transcription repressor (BCL6) | 604 |
| BRCA1 DNA repair associated (BRCA1) | 672 |
| C-C motif chemokine ligand 5 (CCL5) | 6352 |
| C-X-C motif chemokine ligand 2 (CXCL2) | 2920 |
| caspase recruitment domain family member 11 (CARD11) | 84433 |
| caveolin 1 (CAV1) | 857 |
| CD3e molecule (CD3E) | 916 |
| CD36 molecule (CD36) | 948 |
| CD8a molecule (CD8A) | 925 |
| coagulation factor III, tissue factor (F3) | 2152 |
| corticotropin releasing hormone (CRH) | 1392 |
| cyclin D1 (CCND1) | 595 |
| cyclin dependent kinase 4 (CDK4) | 1019 |
| cyclin dependent kinase inhibitor 1A (CDKN1A) | 1026 |
| cystic fibrosis transmembrane conductance regulator (CFTR) | 1080 |
| cytochrome b-245 beta chain (CYBB) | 1536 |
| cytochrome P450 family 1 subfamily A member 1 (CYP1A1) | 1543 |
| cytochrome P450 family 1 subfamily A member 2 (CYP1A2) | 1544 |
| cytochrome P450 family 1 subfamily B member 1 (CYP1B1) | 1545 |
| cytochrome P450 family 19 subfamily A member 1 (CYP19A1) | 1588 |
| cytochrome P450 family 2 subfamily B member 6 (CYP2B6) | 1555 |
| cytochrome P450 family 2 subfamily E member 1 (CYP2E1) | 1571 |
| cytochrome P450 family 3 subfamily A member 4 (CYP3A4) | 1576 |
| dickkopf WNT signaling pathway inhibitor 3 (DKK3) | 27122 |
| distal-less homeobox 3 (DLX3) | 1747 |
| DNA polymerase kappa (POLK) | 51426 |
| dual oxidase 2 (DUOX2) | 50506 |
| early growth response 1 (EGR1) | 1958 |
| EBF transcription factor 1 (EBF1) | 1879 |
| endothelin 1 (EDN1) | 1906 |
| epidermal growth factor receptor (EGFR) | 1956 |
| epiregulin (EREG) | 2069 |
| epithelial mitogen (EPGN) | 255324 |
| estrogen receptor 1 (ESR1) | 2099 |
| F-box protein 32 (FBXO32) | 114907 |
| Fas cell surface death receptor (FAS) | 355 |
| FAT atypical cadherin 1 (FAT1) | 2195 |
| fibroblast growth factor receptor 2 (FGFR2) | 2263 |
| FIG4 phosphoinositide 5-phosphatase (FIG4) | 9896 |
| filaggrin (FLG) | 2312 |
| forkhead box A1 (FOXA1) | 3169 |
| forkhead box Q1 (FOXQ1) | 94234 |
| formyl peptide receptor 2 (FPR2) | 2358 |
| Fos proto-oncogene, AP-1 transcription factor subunit (FOS) | 2353 |
| G protein subunit alpha 13 (GNA13) | 10672 |
| GATA binding protein 3 (GATA3) | 2625 |
| glutamine amidotransferase like class 1 domain containing 3A | 8209 |
| (GATD3A) | |
| glutathione S-transferase alpha 2 (GSTA2) | 2939 |
| glutathione S-transferase mu 1 (GSTM1) | 2944 |
| growth factor independent 1 transcriptional repressor (GFI1) | 2672 |
| growth hormone receptor (GHR) | 2690 |
| heat shock protein family B (small) member 2 (HSPB2) | 3316 |
| heme oxygenase 1 (HMOX1) | 3162 |
| hes family bHLH transcription factor 1(HES1) | 3280 |
| hydroxysteroid 17-beta dehydrogenase 4 (HSD17B4) | 3295 |
| hypoxia inducible factor 1 subunit alpha (H1F1A) | 3091 |
| IKAROS family zinc finger 3 (IKZF3) | 22806 |
| inhibitor of DNA binding 1, HLH protein (ID1) | 3397 |
| inhibitor of DNA binding 2 (ID2) | 3398 |
| insulin induced gene 1 (INSIG1) | 3638 |
| insulin like growth factor 2 (IGF2) | 3481 |
| insulin like growth factor binding protein 1(IGFBP1) | 3484 |
| interferon gamma (IFNG) | 3458 |
| interferon regulatory factor 8 (IRF8) | 3394 |
| interleukin 1 beta (IL1B) | 3553 |
| interleukin 1 receptor type 2 (IL1R2) | 7850 |
| interleukin 2 (IL2) | 3558 |
| interleukin 6 (IL6) | 3569 |
| jagged canonical Notch ligand 1 (JAG1) | 182 |
| junction plakoglobin (JUP) | 3728 |
| KIAA1549 (KIAA1549) | 57670 |
| KIT proto-oncogene, receptor tyrosine kinase (KIT) | 3815 |
| kynurenine 3-monooxygenase (KMO) | 8564 |
| latent transforming growth factor beta binding protein 1 (LTBP1) | 4052 |
| leptin receptor (LEPR) | 3953 |
| LIF receptor alpha (L1FR) | 3977 |
| lipoprotein lipase (LPL) | 4023 |
| luteinizing hormone/choriogonadotropin receptor (LHCGR) | 3973 |
| LYN proto-oncogene, Src family tyrosine kinase (LYN) | 4067 |
| lysine demethylase 1A (KDM1A) | 23028 |
| major histocompatibility complex, class II, DR beta 4 (HLA-DRB4) | 3126 |
| matrix metallopeptidase 1 (MMP1) | 4312 |
| midline 1 (MID1) | 4281 |
| musashi RNA binding protein 2 (MSI2) | 124540 |
| MYC proto-oncogene, bHLH transcription factor (MYC) | 4609 |
| N-myc downstream regulated I (NDRG1) | 10397 |
| NAD(P) dependent steroid dehydrogenase-like (NSDHL) | 50814 |
| NAD(P)H quinone dehydrogenase 1 (NQO1) | 1728 |
| Nanog homeobox (NANOG) | 79923 |
| neural precursor cell expressed, developmentally down-regulated 9 | 4739 |
| (NEDD9) | |
| neuronal pentraxin 1 (NPTX1) | 4884 |
| nitric oxide synthase 1 (NOS1) | 4842 |
| nitric oxide synthase 3 (NOS3) | 4846 |
| nuclear factor, erythroid 2 like 2 (NFE2L2) | 4780 |
| nuclear receptor coactivator 2 (NCOA2) | 10499 |
| nuclear receptor corepressor 2 (NCOR2) | 9612 |
| nuclear receptor interacting protein 1 (NR1P1) | 8204 |
| nuclear receptor subfamily 1 group H member 3 (NR1H3) | 10062 |
| nuclear receptor subfamily 1 group H member 4 (NR1H4) | 9971 |
| nuclear receptor subfamily 3 group C member 1 (NR3C1) | 2908 |
| ovo like transcriptional repressor 1 (OVOL1) | 5017 |
| paired box 5 (PAX5) | 5079 |
| patatin like phospholipase domain containing 7 (PNPLA7) | 375775 |
| PDS5 cohesin associated factor B (PDS5B) | 23047 |
| period circadian regulator 1 (PER1) | 5187 |
| phosphodiesterase 2A (PDE2A) | 5138 |
| phosphoenolpyruvate carboxykinase 1 (PCK1) | 5105 |
| phosphoenolpyruvate carboxykinase 2, mitochondrial (PCK2) | 5106 |
| phosphoglycerate dehydrogenase (PHGDH) | 26227 |
| phospholipase A2 group IVA (PLA2G4A) | 5321 |
| piwi like RNA-mediated gene silencing 1 (PIWIL1) | 9271 |
| piwi like RNA-mediated gene silencing 2 (PIWIL2) | 55124 |
| PPARG coactivator 1 alpha (PPARGC1A) | 10891 |
| PR/SET domain 1 (PRDM1) | 639 |
| prostaglandin-endoperoxide synthase 2 (PTGS2) | 5743 |
| R-spondin 3 (RSPO3) | 84870 |
| REL proto-oncogene, NF-kB subunit (REL) | 5966 |
| replication factor C subunit 3 (RFC3) | 5983 |
| retinoic acid receptor alpha (RARA) | 5914 |
| scavenger receptor class B member 1 (SCARB1) | 949 |
| scinderin (SCIN) | 85477 |
| serpin family B member 2 (SERPINB2) | 5055 |
| serpin family E member 1 (SERPINE1) | 5054 |
| sestrin 2 (SESN2) | 83667 |
| SH3 domain containing kinase binding protein 1 (SH3KBP1) | 30011 |
| SMAD family member 3 (SMAD3) | 4088 |
| SMAD family member 7 (SMAD7) | 4092 |
| small proline rich protein 2D (SPRR2D) | 6703 |
| solute carrier family 10 member 1 (SLC10A1) | 6554 |
| solute carrier family 3 member 2 (SLC3A2) | 6520 |
| solute carrier family 7 member 5 (SLC7A5) | 8140 |
| sortilin related receptor 1 (SORL1) | 6653 |
| SOS Ras/Rac guanine nucleotide exchange factor 1 (SOS1) | 6654 |
| stanniocalcin 2 (STC2) | 8614 |
| suppressor of cytokine signaling 2 (SOCS2) | 8835 |
| TCDD inducible poly(ADP-ribose) polymerase (TIPARP) | 25976 |
| thioredoxin reductase 1 (TXNRD1) | 7296 |
| thrombospondin 1 (THBS1) | 7057 |
| tight junction protein 1 (TJP1) | 7082 |
| TNF superfamily member 9 (TNFSF9) | 8744 |
| transforming growth factor beta induced (TGFBI) | 7045 |
| transglutaminase 1 (TGM1) | 7051 |
| trefoil factor 1 (TFF1) | 7031 |
| tyrosine hydroxylase (TH) | 7054 |
| UDP glucuronosyltransferase family 1 member A6 (UGT1A6) | 54578 |
| vav guanine nucleotide exchange factor 3 (VAV3) | 10451 |
| xanthine dehydrogenase (XDH) | 7498 |
| Zic family member 3 (ZIC3) | 7547 |

### A. Methods for Determining an AHR Activation Signature for a Condition

An aspect of the present disclosure is directed to methods for determining an AHR signature for a given condition. In some embodiments, the AHR signature for a condition is a subset of biomarkers listed in Table 1.

In some embodiments, the method for determining AHR activation signature for a condition comprises: (a) providing at least two biological samples of the condition, wherein the at least two biological samples represent at least two different outcomes for the condition; (b) detecting a biological state of each of the AHR biomarkers of Table 1 for the at least two biological samples; (c) categorizing the AHR biomarkers into at least two groups based on the change of biological state of each marker compared to a control: (d) categorizing the at least two groups into at least two subgroups based on at least one functional outcome of AHR signaling; and (e) designating the markers in the at least two subgroups that correlate with the at least two different outcomes as the AHR activation signature for the condition.

In some embodiments, the biological state detected at step (b) is RNA expression. In some embodiments, the detecting a biological state comprises measuring levels of the biological state. In some embodiments, RNA expression of a biomarker is detected by methods known in the art including, but not limited to, qPCR, RT-qPCR, RNA-Seq, and in-situ hybridization. In some embodiments, the biological state of all AHR biomarkers listed in Table 1 are detected or measured.

In some embodiments, the categorizing in step (c) is achieved by supervised clustering. In some embodiments, the categorizing in step (c) is achieved by unsupervised clustering. In some embodiments, the clustering method comprises one or more methods including, but not limited to, K-means clustering, hierarchical clustering, principle component analysis and non-negative matrix factorization. In some embodiments, the categorizing in step (c) is achieved by a machine learning algorithm.

In some embodiments, the categorizing in step (c) comprises grouping together AHR biomarkers that display at least 1.5 absolute fold upregulation in the biological state. In some embodiments, the categorizing in step (c) comprises grouping together AHR biomarkers that display at least 2 absolute fold, at least 2.5 absolute fold, at least 3 absolute fold, at least 3.5 absolute fold, at least 4 absolute fold, at least 4.5 absolute fold, or at least 5 absolute fold upregulation in the biological state.

In some embodiments, the categorizing in step (c) comprises grouping together AHR biomarkers that display at least 0.67 absolute fold down-regulation in the biological state. In some embodiments, the categorizing in step (c) comprises grouping together AHR biomarkers that display at least 1 absolute fold, 2 absolute fold, at least 2.5 absolute fold, at least 3 absolute fold, at least 3.5 absolute fold, at least 4 absolute fold, at least 4.5 absolute fold, or at least 5 absolute fold down-regulation in the biological state.

In some embodiments, the categorizing in step (d) is achieved by supervised clustering. In some embodiments, the categorizing in step (d) is achieved by unsupervised clustering. In some embodiments, the clustering method comprises one or more methods including, but not limited to, K-means clustering, hierarchical clustering, principle component analysis and non-negative matrix factorization. In some embodiments, the categorizing in step (d) is achieved by a machine learning algorithm.

In some embodiments, the methods of the present disclosure are used to sub-classify tumors/cancer patients based on molecular characteristics known to affect prognosis and therapy response. To obtain even higher granularity it is important to analyze AHR activity in tumor subgroups with specific clinical characteristics. In some embodiments, the AHR signature and the methods described herein are used to analyze and compare clinically defined subgroups of cancer entities, and correlate AHR activity with clinical outcome.

In some embodiments, the AHR activation signature comprises about 5, about 10, about 20, about 30 of the AHR biomarkers according to Table 1 or at least 10%. at least 20%, at least 30%, at least 40%, at least 50%, at least 60%, at least 70%, at least 80% or at least 90% or more or all of the AHR biomarkers according to Table 1.

In some embodiments, the AHR activation signature comprises an AHR signature listed in Table 2.

In some embodiments, the methods of the present disclosure are directed to determine a subset of AHR activation signature, called “an AHR subsignature,” wherein the AHR subsignature is enough to categorize a sample to a specific AHR subgroup within the AHR activation state.

In some embodiments, the AHR subsignature comprises at least one biomarker from the AHR activation signature. In some embodiment, the AHR subsignature comprises biomarkers that are about 10%, about 20%, about 50%, about 60%. about 70%, about 80%, about 90% or all biomarkers from the AHR activation signature. In some embodiments, the AHR subsignature is selected from Table 3. In some embodiments, the AHR subsignature is selected from Table 4.

### B. Alternative AHR Signatures

Another aspect of the disclosure is directed to determining an alternative AHR activation signature based on a second biological state that is different than the first biological state used in determining the AHR activation signature. In some embodiments, an AHR activation signature (a first or primary AHR activation signature) is determined for a condition based on a biological state (e.g., RNA expression) and functional outcome characterization of samples for the condition as described above in Section A. Further, the same samples used in generating the AHR activation signature based on the first biological state (e.g., RNA expression) are subjected to another 'omics analysis including, but not limited to genomics, epigenomics, lipidomics, proteomics, transcriptomics, metabolomics and glycomics analysis. Then, the results of the 'omics analysis is correlated with the groups determined by the first/primary AHR activation signature, thereby identifying an alternative (second/secondary) AHR activation signature. The alternative AHR signature is equivalent to the first AHR activation signature in that it allows determination of AHR activation state and characterization of a given sample (e.g., in terms of the outcome of the condition). In some embodiments, once a first AHR activation signature and a second AHR activation signature is determined/defined for a given condition, either AHR activation signature can be utilized to a) determine the AHR activation state, or b) category based on the functional and clinical outcome of the condition. In a specific embodiment, the first AHR activation signature is based on RNA expression, and the second AHR activation signature is based on protein analysis. Alternative AHR signatures are useful for use on samples where, e.g., RNA amount or quality is not good enough for RNA expression analyses (e.g., paraffin-embedded samples, frozen samples). Alternative AHR signatures may also lead to development of other diagnostic techniques (e.g., a protein-based assay looking at the alternative AHR signature of a condition based on proteomics).

In some embodiments, an alternative AHR signature is determined based on a second biological state which includes, but is not limited to, one of mutation state, methylation state, copy number, protein expression, metabolite abundance, and enzyme activity. In some embodiments, the second biological state of at least one biomarker is correlated with the least two subgroups that correlate with the at least two different outcomes. In some embodiments, the second biological state is determined for markers that are not limited to the biomarkers listed in Table 1.

In some embodiments, the alternative AHR signature comprises an alternative AHR signature listed in Table 5. In some embodiments, the alternative AHR signature comprises an alternative AHR signature listed in Table 6.

### C. Methods for Determining the AHR Activation State of a Biological Sample

Another aspect of the instant disclosure is directed to methods for determining the AHR activation state of a biological sample based on a given AHR activation signature specific for a condition. In some embodiments. the biological sample is taken from a subject. In some embodiments, a biological state is determined/measured for AHR biomarker of the given AHR activation signature.

In some embodiments, the AHR activation signature is a subset of AHR biomarkers listed in Table 1. In some embodiments, the AHR activation signature has been previously determined by one or more methods described in Section A. In some embodiments, the AHR activation signature comprises an AHR signature listed in Table 2.

In some embodiments, the AHR activation signature is an alternative/secondary AHR activation signature. In some embodiments, the alternative/secondary AHR activation signature has been determined by one or more methods described in Section B. In some embodiments, the alternative AHR signature comprises an alternative AHR signature listed in Table 5. In some embodiments, the alternative AHR signature comprises an alternative AHR signature listed in Table 6.

In some embodiments, the biological state of each AHR biomarker is used to perform clustering of the AHR biomarkers into subgroups defined by the AHR activation signature. as described in Section A. In some embodiments, the AHR activation signature comprises an AHR signature listed in Table 2.

In some embodiments, the method further comprises treating the subject with an AHR signaling modulator (also “AHR modulator”). In some embodiments, the AHR signaling modulator is administered every day, every other day, twice a week, once a week or once a month. In some embodiments, the AHR signaling modulator is administered together with other drugs as part of a combination therapy.

In some embodiments, an effective amount of a AHR signaling modulator is about 0.01 mg/kg to 100 mg/kg. In other embodiments, the effective amount of an AHR signaling modulator is about 0.01 mg/kg, 0.05 mg/kg, 0.1 mg/kg, 0.2 mg/kg. 0.5 mg/kg, 1 mg/kg, 5 mg/kg, 8 mg/kg, 10 mg/kg, 15 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of AHR signaling modulator.

Another aspect of the disclosure relates to a method of treating and/or preventing an AHR-related disease or condition in a cell in a patient in need of said treatment. comprising performing a method according to the present invention, and providing a suitable treatment to said patient, wherein said treatment is based, at least in part, on the results of the method according to the present invention, such as providing a compound as identified or monitoring a treatment comprising the method(s) as described herein.

Another aspect of the present disclosure relates to a diagnostic kit comprising materials for performing a method according to the present invention in one or separate containers. optionally together with auxiliary agents and/or instructions for performing said method.

### D. Methods of Screening for Compounds that Modulate AHR Activity

Another aspect of the instant disclosure is directed to screening for or identifying compounds which modulate AHR activity. Another aspect of the instant disclosure is directed to methods for determining the effects of a compound on AHR activation status of a cell.

In some embodiments, a cell is treated with a candidate compound, and in the cell. a biological state of each AHR biomarker of a given AHR activation signature is determined/measured.

In some embodiments, the AHR signature is specific for a condition.

In some embodiments, the AHR activation signature is a subset of AHR biomarkers listed in Table 1. In some embodiments, the AHR activation signature has been previously determined by one or more methods described in Section A. In some embodiments, the AHR activation signature comprises an AHR signature listed in Table 2.

In some embodiments, the AHR activation signature is an alternative/secondary AHR activation signature. In some embodiments, the alternative/secondary AHR activation signature has been determined by one or more methods described in Section B.

In some embodiments, the alternative AHR signature comprises an alternative AHR signature listed in Table 5. In some embodiments, the alternative AHR signature comprises an alternative AHR signature listed in Table 6.

In some embodiments, the biological state of each AHR biomarker in the biological sample is compared to the biological state of each AHR biomarker in a control sample.

In some embodiments, the biological state of each AHR biomarker is used to perform clustering of the AHR biomarkers into subgroups defined by the AHR activation signature, as described in Section A, and thereby determining the effect of the compound on AHR activation status of the cell, and/or categorizing the compound based on AHR activation status of the cell.

### E. Processor and Computer-Readable Storage Device

In some embodiments, the processor, the computer-readable storage device or the method of the present disclosure (“the technology described herein”) are applied to discover an aryl hydrocarbon receptor (AHR) biomarkers and an AHR activation signature selected from the pool of AHR biomarkers.

Various aspects of the present disclosure may be embodied as a program. software, or computer instructions embodied or stored in a computer or machine usable or readable medium, or a group of media which causes the computer or machine to perform the steps of the method when executed on the computer, processor, and/or machine. A program storage device readable by a machine, e.g., a computer readable medium, tangibly embodying a program of instructions executable by the machine to perform various functionalities and methods described in the present disclosure is also provided.

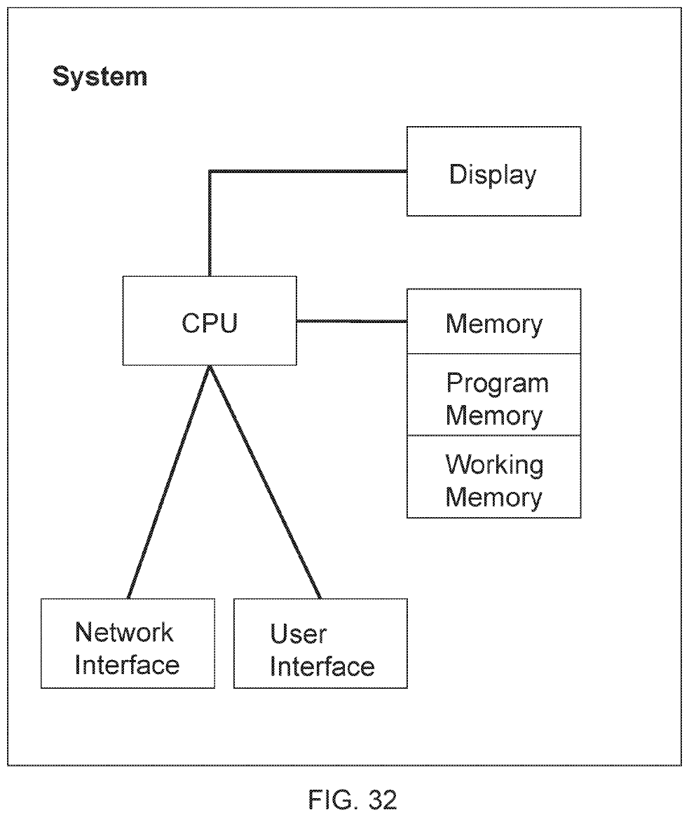

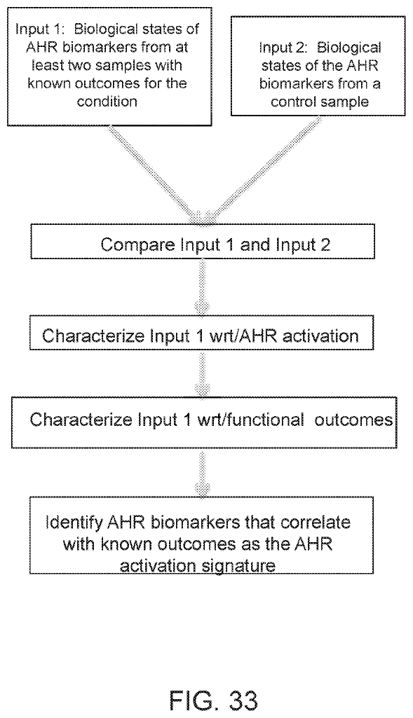

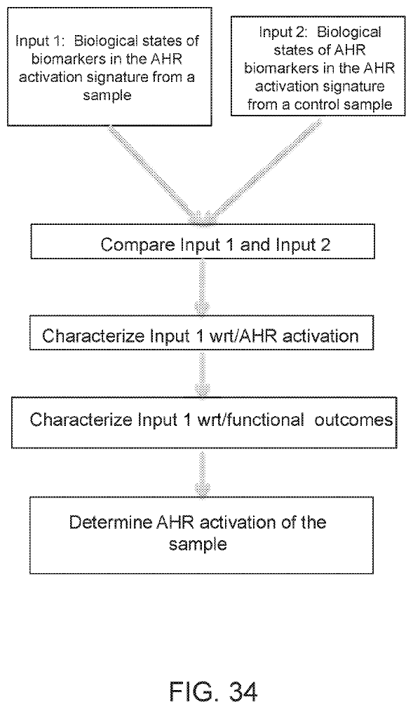

In some embodiments, the present disclosure includes a system comprising a CPU, a display, a network interface, a user interface, a memory, a program memory and a working memory (FIG. 32), where the system is programmed to execute a program, software, or computer instructions directed to methods or processes of the instant disclosure. Some embodiments are shown in FIG. 33 and FIG. 34.

In some embodiments, a processor is programmed to perform:

(i) comparing biological states of aryl hydrocarbon receptor (AHR) biomarkers from at least two samples with known outcomes with biological states of the AHR biomarkers from a control sample;

(ii) categorizing the at least two samples into at least two groups based on the comparison in step (i);

(iii) categorizing the result of step (ii) into at least two subgroups based on at least one functional outcome; and

(iv) identifying AHR biomarkers that correlate with the known outcomes.

In some embodiments, a computer-readable storage device comprises instructions to perform:

(i) comparing biological states of aryl hydrocarbon receptor (AHR) biomarkers from at least two samples with known outcomes with biological states of the AHR biomarkers from a control sample;

(ii) categorizing the at least two samples into at least two groups based on the comparison in step (i);

(iii) categorizing the result of step (ii) into at least two subgroups based on at least one functional outcome: and

(iv) identifying AHR biomarkers that correlate with the known outcomes.

In some embodiments, a processor is programmed to perform:

(i) comparing biological states of aryl hydrocarbon receptor (AHR) biomarkers of an AHR activation signature from a sample with biological states of the AHR biomarkers of the AHR activation signature from a control sample, wherein the AHR activation signature is specific for a condition;

(ii) categorizing the sample into a group based on the comparison in step (i);

(iii) categorizing the result of step (ii) into a subgroup based on at least one functional outcome; and

(iv) determining AHR activation state of the sample.

In some embodiments, a computer-readable storage device comprises instructions to perform:

(i) comparing biological states of aryl hydrocarbon receptor (AHR) biomarkers of an AHR activation signature from a sample with biological states of the AHR biomarkers of the AHR activation signature from a control sample, wherein the AHR activation signature is specific for a condition:

(ii) categorizing the sample into a group based on the comparison in step (i);

(iii) categorizing the result of step (ii) into a subgroup based on at least one functional outcome; and

(iv) determining AHR activation state of the sample.

### F. Additional Embodiments

In some embodiments, the disclosure is directed to a method for determining AHR activation signature for a biological sample, comprising detecting at least one biological state of at least one AHR biomarker according to Table 1 for said sample, identifying a change of said biological state of said at least one AHR biomarker compared to a house keeping gene or control biomarker, and assigning said at least one AHR biomarker to said AHR activation signature for said biological sample, if said at least one biomarker provides a significance of said AHR activation signature of p<0.05 at a minimal number of markers in the signature and/or a fold of change of said AHR activation signature of at least about 1.5 at a minimal number of markers in the signature in the case of up-regulation or of at least about 0.67 at a minimal number of markers in the signature in the case of down-regulation. The method can be in vivo or in vitro, including that the exposure of the cells/samples to AHR modulators could be from external sources, applied directly to the cells or as a result of an endogenous modulator that affects AHR activation both directly or indirectly.

In some embodiments, a housekeeping gene refers to a constitutive gene that is expressed in all cells of the biological sample to be analyzed. Usually housekeeping genes are selected by the person of skill based on their requirement for the maintenance of basic cellular function in the cells of the sample as analyzed under normal, and patho-physiological conditions (if present in the context of the analysis). Examples of housekeeping genes are known to the person of skill, and may involve the ones as disclosed, e.g. in Eisenberg E, Levanon E Y (October 2013). “Human housekeeping genes, revisited”. Trends in Genetics. 29 (10): 569-574.

An aspect of the method according to the present disclosure further involves a step of identifying at least one suitable housekeeping gene and/or at least one suitable control biomarker for the sample to be analyzed, comprising detecting the expression and/or biological function of a potentially suitable housekeeping gene and/or control biomarker in said sample, and identifying said housekeeping gene and/or control biomarker as suitable, if said expression and/or biological function does not change or substantially change over time, when compared to the markers of the respective AHR signature as analyzed (control biomarker). Another suitable marker is the non-mutated version of a marker of the respective AHR signature as analyzed. Therefore, control biomarkers can be markers independent from the AHR signature or be part of the signature itself (particularly in case of mutations).

In some embodiments, the biological state as detected is selected from mutations, nucleic acid methylation, copy numbers, expression, amount of protein, metabolite, and activity of said at least one AHR biomarker.

In some embodiments, the at least one AHR biomarker is then assigned to said AHR activation signature for said biological sample. For this, in one embodiment the marker must show an absolute fold of change of said AHR activation signature of at least about 1.5 at a minimal number of markers in the signature in the case of up-regulation or f at least about 0.67 at a minimal number of markers in the signature in the case of down-regulation. Thus, a panel is created that contains as few as possible markers (i.e. 1, 2, 3, etc.) based on the most “prominent” changes as identified. This embodiment is particularly useful in cases where only a few markers are selected, e.g. in the context of a kit of markers and/or a point of care test, without the necessity of substantial machinery and equipment. In some embodiments, the absolute fold of change of said AHR activation signature is at least about 1.5, at least about 1.8, at least about 2, and at least about 3 or more in the case of up-regulation, or wherein said absolute fold of change of said AHR activation signature is at least about 0.67, at least about 0.57, at least about 0.25 or more in the case of down regulation.

In some embodiments, the AHR activation signature provides a significance of p<0.05, p<0.01, p<0.001, or p<0.0001 or at least an absolute fold of change of said AHR activation signature of at least about 1.5 in case of up-regulation or at least an absolute fold change of at least about 0.67 in the case of down regulation at a minimal number of markers in the signature.

In some embodiments, the AHR activation signature comprises about 5, about 10, about 20, about 30 of said AHR biomarkers according to table 1 or at least 10%. at least 20%, at least 30%, at least 40%, at least 50%, at least 60%. at least 70%, at least 80% or at least 90% or more or all of said AHR biomarkers according to Table 1.

In some embodiments, the AHR activation signature is identified in a sample under physiological conditions or under disease conditions, for example, in biological safety screenings, toxicology studies, cancer, autoimmune disorders, degeneration, inflammation and infection, or under stress conditions, for example, biological, mechanical and environmental stresses.

In some embodiments, the method further comprises the step of using the AHR activation signature for unsupervised clustering or supervised classification of the samples into AHR activation subgroups.

In some embodiments, the method further comprises a step of using an AHR activation signature for unsupervised clustering or supervised classification of said samples into AHR activation subgroups. Respective methods are known to the person of skill for example K-means clustering, hierarchical clustering, principle component analysis and non-negative matrix factorization. Clustering of the biomarkers will depend on the sample and the circumstances to be analyzed, and may be based on the biological function of the biomarkers, and/or the respective functional subgroup of the AHR signature or other groups of interest, e.g., the signaling pathway or network. The AHR signature as established is also capable of detecting AHR activation across different cell/tissue types and in response to diverse ligands. Using the AHR signature, it is possible to determine AHR activation sub-groups by unsupervised clustering methods, which can be utilized for classification of samples. This is important for example, in terms of selecting patients for treatment with AHR activation modulating interventions, and monitoring of therapy response.

In some embodiments, the AHR activation signature or AHR activation subgroups are further used to define AHR activation modulated functions, for example, angiogenesis, drug metabolism, external stress response, hemopoiesis, lipid metabolism, cell motility, and immune modulation.

In another aspect, the disclosure is directed to a method for monitoring AHR activation in a biological sample in response to at least one compound, comprising performing the method for determining AHR activation signature on samples that have been obtained during the course of contacting said sample with at least one pharmaceutically active compound, toxin or other modulator compound, wherein said modulator is preferably selected from an inhibitor or an agonist of said biological state.

In some embodiments, the method for monitoring AHR activation in a biological sample in response to at least one modulator compound comprises performing the method according to the present invention on biological samples/samples that have been obtained during the course of contacting said sample with at least one modulator. The modulator compound can be directly applied to the sample in vitro or through different routes of administration, for example, parenteral preparations, ingestion, topical application, vaccines, i.v., or others, wherein a change in the AHR activation in the presence of said at least one compound compared to the absence of said at least compound indicates an effect of said at least one compound on said AHR activation. In some embodiments, this modulator can be used in additional steps of the method where a classifier is used, or activation is evaluated based on the signature compared to housekeeping genes or control biomarkers as disclosed herein.

In some embodiment, the uses of the AHR-signature also include a method for monitoring an AHR-related disease or condition or function or effect in a cell, comprising performing a method according to the present invention, providing at least one modulator compound to said cell and detecting the change in at least one biological state of the genes of the AHR-signature in said cell in response to said at least one compound, wherein a change in the at least one biological state of the genes of said signature in the presence of said at least one compound compared to the absence of said at least compound indicates an effect of said at least one compound on said AHR-related disease or condition or function or effect.

In some embodiments, the present disclosure relates to a method for screening for a modulator compound of AHR activation genes, comprising performing the method according to the present invention, and further comprising contacting at least one candidate modulator compound with said biological sample, wherein a change in the biological state of said at least one AHR biomarker of said signature in the presence of said at least one compound compared to the absence of said at least compound identifies a modulator. The modulator compound of AHR activation genes can modulate said genes directly or indirectly, i.e., by acting on AHR directly, or indirectly by acting on a signaling pathway upstream of the AHR marker.

In some embodiments, the present disclosure relates to an in-vitro method for screening for a modulator of the expression of AHR-regulated genes, comprising contacting a cell with at least one candidate modulator compound, and detecting at least one of mutations, nucleic acid methylation, copy numbers, expression, amount of protein, metabolites and activity of said genes of the AHR-signature according to table 1, wherein a change as detected of about 5, about 10, about 20, about 30 of said AHR biomarkers according to table 1 or at least 10%, at least 20%, at least 30%, at least 40%. at least 50%, at least 60%. at least 70%, at least 80% or at least 90% or more or all of said AHR biomarkers according to Table 1 in the presence of said at least one compound compared to the absence of said at least compound identifies a modulator. This modulator in preferred embodiments can be used in additional steps of the method where a classifier is used or activation is evaluated based on the signature compared to housekeeping genes or control biomarkers as disclosed herein.

In another aspect, the present disclosure relates to a method for testing the biological safety of a compound, comprising performing a method according to the present invention, and further comprising the step of concluding on the safety of said compound based on said effect as identified. Because of the known relation of AHR to toxic compounds, another advantageous use is a method for testing the biological safety of a compound, comprising performing a method according to the present invention, and further comprising the step of concluding on the safety of said compound based on said effect as identified.

Another aspect of the present invention then relates to a method for producing a pharmaceutical preparation, wherein said compound/modulator as identified (screened) is further formulated into a pharmaceutical preparation by admixing said (at least one) compound as identified (screened) with a pharmaceutically acceptable carrier. Pharmaceutical preparations can be preferably present in the form of injectibles, tablets, capsules, syrups, elixirs, ointments, creams, patches, implants, aerosols, sprays and suppositories (rectal, vaginal and urethral). Another aspect of the present invention then relates to a pharmaceutical preparation as prepared according to the invention.

Another aspect of the disclosure relates to the use of at least one biomarker or a set/panel of biomarkers of about 5, about 10, about 20, about 30 of said AHR biomarkers according to Table 1 or at least 10%. at least 20%, at least 30%, at least 40%. at least 50%, at least 60%, at least 70%, at least 80% or at least 90% or more of the genes according to Table 1 for monitoring AHR activation in a biological sample according to the present invention, or for screening for a modulator of AHR activation genes according to the present invention, or for testing the biological safety according to the present invention or for a diagnosis according to the present invention.

In another aspect, the disclosure is directed to a method for screening for a modulator of AHR activation genes, comprising performing the method for determining AHR activation signature, and further comprising contacting at least one candidate modulator compound with said biological sample or modulating the levels of at least one candidate modulator with said biological sample, wherein a change in the biological state of said at least one AHR biomarker of said signature in the presence of said at least one compound compared to the absence of said at least compound identifies a modulator, wherein said modulator is selected from an inhibitor or an agonist of said biological state.

In some embodiments, the modulator is selected from TCDD, FICZ, Kyn, SR1, CH223191, a proteinaceous AHR binding domain, a small molecule, a peptide, a mutated version of a protein, for example an intracellular or recombinantly introduced protein, and a library of said compounds, environmental substances, probiotics, toxins, aerosols. medicines, nutrients, galenic compositions, plant extracts, volatile compounds, homeopathic substances, incense, pharmaceutical drugs, vaccines, i.v. compounds or compound mixtures derived from organisms for example animals, plants, fungi, bacteria, archaea. chemical compounds, and compounds used in food or cosmetic industry.

In some embodiments, the at least one biological state of said at least one AHR biomarker according to Table 1 for said sample is detected using a high-throughput method.

In the methods of the present invention, in general the biomarkers can be detected and/or determined using any suitable assay. Detection is usually directed at the qualitative information (“marker yes-no”), whereas determining involves analysis of the quantity of a marker (e.g. expression level and/or activity). Detection is also directed at identifying mutations that cause altered functions of individual markers. The choice of the assay(s) depends on the parameter of the marker to be determined and/or the detection process.

Thus, the determining and/or detecting can preferably comprise a method selected from subtractive hybridization, microarray analysis, DNA sequencing, qPCR, ELISA, enzymatic activity tests, cell viability assays, for example an MTT assay, phosphoreceptor tyrosine kinase assays, phospho-MAPK arrays and proliferation assays, for example the BrdU assay, proteomics, HPLC and mass spectrometry.

In some embodiments, the methods of the instant disclosure are also amenable to automation, and said activity and/or expression is preferably assessed in an automated and/or high-throughput format. In some embodiments. this involves the use of chips and respective machinery, such as robots.

Another aspect of the present disclosure is directed to a diagnostic kit comprising materials for performing a method according to this disclosure in one or separate containers. In some embodiments, the kit further comprises auxiliary agents and/or instructions for performing said method. The kit may comprise the panel of biomarkers as identified herein or respective advantageous marker sub-panels as discussed herein. Furthermore, included can be dyes, biomarker-specific antibody, and oligos, e.g. for PCR-assays.

In some embodiments, the present disclosure is directed to a panel of biomarkers identified by a method according to the methods of this disclosure. In some embodiments. the present disclosure is directed to use of the panel of biomarkers for monitoring AHR activation in a biological sample, or for screening for a modulator of AHR activation genes.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The following embodiments are part of the invention:

1. A method for determining AHR activation signature for a biological sample, comprising detecting at least one biological state of at least one AHR biomarker according to table 1 for said sample, identifying a change of said biological state of said at least one AHR biomarker compared to a house keeping gene or control biomarker, and assigning said at least one AHR biomarker to said AHR activation signature for said biological sample, if said at least one biomarker provides a significance of said AHR activation signature of p<0.05 at a minimal number of markers in the signature and/or a fold of change of said AHR activation signature of at least about 1.5 at a minimal number of markers in the signature in the case of up-regulation or of at least about 0.67 at a minimal number of markers in the signature in the case of down-regulation.
2. The method according to embodiment 1, wherein said biological sample is selected from a sample comprising biological fluids comprising biomarkers, human cells, tissues, whole blood, cell lines, primary cells, IPCs, hybridomas, recombinant cells, stem cells, and cancer cells, bone cells, cartilage cells, nerve cells, glial cells, epithelial cells, skin cells, scalp cells, lung cells, mucosal cells, muscle cells, skeletal muscles cells, straited muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cell types thereof.
3. The method according to embodiment 1 or 2, wherein said biological state as detected is selected from mutations, nucleic acid methylation, copy numbers, expression, amount of protein, metabolite, and activity of said at least one AHR biomarker.
4. The method according to any one of embodiments 1 to 3, wherein said AHR activation signature provides a significance of p<0.05, preferably of p<0.01, and more preferably of p<0.001, and more preferably p<0.0001 or at least an absolute fold of change of said AHR activation signature of at least about 1.5 in case of up-regulation or at least an absolute fold change of at least about 0.67 in the case of down regulation at a minimal number of markers in the signature.
5. The method according to any one of embodiments 1 to 4, wherein said AHR activation signature comprises about 5, about 10, about 20, about 30 of said AHR biomarkers according to table 1 or at least 10%., at least 20%, at least 300%, at least 40%, at least 50%, at least 60%, at least 70%, at least 80% or at least 90% or more or all of said AHR biomarkers according to table 1.
6. The method according to any one of embodiments 1 to 5, wherein said AHR activation signature is identified in a sample under physiological conditions or under disease conditions, for example, in biological safety screenings, toxicology studies, cancer, autoimmune disorders, degeneration, inflammation and infection, or under stress conditions, for example, biological. mechanical and environmental stresses.
7. The method according to any one of embodiments 1 to 6, wherein said method further comprises the step of using said AHR activation signature for unsupervised clustering or supervised classification of said samples into AHR activation subgroups.
8. The method according to any one of embodiments 1 to 7, wherein said AHR activation signature or AHR activation subgroups are further used to define AHR activation modulated functions, for example, angiogenesis, drug metabolism, external stress response, hemopoiesis, lipid metabolism, cell motility, and immune modulation.
9. A method for monitoring AHR activation in a biological sample in response to at least one compound, comprising performing the method according to any one of embodiments 1 to 8 on samples that have been obtained during the course of contacting said sample with at least one pharmaceutically active compound, toxin or other modulator compound, wherein said modulator is preferably selected from an inhibitor or an agonist of said biological state.
10. A method for screening for a modulator of AHR activation genes, comprising performing the method according to any one of embodiments 1 to 8, and further comprising contacting at least one candidate modulator compound with said biological sample or modulating the levels of at least one candidate modulator with said biological sample, wherein a change in the biological state of said at least one AHR biomarker of said signature in the presence of said at least one compound compared to the absence of said at least compound identifies a modulator, wherein said modulator is preferably selected from an inhibitor or an agonist of said biological state.
11. The method according to any one of embodiments 9 to 10, wherein said modulator is selected from TCDD, FICZ, Kyn, SR1, CH223191, a proteinaceous AHR binding domain, a small molecule, a peptide, a mutated version of a protein, for example an intracellular or recombinantly introduced protein, and a library of said compounds, antibodies, environmental substances, probiotics, toxins, aerosols, medicines, nutrients, galenic compositions, plant extracts, volatile compounds, homeopathic substances, incense, pharmaceutical drugs, vaccines, i.v., compounds or compound mixtures derived from organisms for example animals, plants, fungi, bacteria, archaea, chemical compounds, and compounds used in food or cosmetic industry.
12. The method according to any one of embodiments 1 to 11, wherein said at least one biological state of said at least one AHR biomarker according to table 1 for said sample is detected using a high-throughput method.
13. A diagnostic kit comprising materials for performing a method according to any one of embodiments 1 to 12 in one or separate containers, optionally together with auxiliary agents and/or instructions for performing said method.
14. A panel of biomarkers identified by a method according to any one of embodiments 1 to 8.
15. Use of a panel of biomarkers according to embodiment 14 for monitoring AHR activation in a biological sample according to embodiment 9, or for screening for a modulator of AHR activation genes according to any one of embodiment 10 to 12.

The specific examples listed below are only illustrative and by no means limiting.

### Examples

### Example 1

### Generating the AHR Gene Transcriptional/Activation Signature

First, existing datasets for different AHR activation or inhibition conditions were identified in the GEO database (Edgar R. et al., Nucleic Acids Res.; 2002: 30(1):207-10). The search was performed using an in-house tool using several keywords. The list of datasets was manually curated and a cutoff for differentially expressed genes was set at log 2 fold change of 0.3 (and an adjusted p-value threshold of 0.05). In addition, AHR targets were retrieved from the Transcription Factor Target Gene Database (Plaisier C L, et al. Causal Mechanistic Regulatory Network for Glioblastoma Deciphered Using Systems Genetics Network Analysis. Cell Syst. 2016 August; 3(2):172-86) and merged with the gene list curated from the GEO search.

A semantic analysis was carried out to correctly identify the appearance of gene names including AHR in given freely available free texts with GeNo (Wermter, J., Tomanek, K. & Hahn, U. High-performance gene name normalization with GeNo. Bioinformatics 25, 815-821 (2009)) and gene interactions, called events, using BioSem (Bui, Q.-C. & Sloot, P. M. A.: A robust approach to extract biomedical events from literature.; Bioinformatics; 28, 2654-2661 (2012)). The output of BioSem was then stored in an ElasticSearch index (Elastic webpage). From this index, event items referencing AHR as an interaction member with a regulation event were selected. Results were manually curated to obtain the final list of literature mentioning AHR associated interaction events. Human orthologues were used to replace mouse genes in the NLP search results. The gene annotations of both text mining and dataset searches results were harmonized by cross referencing with the accepted HGNC symbols (HGNC website) as per the hg38 reference. Genes overlapping between the two lists were used to constitute the core AHR activation signature consisting of 166 genes (Table 1).

### Annotation of the AHR Gene Transcriptional/Activation Signature

Gene ontology analysis of the core AHR activation signature was performed using the clusterProfiler package (Yu, Guangchuang, et al. 2012. “ClusterProfiler: An R Package for Comparing Biological Themes Among Gene Clusters.” OMICS: A Journal of Integrative Biology 16 (5):284-87), applying the method described by Boyle et al. (2004) (Boyle, Elizabeth I. et al. 2004. “GO: TermFinder-open Source Software for Accessing Gene Ontology Information and Finding Significantly Enriched Gene Ontology Terms Associated with a List of Genes.” Bioinformatics (Oxford, England) 20 (18):3710-5). Bonferroni correction was used to control for multiple testing and a p-value cutoff of 0.01 was used for selecting enriched ontology terms. The semantic similarity algorithm GOsemsim (Yu, Guangchuang. et al. 2010. “GOSemSim: An R Package for Measuring Semantic Similarity Among Go Terms and Gene Products.” Bioinformatics 26 (7):976-78) was used for grouping of ontology terms followed by filtering of higher/general levels ontology term. The remaining ontology terms were categorized into eight groups descriptive of AHR activation mediated biological processes.

### Microarray and RNA-Seq Data Analysis

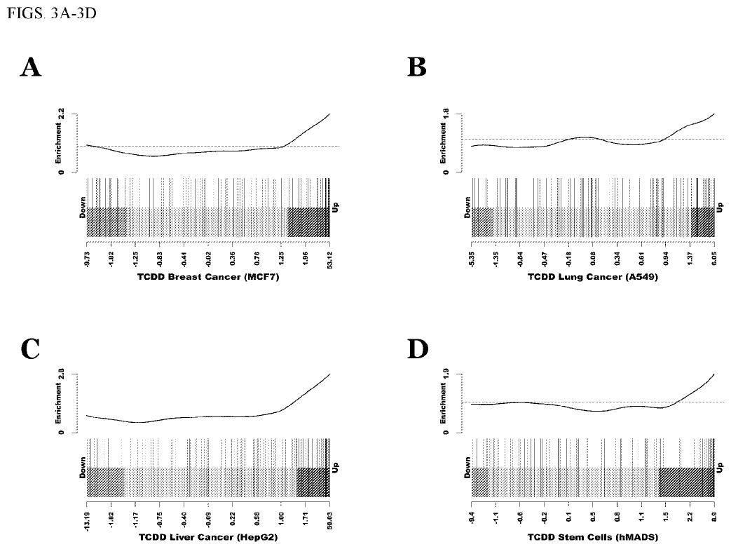

Additional datasets, not used in defining the AHR biomarker set of Table 1, were used for validation (FIGS. 3-6). The datasets comprised microarrays from multiple platforms (Affymetrix, Illumina and Agilent) and RNAseq. Datasets of 32 cancer types from the Cancer Genome Atlas (TCGA) comprising RNAseq and reverse phase protein arrays (RPPA) were used for defining cancer and cancer subgroup specific AHR-signature genes, the transition of the AHR signature from the transcriptional layer (RNA expression) to the protein layer (RPPA), the consistency in defining AHR functional groups and outcomes when applying different methods for unsupervised clustering, and when patients are grouped according to clinical outcome.

Array datasets—The Affiymetrix microarray chips “human gene 2.0 ST” were analyzed using the oligo package and annotated using NetAffx (Carvalho, B.; et al. Exploration, Normalization. and Genotype Calls of High Density Oligonucleotide SNP Array Data. Biostatistics, 2006). Other Affymetrix chips were analyzed using the Affy and Affycoretools packages. Raw CEL files were imported from disk or downloaded from Gene Expression Omnibus (GEO) using GEOquery (Davis S, Meltzer P (2007). “GEOquery: a bridge between the Gene Expression Omnibus (GEO) and BioConductor.” Bioinformatics, 14, 1846-1847), followed by RMA normalized and summarization. Illumina and Agilent array datasets were analyzed using lumi (Du, P., Kibbe, W. A. and Lin, S. M., (2008) ‘lumi: a pipeline for processing Illumina microarray’, Bioinformatics 24(13):1547-1548; and Lin, S. M., Du, P., Kibbe, W. A., (2008) ‘Model-based Variance-stabilizing Transformation for Illumina Microarray Data’, Nucleic Acids Res. 36, e11) and limma (Ritchie, M E, et al. (2015). limma powers differential expression analyses for RNA-sequencing and microarray studies. Nucleic Acids Research 43(7), e47).

RNA-seq datasets—Raw counts and metadata were downloaded from GEO using GEOquery and saved as a DGElist (Robinson, M D, McCarthy, D J, Smyth, G K (2010). edgeR: a Bioconductor package for differential expression analysis of digital gene expression data. Bioinformatics 26, 139-140). The harmonized HT-Seq counts of TCGA datasets were downloaded using TCGAbiolinks (Colaprico A, wt al. (2015). “TCGAbiolinks: An R/Bioconductor package for integrative analysis of TCGA data.” Nucleic Acids Research. doi: 10.1093/nar/gkv1507) from GDC (the NIH GDC website), and only patients with the identifier “primary solid tumor” were retained, with the exception of melanoma that was split into datasets for primary and advanced melanoma cohorts. Genes with less than 10 counts were filtered followed by TMM normalization (Robinson, M D, and Oshlack, A (2010). A scaling normalization method for differential expression analysis of RNA-seq data. Genome Biology11, R25) and variance modelling using voom (Robinson, M D, and Oshlack, A (2010). A scaling normalization method for differential expression analysis of RNA-seq data. Genome Biology 11, R25).

RPPA datasets—Level 4 standardized data was downloaded from The Cancer Proteome Atlas (TCPA) (the TCPA website). The patient datasets were reduced to the overlap between both RPPA and RNAseq data sets.

### Differential Gene Expression and Gene Set Testing

The eBayes adjusted moderated t-statistic was applied for differential gene expression using limma (Ritchie, M E, et al. (2015). limma powers differential expression analyses for RNA-sequencing and microarray studies. Nucleic Acids Research 43(7), e47) and limma-trend (Phipson, B, et al. (2016). Robust hyperparameter estimation protects against hypervariable genes and improves power to detect differential expression. Annals of Applied Statistics 10(2), 946-963) or the limma RNA-seq pipeline (Ritchie, M E, et al. (2015). limma powers differential expression analyses for RNA-sequencing and microarray studies. Nucleic Acids Research 43(7), e47). Batch effects, when present, were accounted for in the linear regression. Gene set testing of AHR activation was performed using roast (Wu, D., et al. (2010). ROAST: rotation gene set tests for complex microarray experiments. Bioinformatics 26, 2176-2182).

Association of AHR Activation with Patient Groups of Median Separated Enzyme Expression

Assessing the association of AHR activation with Trp degrading enzymes, TCGA patients were divided by the median into groups of high or low expression of IDO1 or TDO2, and differential gene expression and gene set testing was conducted as described above.

### Generating AHR Activation Score

Using the AHR signature, the single sample gene set enrichment scores was estimated using the GSVA package (Htnzelmann S, Castelo R, Guinney J (2013). “GSVA: gene set variation analysis for microarray and RNA-Seq data.” BMC Bioinformatics, 14, 7), the inventors refer to as the AHR activation score. This score is used for defining gene co-expression networks representing AHR functional outcomes, and for comparing the status of AHR modulation in patients of different clinical subtypes.

Gene Correlation Networks Associated with AHR Activation

The normalized and voomed DGEList of publicly available GEO data was used for weighted gene co-expression network analysis (WGCNA) (Langfelder P, Horvath S (2008) WGCNA: an R package for weighted correlation network analysis. BMC Bioinformatics 2008, 9:559). Soft thresholds were estimated for signed hybrid networks in single block settings. Adjacency and topology overlapping matrices were calculated using bi-correlation matrices and Eigen genes representing the first principle components of each module were returned. Selecting WGCNA modules associated with AHR activation was conducted by performing a global test (Goeman, J. J., van de Geer, S. A., de Kort, F., and van Houwelingen, J. C. (2004). A global test for groups of genes: testing association with a clinical outcome. Bioinformatics, 20(1):93-99; Goeman, J. J., van de Geer, S. A., and van Houwelingen, J. C. (2006). Testing against a high-dimensional alternative. Journal of the Royal Statistical Society Series B Statistical Methodology, 68(3):477-493: and Goeman, J. and Finos, L. (2012). The inheritance procedure: multiple testing of tree-structured hypotheses. Statistical Applications in Genetics and Molecular Biology, 11(1):1-18)) using the AHR activation score as the response and the WGCNA modules as model predictors. Additionally, using Pearson correlation, as implemented in the Hmisc package (Harrell Miscellaneous webpage from R Archive Network), AHR activation scores were correlated with WGCNA modules. Modules that overlapped the global test and Pearson correlation results, with a p-value of 0.05 or less in both tests, were selected as the AHR associated modules, regardless of the direction of association, i.e. both positively and negatively associated modules were retained if overlapping and satisfying the p-value cutoff.

### Defining AHR Activation Sub-Groups

K-means consensus clustering (Monti, S., et al. (2003); Machine Learning, 52, 91-118. and Wilkerson, D. M, Hayes, Neil D (2010). “ConsensusClusterPlus: a class discovery tool with confidence assessments and item tracking.” Bioinformatics, 26(12), 1572-1573), using AHR associated modules, was performed to define patient subgroups with AHR activation. The number of clusters for each tumor type was assessed using consensus heatmaps, cumulative distribution function plots, elbow plots and samples' cluster identities. The values of K explored were 2-20, with k=2-4 providing the most stable clusters. The group separation was further examined using principle component analysis.

### Cell Culture

U-87MG were obtained from ATCC. U-87MG were cultured in phenol red-free, high glucose DMEM medium (Gibco, 31053028) supplemented with 10% FBS (Gibco, 10270106), 2 mM L-glutamine, 1 mM sodium pyruvate, 10 U/mL penicillin and 100 pg/mL streptomycin (referred to as complete DMEM). Cell lines were cultured at 37° C. and 5% CO₂. Cell lines were authenticated and certified to be free of mycoplasma contamination.

### Cell Culture Treatment Conditions

For treatment of adherent cells with AHR ligands, 4×10⁵ cells per well were seeded in six well plates and incubated for 24 h prior to treatment. Non-adherent cells were seeded at 5×10⁵ cells/mL in 24 well plates and treated immediately. For verification of the generated AHR signature, cells were treated with the established AHR agonists TCDD (10 nM, American Radiolabeled Chemicals Inc.,), FICZ (100 nM, Cayman Chemicals, 19529), Kyn (50 μM, Sigma Aldrich), KynA (50 uM, Sigma-Aldrich, K3375) and indole-3-carboxaldehyde (6.25 μM to 100 μM, Sigma-Aldrich, 129445) for 24 h.

### Stable Knockdown of U-87MG Cells

Stable knockdown of AHR in U-87MG cells was achieved using shERWOOD UltramiR Lentiviral shRNA targeting AHR (transOMIC Technologies, TLHSU1400-196-GVO-TRI). Glioma cells were infected with viral supematants containing either shAHR or shControl (shC) sequences to generate stable cell lines. Both shAHR sequences displayed similar knockdown efficiency and stable cell lines with shAHR #1 were used for experiments.

shERWOOD UltramiR shRNA sequences are:

shAHR#1 (ULTRA-3234821):
(SEQ ID NO: 1)
5′-TGCTGTTGACAGTGAGCGCAGGAAGAATTGTTTTAGGATATAGTGA
AGCCACAGATGTATATCCTAAAACAATTCTTCCTTTGCCTACTGCCTCG
GA-3′;
shAHR#2 (ULTRA-3234823):
(SEQ ID NO: 2)
5′-TGCTGTTGACAGTGAGCGCCCCACAAGATGTTATTAATAATAGTGA
AGCCACAGATGTATTATTAATAACATCTTGTGGGATGCCTACTGCCTCG
GA-3′;
shC (ULTRA-NT#4):
(SEQ ID NO: 3)
5′-TGCTGTTGACAGTGAGCGAAGGCAGAAGTATGCAAAGCATTAGTGA
AGCCACAGATGTAATGCTTTGCATACTTCTGCCTGTGCCTACTGCCTCG
GA-3′.

### RNA Isolation and Real Time PCR

Total RNA was harvested from cultured cells using the RNeasy Mini Kit (Qiagen) followed by cDNA synthesis using the High Capacity cDNA reverse transcriptase kit (Applied Biosystems). StepOne Plus real-time PCR system (Applied Biosystems) was used to perform real time PCR of cDNA samples using SYBR Select Master mix (Thermo Scientific). Data was processed and analysed using the StepOne Software v 2.3. Relative quantification of target genes was done against RNA18S as reference gene using the 2^{ΔΔCt} method. Human primer sequences are,

18S RNA-Fwd
(SEQ ID NO: 4)
5′-GATGGGCGGCGGAAAATAG-3′,
18S RNA-Rev
(SEQ ID NO: 5)
5′-GCGTGGATTCTGCATAATGGT-3′,
IL1B-Fwd
(SEQ ID NO: 6)
5′-CTCGCCAGTGAAATGATGGCT-3′,
IL1B-Rev
(SEQ ID NO: 7)
5′-GTCGGAGATTCGTAGCTGGAT-3′,
CYP1B1-Fwd
(SEQ ID NO: 8)
5′-GACGCCTTTATCCTCTCTGCG-3′,
CYP1B1-Rev
(SEQ ID NO: 9)
5′-ACGACCTGATCCAATTCTGCCCA-3′,
EREG-Fwd
(SEQ ID NO: 10)
5′-CTGCCTGGGTTTCCATCTTCT-3′,
EREG-Rev
(SEQ ID NO: 11)
5′-GCCATTCATGTCAGAGCTACACT-3′,
NPTX1-Fwd
(SEQ ID NO: 12)
5′-CATCAATGACAAGGTGGCCAAG-3′,
NPTX1-Rev
(SEQ ID NO: 13)
5′-GGGCTTGATGGGGTGATAGG-3′,
SERPINEB2-Fwd
(SEQ ID NO: 14)
5′-ACCCCCATGACTCCAGAGAA-3′,
SERPINEB2-Rev
(SEQ ID NO: 15)
5′-CTTGTGCCTGCAAAATCGCAT-3′,
TIPARP-Fwd
(SEQ ID NO: 16)
5′-CACCCTCTAGCAATGTCAACTC-3′,
TIPARP-Rev
(SEQ ID NO: 17)
5′-CAGACTCGGGATACTCTCTCC-3′,
MMP1-Fwd
(SEQ ID NO: 18)
5′-GCTAACCTTTGATGCTATAACTACGA-3′,
MMP1-Rev
(SEQ ID NO: 19)
5′-TTTGTGCGCATGTAGAATCTG-3′,
AHRR-Fwd
(SEQ ID NO: 20)
5′-CCCTCCTCAGGTGGTGTTTG-3′,
AHRR-Rev
(SEQ ID NO: 21)
5′-CGACAAATGAAGCAGCGTGT-3′,
ABCG2-Fwd
(SEQ ID NO: 22)
5′-TTCCACGATATGGATTTACGG-3′,
ABCG2-Rev
(SEQ ID NO: 23)
5′-GTTTCCTGTTGCATTGAGTCC-3′,
EGR1-Fwd
(SEQ ID NO: 24)
5′-CTGACCGCAGAGTCTTTTCCT-3′,
and
EGR1-Rev
(SEQ ID NO: 25)
5′-GAGTGGTTTGGCTGGGGTAA-3′,

### Software and Statistics

Graphical and statistical analysis of gene (real time-PCR) was done using GraphPad Prism software versions 6.0 and 8.0. Unless otherwise indicated, data represents the mean±S.E.M of at least 3 independent experiments. In cases where data was expressed as absolute fold of change, these values were Log₁₀ transformed and the resulting values were used for statistical analysis. Depending on the data, the following statistical analyses were applied: two-tailed student's t-test (paired or unpaired) and repeated measures ANOVA with Dunnett's multiple comparisons test. Significant differences were reported as *p<0.05, **p<0.01, ***p<0.001, ****p<0.0001. NS indicates no significant difference. For bioinformatics analysis, unless stated otherwise, all pairwise comparisons were performed using Kruskal-Wallis and Wilcoxon rank sum test, and all reported p-values were adjusted using the Benjamini-Hochberg procedure.

### Example 2: Validation of AHR Activation Signal and Association of AHR Activation with Patient Groups of Median Separated Enzyme Expression

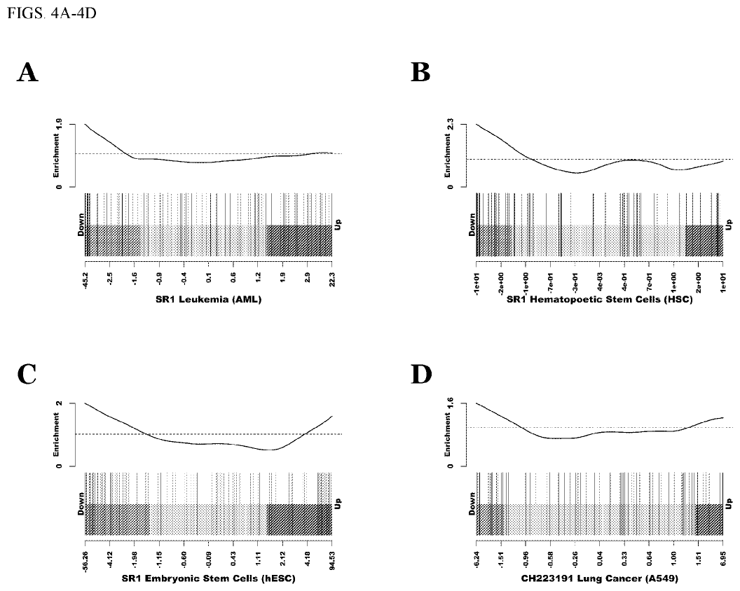

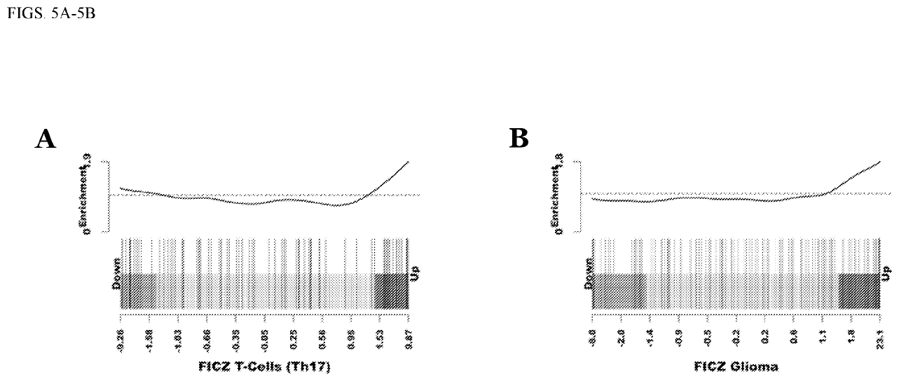

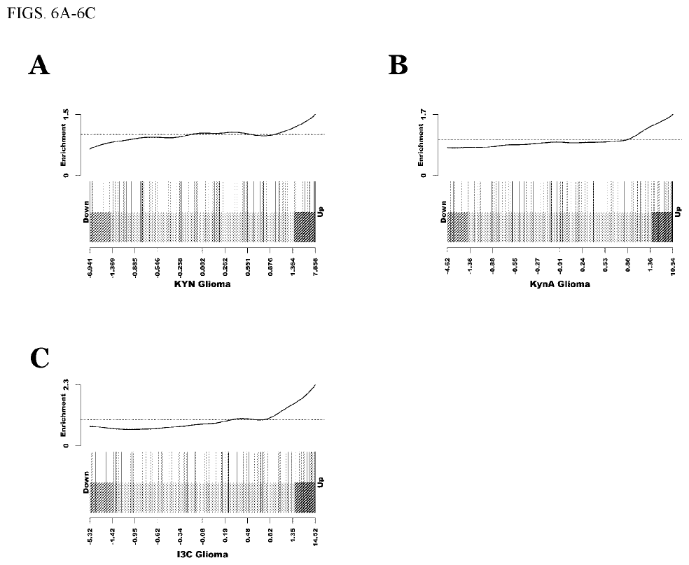

The AHR signature was validated using roast gene set enrichment in distinct datasets of cells treated with TCDD (FIG. 3), the AHR inhibitors SR1 (FIG. 4A-4C), or CH223191 (FIG. 4D), as well as the endogenous AHR agonists 6-formylindolo(3,2b)carbazole (FICZ) and kynurenine, kynurenic acid and indole-3-carboxaldehyde (FIG. 5 and FIG. 6).

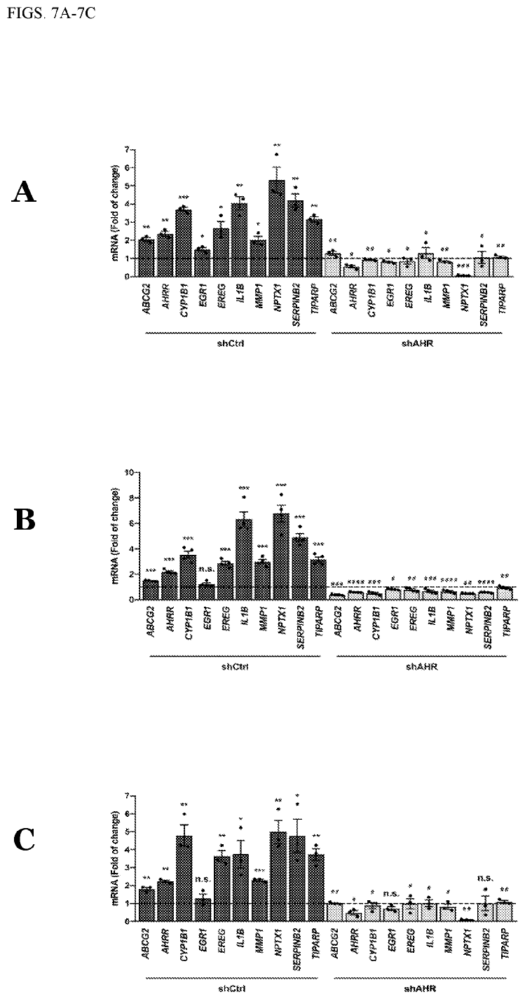

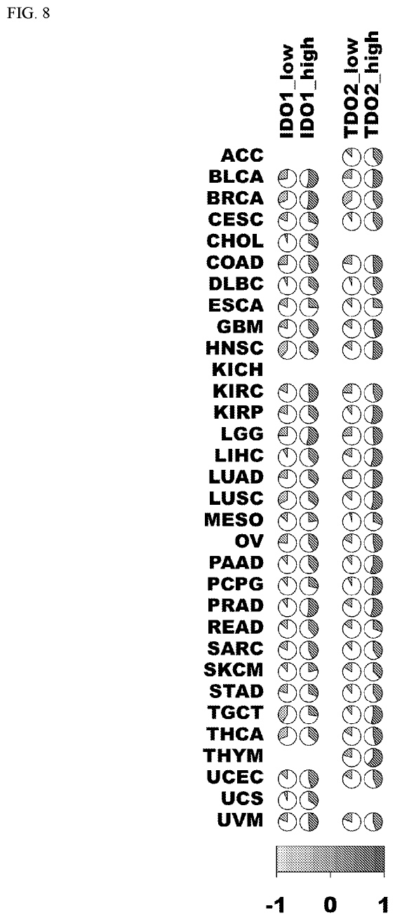

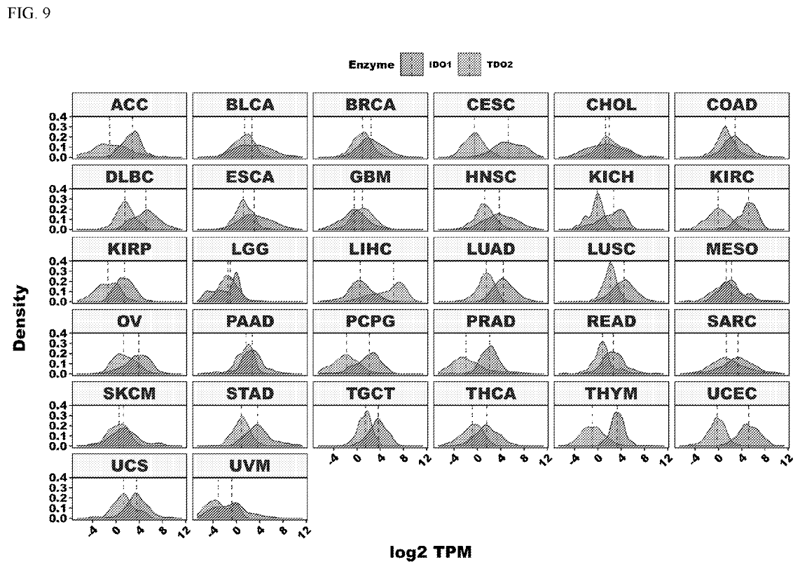

In addition, the inventors performed qRT-PCRs of selected signature genes in conditions of AHR activation with TCDD, FICZ or Kyn as well as combined ligand activation and AHR knockdown (FIG. 7). Owing to the cell/tissue and ligand specificity of AHR target gene expression, the inventors confirmed that the AHR signature is able to detect modulation of AHR activity also in cell types (FIG. 3D: FIG. 4A-4C: FIG. 5: FIG. 6) and in response to ligands (FIG. 4D, FIG. 5, FIG. 6 and FIG. 7B-7C) that were not employed to generate the AHR signature. The AHR-signature was used to evaluate the association of AHR activity in tumor tissue and the expression levels of IDO1 and TDO2, the two key rate limiting enzymes in the catabolism of Trp to Kyn. Of note, it has been reported that the level of Kyn production in TCGA tumors is reflected by the expression of the genes along the Trp pathway 338. This in turn means that the expression of IDO1 and TDO2, the rate limiting enzymes of Trp degradation leading to Kyn production. should be associated with AHR activity. TCGA tumors were divided by the median expression of IDO1 or TDO2 into groups of high or low expression and the AHR-scores was used to test the state of AHR activity when comparing the high to the low expression groups. The AHR signature was significantly upregulated in tumors with high expression of either IDO1 or TDO2, thus reflecting an increase in AHR activity (FIG. 8). However, the association with IDO1 and TDO2 expression didn't explain if the increase in the AHR activity was due to the high expression of IDO1, TDO2 or both. This was due to the overlap of the multimodal distributions of IDO1 and TDO2 expression in the 32 TCGA tumors (FIG. 9).

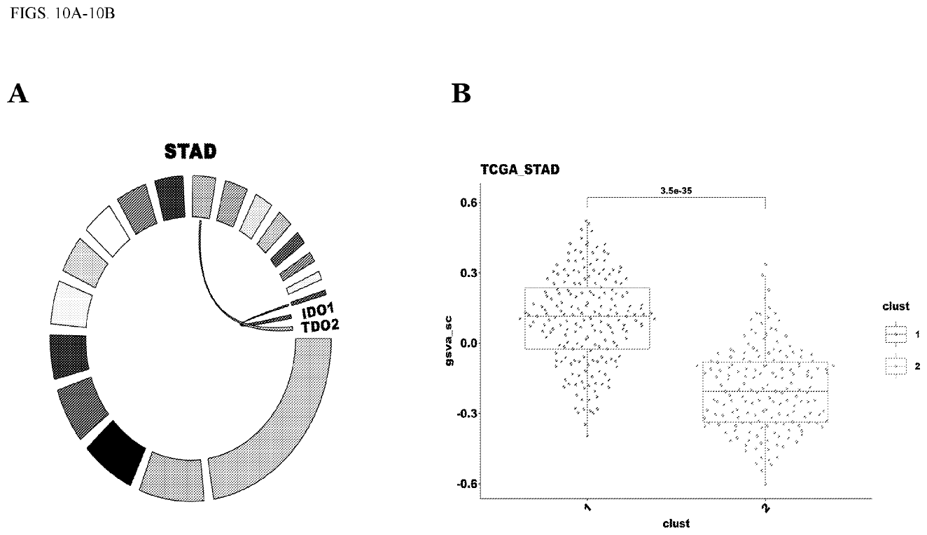

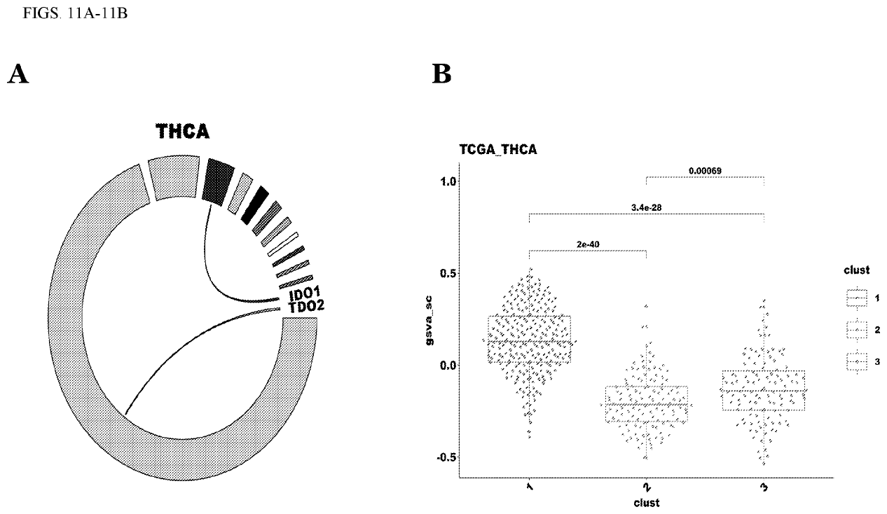

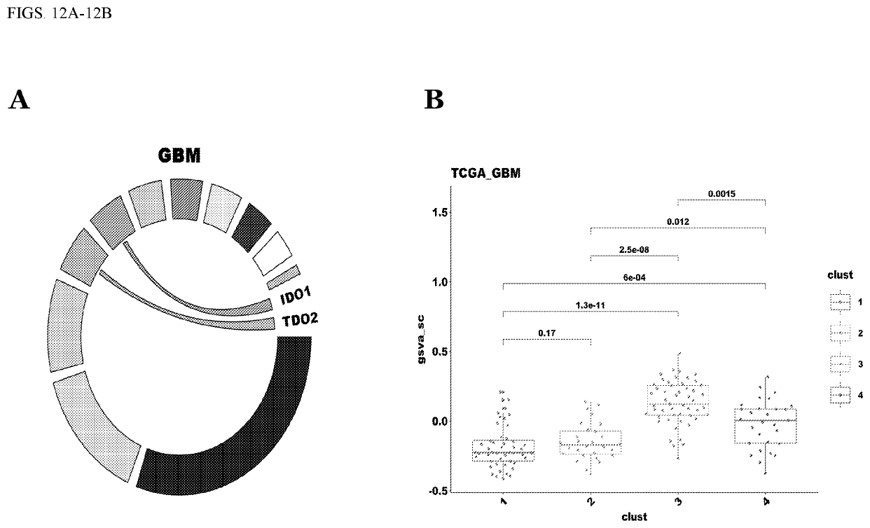

To assess the relative contribution of IDO1 and TDO2 to the AHR activity detected by the AHR-signature, the inventors performed a weighted gene co-expression network analysis (WGCNA) across the 32 TCGA tumor entities. The association between AHR activity (denoted by the AHR-score) and the WGCNA modules was tested to determine which modules show positive or negative associations with the AHR-score (as previously described). The relative contribution of IDO1 and TDO2 to AHR activity was assessed by inspecting the incidence of either of the two enzymes in the positive AAMs (FIGS. 10A, 11A and 12A).

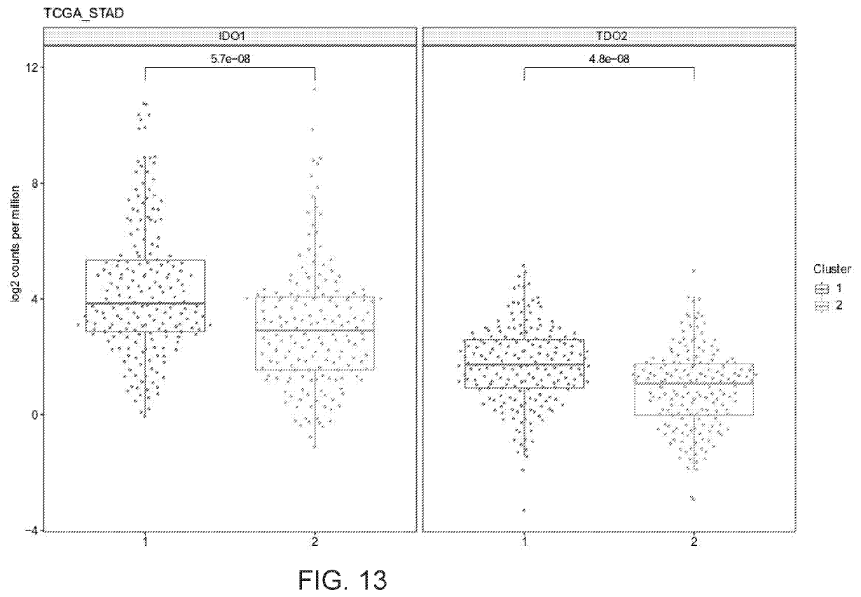

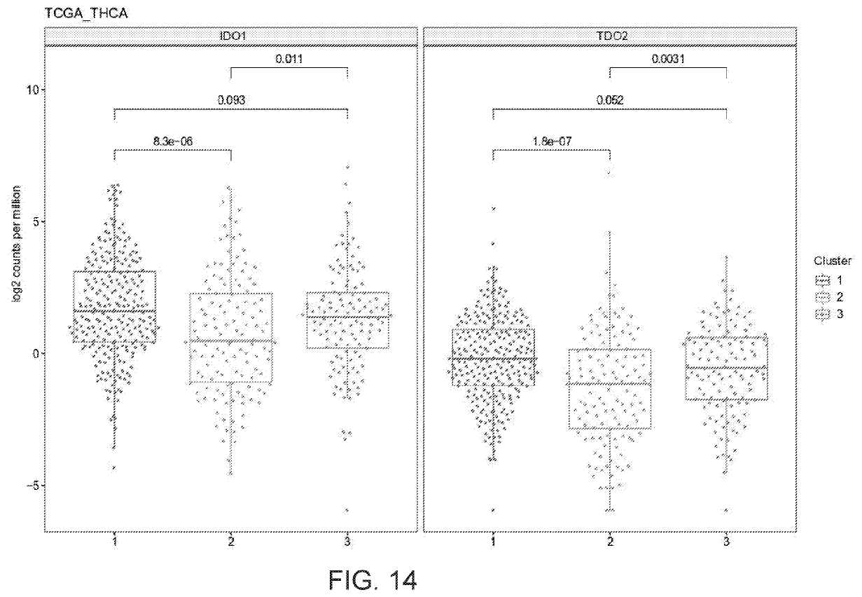

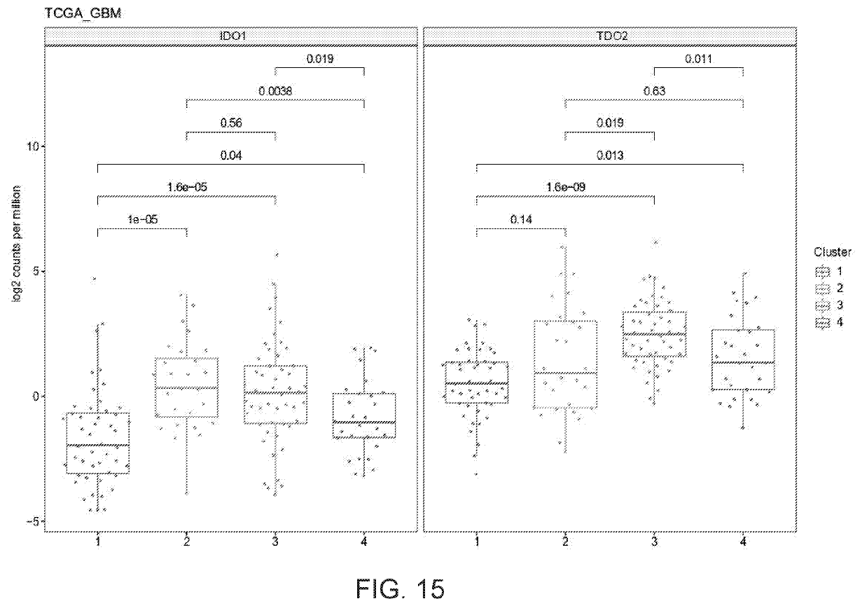

In three different cancer examples that were clustered using the AAMs, the groups that had high AHR-scores (FIGS. 10B, 11B and 12B) showed higher IDO1 expression (FIG. 13), similar IDO1 and TDO2 expression (FIG. 14) or higher TDO2 expression (FIG. 15).

### The Clinical Outcome of Defined AHR Activation Sub-Groups

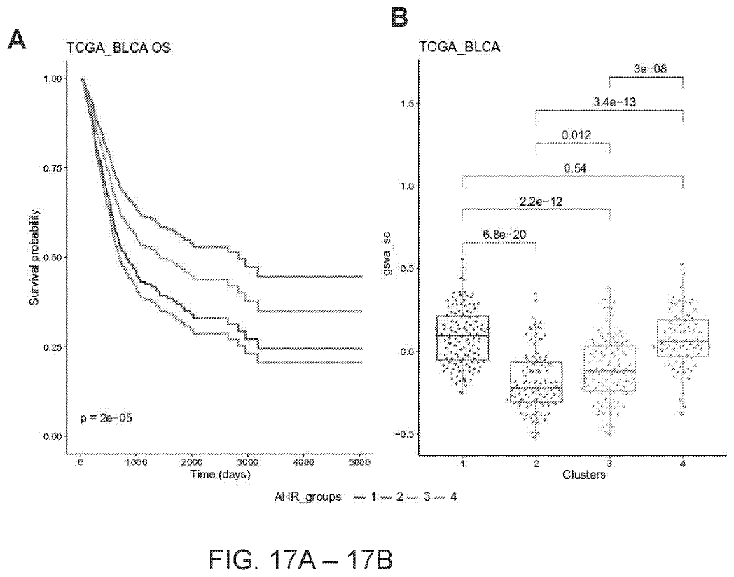

The survival difference between the groups was estimated by fitting a multivariate age-adjusted cox proportional hazard model. Kaplan-Meier curves were used for visualizing the fitted cox proportional hazard models. The AHR defined sub-groups showed significant differences in overall survival outcome (FIGS. 17A-17B).

### Defining the Functional Outcome of AHR Activation Subgroups

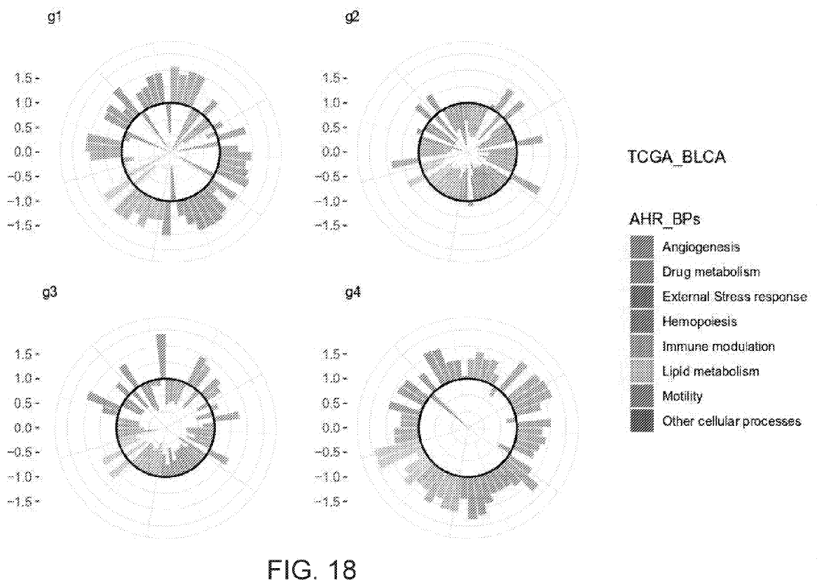

The AHR signature genes are grouped into 56 gene ontology terms according to the biological process representing different AHR biological functions, (these smaller gene groups are denoted AHR-GOs). By using analytic rank based enrichment (PMID: 27322546). the biological process activity (BPA) normalized enrichment score (PMID: 31653878) for each tumor sample was estimated and then the scores were compared between the AHR sub-groups for each cancer. The AHR-GOs BPA scores were averaged for each AHR-subgroup per cancer type and a circular barplot per group was generated (FIG. 18). The examples showed that the higher BPA-scores were proportionate to the level of AHR activation detected by the AHR-score (FIG. 18).

### Example 3: Defining AHR Signature Subsets for Different Cancer Subtypes

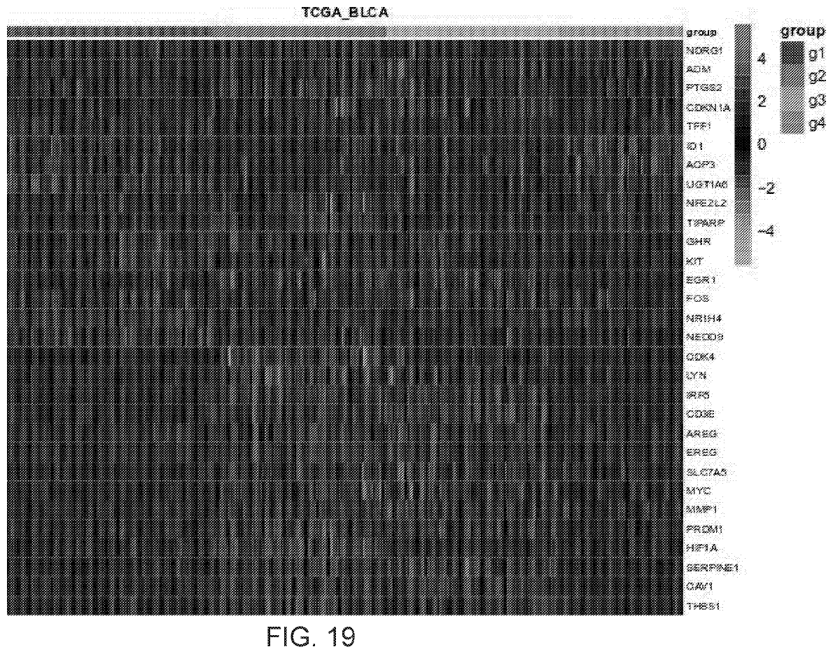

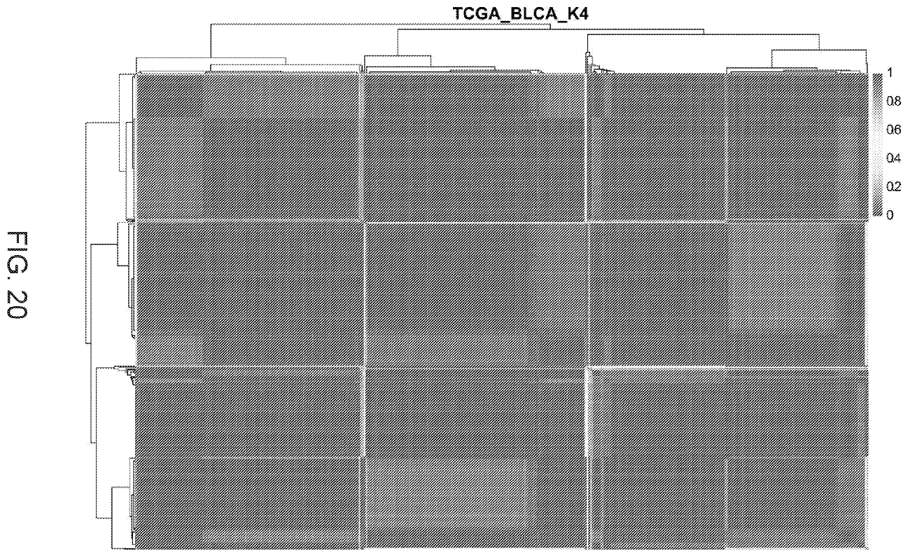

To define subsets of the AHR signature that could be used for each of the 32 cancer types and subsequently cancer sub-groups we constructed prediction models for the AHR-scores using the least absolute shrinkage and selection operator (lasso) method and a random forest based model of recursive feature elimination (RFE). Lasso is a regularized regression method that applies a penalty to the residual sum of squares of predictors leading to the shrinkage of their coefficients, which leads to decreasing the variance and improving the accuracy of the model. The tuning parameter of the lasso is termed lambda, which was determined by cross validation. RFE was ran using random forest control functions and cross validation was performed using the leave on out (LOOCV). Random forest models using all AHR signature genes were created and feature selection was made based on the root mean squared error (RMSE) of the models. The overlap between the lasso and RFE results comprise the least number of AHR signature genes required for calling AHR activation for the different cancer types (Table 2). Furthermore, these AHR signature subsets were evaluated across the cancer sub-groups identified (FIG. 19). Differential gene regulation between the AHR subgroups for every cancer type was used to define the AHR signature gene subsets that are subgroup specific for AHR activity with a defined AHR functional outcome (FIG. 20, Table 3).

### Defining AHR Subgroups Using Non Negative Matrix Factorization (NMF)

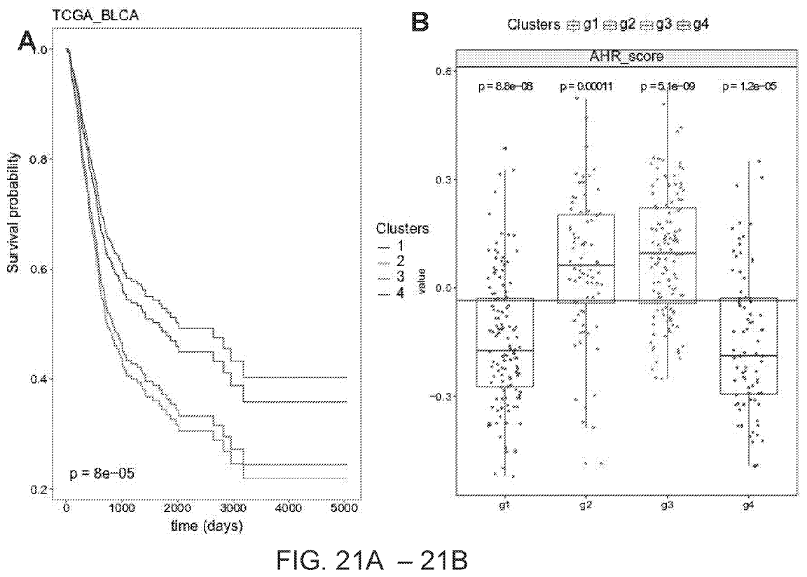

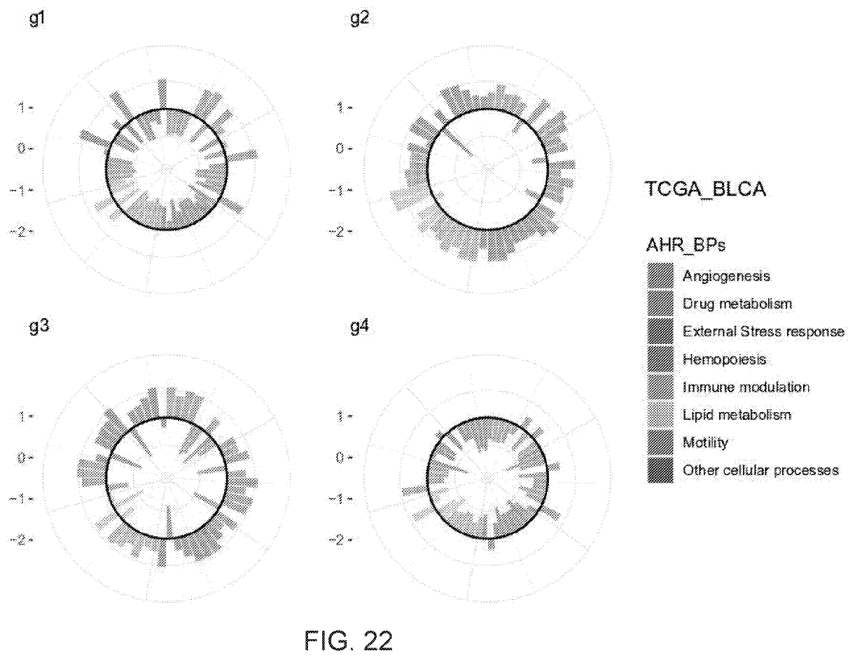

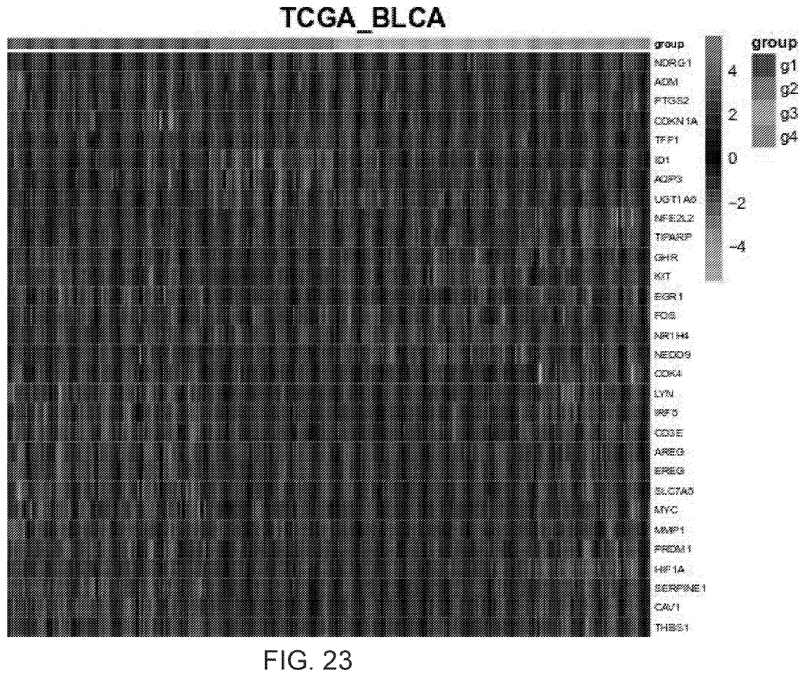

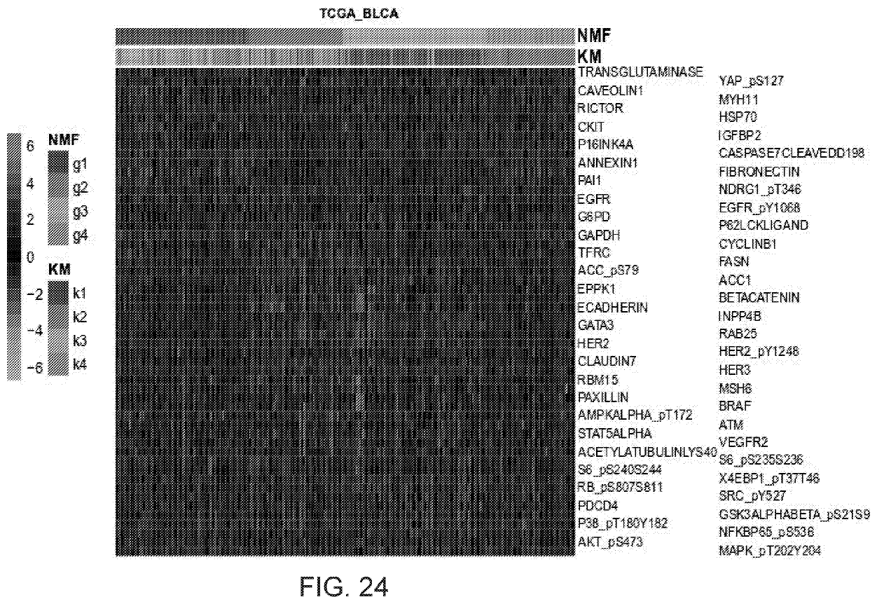

Consensus NMF was applied by using the AAMs previously defined. NMF is a matrix factorization method that constrains the matrix to include only positive values and decomposes the feature matrix into two matrices W and H, which can be used to approximate the original matrix by finding Wand H whose sum of linear combinations (weighted sum of bases) minimizes an error function. The cluster identity is represented by H. The clustering results were determined by evaluating the consensus heatmaps, consensus silhouette coefficient, cophenetic index, sparseness coefficient, and dispersion (FIGS. 23-24). Using Fischer's exact test and the Chi-square test showed that the NMF clustering outcome was significantly similar to the previous clustering results. Although the number of clusters for some cancer types were higher in the NMF clustering result, detailed inspection of group clinical outcome (FIGS. 21A-21B) and the functional outcomes of AHR activation in these groups (FIG. 22) showed that the difference in some of the NMF clustering outcomes was a higher level of granularity of the AHR cancer subgroups. Differential gene expression of the NMF groups for the 32 cancers showed the high level of consistency in defining the AHR signature genes for the different cancer subgroups across all 32 cancers (FIG. 23, Table 4).

### Example 4: Transferring AHR Specific Marker Detection to the Proteomic Layers

RPPA data of tumor samples were grouped according to class assignments of the AHR cancer subtypes from the different clustering solutions described above. RPPA features were filtered to the top 20% showing the highest variation across the different tumors. By comparing the differential regulation of these features across the AHR subgroups for each cancer, we defined RPPA features that could be used for calling AHR activity in both a cancer specific and cancer sub-group specific manner (FIG. 24 and Table 5).

### Grouping Cancer Patients Based on Clinical Features and Analysis of Clinical Outcomes

Tumors are increasingly sub-classified based on molecular characteristics known to affect prognosis and therapy response. To obtain even higher granularity it is important to analyze AHR activity in tumor subgroups with specific clinical characteristics. Using the AHR signature and the methods described above, the inventors analyzed and compared clinically defined subgroups of prevalent cancer entities. of which the inventors show examples of AHR activity and clinical outcomes:

### Non Small Cell Lung Cancer (NSCLC)

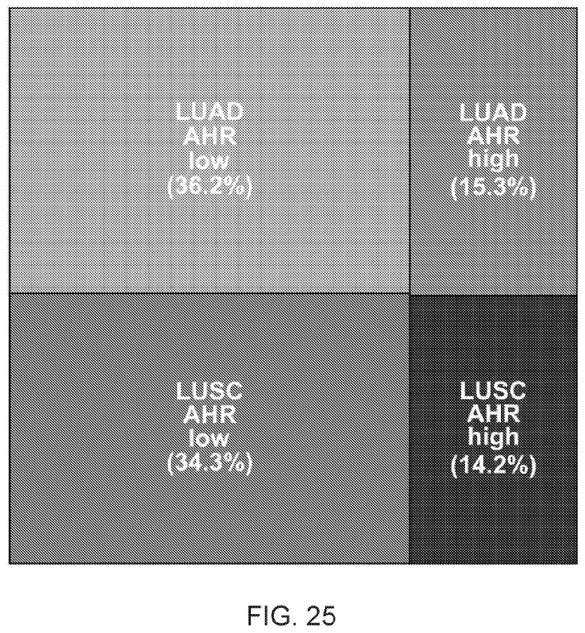

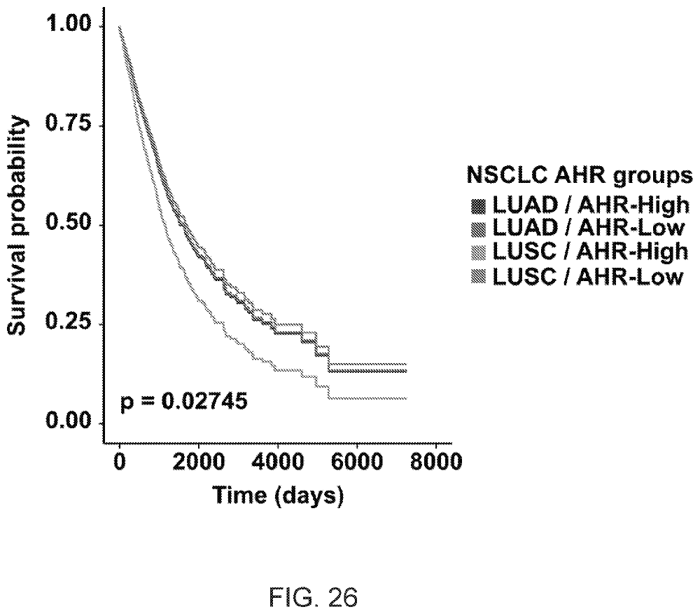

Comparison of AHR activity in the histology subtypes Lung Adenocarcinoma (LUAD) versus Lung Squamous Cell Carcinoma (LUSC) revealed a similar distribution of AHR high and low patients in each histological subtype (FIG. 25). However, analysis of overall survival in these patient groups demonstrated that AHR activity affects overall survival of LUSC but not LUAD patients (FIG. 26).

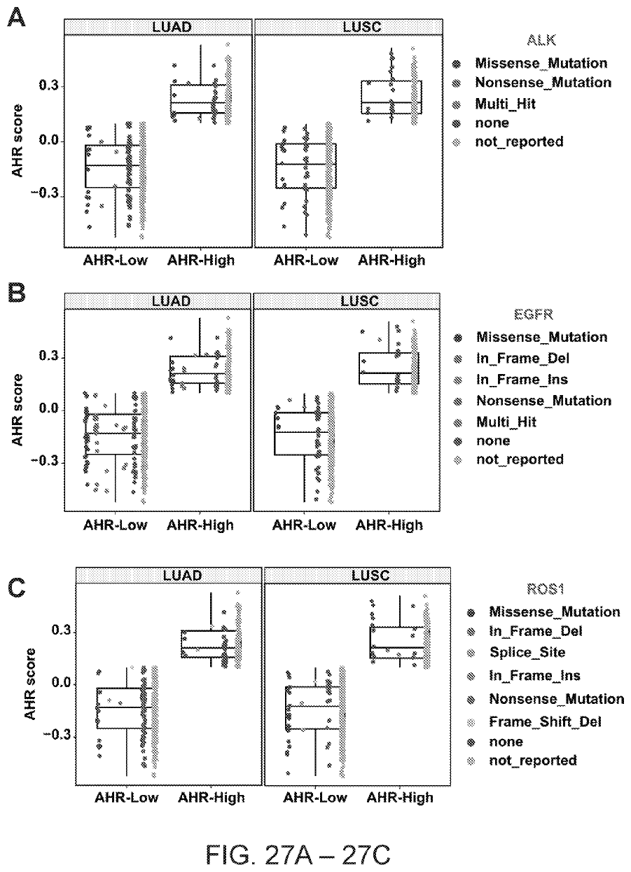

Comparison of NSCLC patients with EGFR activating mutations or ALK/ROS1 rearrangement versus a cohort with no mutation/rearrangement by means of the AHR signature revealed that neither EGFR nor ALK mutations differ between AHR high and low groups (FIGS. 27A and 27B).

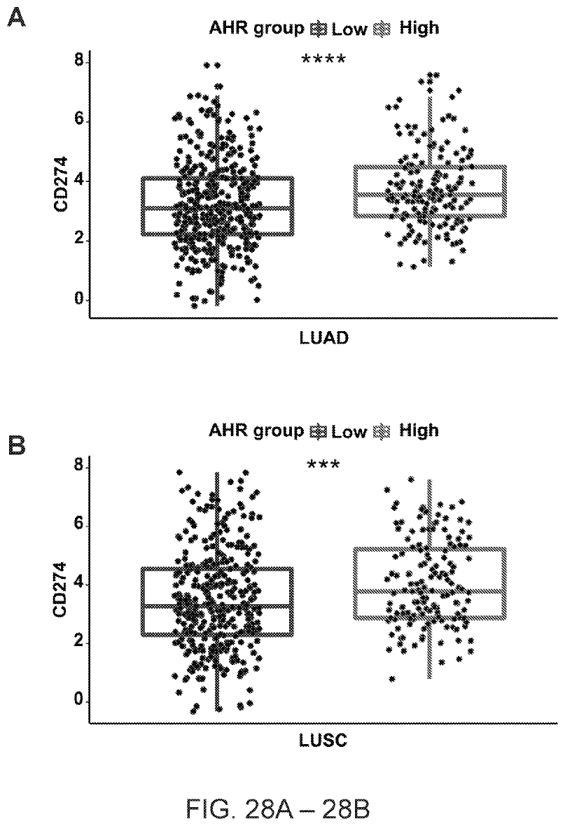

Analysis of PDL-1 (CD274) expression in LUAD and LUSC with high versus low AHR activity revealed increased expression of PDL-1 expression in the AHR high groups (FIG. 28A-28B).

### Head and Neck Squamous Cell Carcinoma (HNSCC)

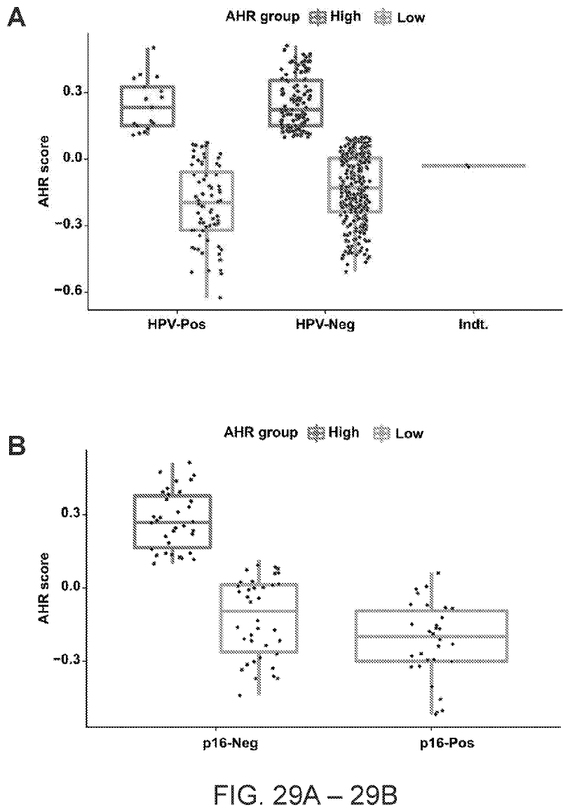

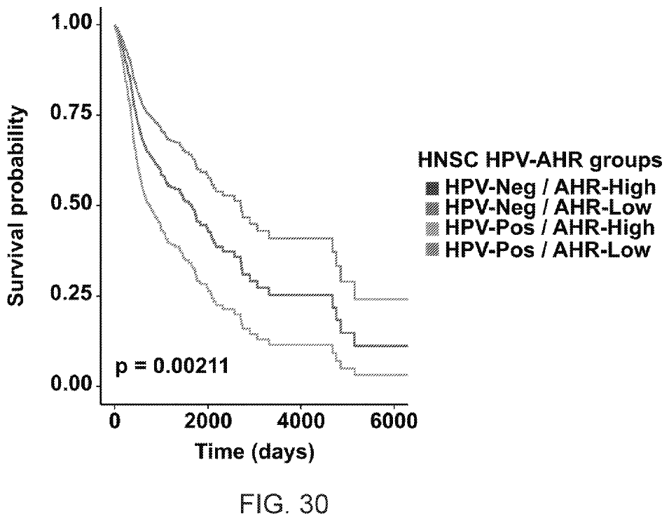

Analysis of human Papilloma Virus (HPV) positive versus HPV negative HNSCC based on either the clinical annotation or p16 expression, revealed similar distributions of AHR high and AHR low groups among HPV positive and negative tumors (FIG. 29A-29B). However, while AHR activity status did not associate with differences in clinical outcome in HPV negative tumors, high AHR activity associated with reduced survival in HPV positive tumors (FIG. 30).

### Example 5: Detecting AHR Activity in Response to Different AHR Modulators

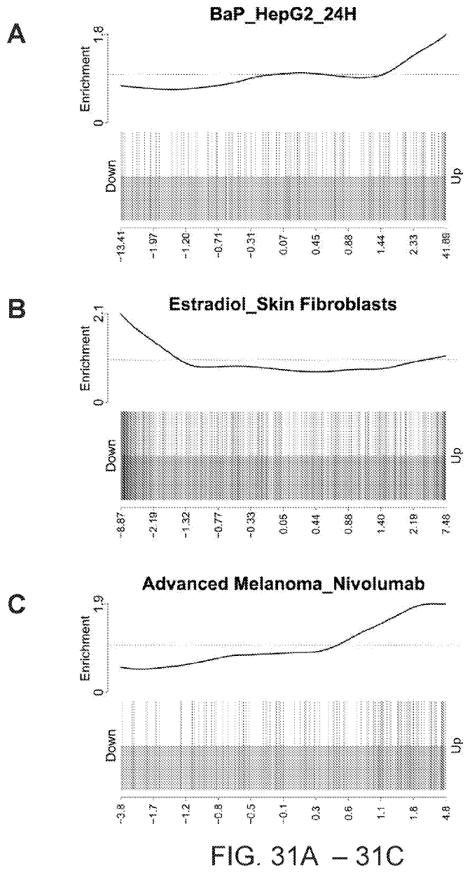

Using an AHR signature comprising of all the biomarkers in Table 1, allows the detection of AHR modulation caused by both direct and indirect AHR modulators in a cell type and ligand type independent fashion. This approach allowed us to detect the modulation of AHR in HepG2 cells treated with the environmental toxin BaP (FIG. 31A), in human skin fibroblast cells derived from hypospadias patients exposed to estradiol that modulates the activity of the estrogen receptor, which is a known binding partner of AHR (FIG. 31B) and in tumor tissue of advanced melanoma patients after receiving immune checkpoint inhibition by Nivolumab (FIG. 31C), which is an example of an indirect modulation of AHR through immunotherapy.

### Example 6: AHR Activation Signatures for 32 Different Cancer Types

Using the methodology described herein, the inventors have defined AHR activation signature for 32 different cancer types. The cancers were selected from The Cancer Genome Atlas (TCGA) Program of The National Cancer Institute. The TCGA cancers and the AHR activation signatures are listed in Table 2.

TABLE 2
AHR Activation Signatures for 32 Different Cancer Types

| Cancer Type | AHR Activation Signature |
|---|---|
| TCGA_ACC | PTGS2; CD3E; CYBB; THBS1; TXNRD1; CAV1; NSDHL; NEDD9; NCOR2; F3 |
| TCGA_BLCA | THBS1; SERPINE1; PRDM1; HIF1A; PTGS2; TIPARP; FOS; CDKN1A; |
| | TFF1; AREG; MMP1; KIT; NFE2L2; LYN; NDRG1; EREG; MYC; AQP3; |
| | UGT1A6; SLC7A5; CD3E; NR1H4; ADM; EGR1; CAV1; NEDD9; |
| | GHR; CDK4; IRF8; ID1 |
| TCGA_BRCA | PLA2G4A; PRDM1; SERPINE1; CAV1; NR3C1; CYP1B1; PNPLA7; |
| | TGFBI; PDE2A; RSPO3; HIF1A; IL6; SLC10A1; MMP1; THBS1; CDKN1A; |
| | EDN1; CCL5; EGR1; CYBB; NFE2L2; CD3E; AHR; CDK4; NRIP1; |
| | LEPR; ADM |
| TCGA_CESC | PRDM1; SERPINE1; TIPARP; THBS1; ID1; FPR2; SOCS2; CDKN1A; LYN; |
| | PTGS2; GHR; DKK3; FAT1; TGFBI; FOS; ADM; GNA13; MYC; |
| | TGM1; CYBB; SESN2; HIF1A; OVOL1; UGT1A6 |
| TCGA_CHOL | NOS3; AHR; HMOX1; EREG; LHCGR; LIFR; NR3C1; CYP19A1; FOXQ1; |
| | ESR1; TGFBI; PLA2G4A; EGR1; PNPLA7; IL1B; TIPARP; KIT; CFTR; |
| | NR1H4; CCL5; CYP3A4; TH; INSIG1; NEDD9; PAX5 |
| TCGA_COAD | CYBB; CDKN1A; CAV1; SESN2; PRDM1; FOS; CD36; SMAD7; IL1B; |
| | CYP1A1; NEDD9; CD3E; NDRG1; DUOX2; JAG1; KIT; DKK3; CD8A; |
| | XDH; GATA3; PDE2A; EGR1; IRF8; TFF1; SLC10A1; JUP; SERPINB2; |
| | SCARB1; LTBP1; CYP1B1; ABCG2; CYP2B6; PNPLA7; CYP19A1; SLC7A5; |
| | TGM1; TGFBI; LHCGR; MMP1; NR1H4; SH3KBP1 |
| TCGA_DLBC | FPR2; TXNRD1; CCND1; ATP6AP2; NDRG1; DKK3; FBXO32; TJP1; |
| | NANOG |
| TCGA_ESCA | SLC10A1; HIF1A; NANOG; CDKN1A; PRDM1; JAG1; THBS1; ATP6AP2; |
| | PIW1L2; CYP1A2; HMOX1; TIPARP; SPRR2D; CYP19A1; FAT1; |
| | NOS3; SERPINB2; CYBB; F3; IL6; EDN1 |
| TCGA_GBM | PRDM1; CYBB; LYN; TIPARP; FBXO32; SCIN; SERPINE1; PTGS2; |
| | SLC7A5; CYP1B1; NEDD9; IL6; NFE2L2; JUP; IL1R2; CCL5; LTBP1; |
| | IGFBP1; HMOX1; IKZF3 |
| TCGA_HNSC | NRIP1; PRDM1; THBS1; TIPARP; FPR2; KIT; CAV1; EPGN; CYBB; |
| | PNPLA7; PDE2A; LEPR; TGM1; REL; DKK3; FAT1; MMP1; JUP; ABCG2; |
| | SLC7A5; SLC10A1; AQP3; NR3C1; TJP1; CDKN1A; NANOG; KDM1A; |
| | NEDD9; FLG |
| TCGA_KICH | THBS1; CYBB; PRDM1; UGT1A6; EDN1; LEPR; NEDD9; SORL1; |
| | NFE2L2; EGR1; AREG; PTGS2; AHR; DUOX2; EGFR; LHCGR; HIF1A; |
| | ABCC4; IGFBP1 |
| TCGA_KIRC | PNPLA7; NANOG; CYP2E1; GNA13; NR3C1; THBS1; PRDM1; SLC10A1; |
| | CYP1B1; NEDD9; INSIG1; SERPINE1; PDE2A; PTGS2; IL6; NOS3; |
| | CDKN1A; TXNRD1; CYP1A2; ABCG2; EGFR; FOS; CAV1 |
| TCGA_KIRP | PRDM1; PLA2G4A; LYN; TGM1; CYP2E1; UGT1A6; SERPINE1; |
| | NOS3; GNA13; THBS1; PNPLA7; HIF1A |
| TCGA_LGG | CYP1B1; LYN; TGM1; PRDM1; PNPLA7; GNA13; THBS1; SORL1; PTGS2; |
| | CCL5; LEPR; AREG; DKK3; HMOX1; CAV1; FOS; ABCG2; |
| | ACTA2; CDKN1A; LTBP1; FAT1; AMIGO2; TJP1; ARG2; NEDD9 |
| TCGA_LIHC | PRDM1; PTGS2; NOS3; PDE2A; RSPO3; CYP1A2; INS1G1; IL6; HSD17B4; |
| | CYBB; LPL; SERPINE1; KMO; FPR2; TRF8; FOS; AHR; HIF1A; |
| | DKK3; LIFR; THBS1; GHR; TGM1; CYP3A4; SOCS2; KDM1A |
| TCGA_LUAD | FPR2; CDKN1A; CAV1; SERPINE1; PRDM1; SLC10A1; THBS1; IL1R2; |
| | ABCG2; HMOX1; EGR1; SH3KBP1; DKK3; IRF8; IL6; FOS; CYP2E1; |
| | LEPR; HIF1A; IGFBP1; CD36; CD3E; CYP1B1; FAT1; HSPB2; LPL |
| TCGA_LUSC | THBS1; LYN; SERPINE1; CYBB; PNPLA7; CDKN1A; ADM; PRDM1; |
| | SPRR2D; CAV1; EPGN; LPL; SLC7A5; DKK3; SERPINB2; KDM1A; |
| | ABCG2; NDRG1; UGT1A6; FOS; CYP1B1; HIF1A; F3; CD36; AMIGO2; |
| | TIPARP; PLA2G4A; RFC3; HMOX1 |
| TCGA_MESO | HIF1A; THBS1; PRDM1; AREG; TIPARP; HMOX1; F3; NEDD9; NRIP1; |
| | CD8A; EREG; INSIG1; GFI1 |
| TCGA_OV | TGFBI; LYN; CYBB; IRF8; SERPINE1; FAS; IL6; FPR2; EGFR; HMOX1; |
| | PTGS2; F3; TIPARP; EDN1; ATP6AP2; ACTA2; FBXO32; NDRG1; |
| | NEDD9; CD8A; ID2; NR3C1; GHR; TNFSF9; BLNK; SLC7A5; LTBP1; |
| | EGR1; HIF1A; DKK3 |
| TCGA_PAAD | THBS1; GNA13; CYBB; HMOX1; GHR; IRF8; BCL2; FAT1; DKK3; |
| | NRIP1; SERPINB2; PNPLA7; EREG; F3; NCOR2; IGT1A6; SORL1 |
| TCGA_PCPG | THBS1; MYC; LTBP1; SERPINE1; IL1R2; PRDM1; PTGS2; AREG; IL6; |
| | CD36; JAG1; TIPARP; NOS3; IL1B; EGR1; NFE2L2; IKZF3; CYBB; |
| | PCK1; TNFSF9 |
| TCGA_PRAD | NR3C1; THBS1; TIPARP; CFTR; LTBP1; LEPR; CAV1; PTGS2; CXCL2; |
| | CDKN1A; PNPLA7; EGR1; REL; AREG; IL6; FBXO32; ATP6AP2; |
| | EREG; CYBB; GNA13; ID2; HIF1A; CD8A; MID1 |
| TCGA_READ | PRDM1; CDKN1A; CRH; GNA13; SERPINE1; CYBB; DKK3; CD36; |
| | GHR |
| TCGA_SARC | CYBB; IL6; FPR2; PLA2G4A; FOS; SERPINB2; AHR; AQP3; PRDM1; |
| | CD3E; CDK4; NOS3; HIF1A; KDM1A; SCIN; AREG; CD8A; DKK3; |
| | CCL5; KIT; PNPLA7; ADM; HSD17B4 |
| TCGA_SKCM | AQP3; PRDM1; OVOL1; HMOX1; IGF2; FPR2; SERPINB2; STC2; FGFR2; |
| | AREG; EGFR; IL6; ACTA2; DLX3; ID2 |
| TCGA_STAD | NANOG; CDKN1A; THBS1; CVP1A2; SLC10A1; FPR2; CYBB; PRDM1; |
| | HMOX1; KIT; Z1C3; FAT1; IRF8; UGT1A6; NDRG1; FOS; JAG1; VAV3; |
| | SMAD7; NEDD9; PDE2A; CD36; TFF1; EGR1; HSD17B4 |
| TCGA_TGCT | PLA2G4A; CDKN1A; SCARB1; TGFB1; TFF1; THBS1; IRF8; JAG1; |
| | LYN; CFTR; SMAD7; FAS; AHR; CD36 |
| TCGA_THCA | AHR; NEDD9; MYC; IL6; PTGS2; CYP2E1; NRIP1; SERPINE1; FPR2; |
| | CYBB; EREG; SERPINB2; GHR; LEPR; AREG; PIWIL2 |
| TCGA_THYM | PRDM1; THBS1; FAS; NOS3; GHR; F3; NQO1; SMAD3; ADM; CDKN1A; |
| | CAV1; IL6; NDRG1; CYP19A1; ABCC4; IL1B; MID1; FBXO32; |
| | FOXQ1; CCND1; FLG; CYBB; EPGN; TGM1; CDK4; SERPINB2; TFF1 |
| TCGA_UCEC | PRDM1; THBS1; AHR; CYBB; PTGS2; FOS; JUP; FAS; SERPINE1; |
| | CYP1A2; FAT1; CAV1; HIF1A; SLC10A1; SLC7A5; LYN; IRF8; |
| | EGR1; AREG; EGFR; NANOG; FOXQ1; AQP3; NDRG1; |
| | CDKN1A; HMOX1; FRXO32; JAG1; SERPINB2 |
| TCGA_UCS | PRDM1; EDN1; AREG; NEDD9; FBXO32; AMIGO2; NDRG1; LYN; |
| | EGFR; THBS1; CYBB; BLNK; CAV1; IRF8; STC2; IL6; AQP3 |
| TCGA_UVM | AQP3; PDE2A; PTGS2; CDKN1A; AMIGO2; CD3E; NOS3; DKK3; IL6; |
| | SMAD3; ESR1; CD8A; ACTA2; PHGDH; CCND1 |

### Example 7: AHR Activation Subsignatures for 32 Cancer Types

Inventors further classified the AHR activation signatures of Table 2 using Kmeans clustering, and determined different subsignatures within the AH-R activation signature as shown in Table 3.

TABLE 3
Tabular representation of the different AHR signature biomarkers for the 32 TCGA cancers divided
among the different AHR subgroups for each cancer entity defined by consensus Kmeans clustering.

| Cancer Type | Subgroup 1 | Subgroup 2 | Subgroup 3 | Subgroup 4 |
|---|---|---|---|---|
| TCGA_ACC | NSDHL | PTGS2; CAV1; NEDD9; | — | — |
| | | F3; THBS1; CYBB; | | |
| | | NCOR2; TXNRD1; CD3E | | |
| TCGA_BLCA | PRDM1; HIF1A; | ID1; TFF1 | NFE2L2; KIT; | PTGS2; NEDD9; |
| | SLC7A5; | | AQP3; UGT1A6 | GHR; EGR1; |
| | NDRG1; CAV1; | | | THBS1; IRF8; |
| | SERPINE1; | | | FOS; CD3E |
| | AREG; CDKN1A; | | | |
| | EREG; CDK4; | | | |
| | MYC; ADM; | | | |
| | TIPARP; MMP1; | | | |
| | LYN | | | |
| TCGA_BRCA | PRDM1; EDN1; | HIF1A; PLA2G4A; | PNPLA7 | NRIP1 |
| | CAV1; SERPINE1; | CDK4; ADM; | | |
| | AHR; NR3C1; NFE2L2; | MMP1; CD3E; | | |
| | LEPR; TGFBI; | CCL5 | | |
| | EGR1; CDKN1A; | | | |
| | IL6; THBS1; | | | |
| | CYP1B1; RSPO3; | | | |
| | CYBB; PDE2A | | | |
| TCGA_CESC | TGM1; TGFBI; | | DKK3; PRDM1; | PTGS2 |
| | ID1; MYC; | | FAT1; HIF1A; | |
| | ADM; OVOL1 | | SERPINE1; GHR; | |
| | | | GNA13; SOCS2; | |
| | | | CDKN1A; SESN2; | |
| | | | THBS1; TIPARP; | |
| | | | CYBB; UGT1A6; | |
| | | | FOS; LYN | |
| TCGA_CHOL | ESR1; HMOX1; | CFTR; AHR; | | NR1H4; KIT; |
| | LIFR; IL1B; | NEDD9; NR3C1; | | INSIG1 |
| | PNPLA7; CYP3A4; | PLA2G4A; TGFBI; | | |
| | CCL5 | EGR1; EREG; | | |
| | | TIPARP; FOXQ1; | | |
| | | NOS3 | | |
| TCGA_COAD | LTBP1; PRDM1; | SCARB1; SLC7A5; | DKK3; JAG1; | NDRG1; CDKN1A; |
| | SMAD7; | TGFBI; JUP; CYP2B6 | CAV1; GATA3; | IL1B; DUOX2; |
| | PNPLA7 | | NEDD9; ABCG2; | XDH; TFF1; |
| | | | EGR1; SESN2; | FOS |
| | | | CD36; CYP1B1; | |
| | | | IRF8; SH3KBP1; | |
| | | | CD8A; KIT; CYBB; | |
| | | | PDE2A; MMP1; | |
| | | | CD3E | |
| TCGA_DLBC | | DKK3; TJP1; NDRG1; | — | — |
| | | CCND1; FBXO32; | | |
| | | FPR2; ATP6AP2; | | |
| | | TXNRD1 | | |
| TCGA_ESCA | ATP6AP2 | PRDM1; HIF1A; | FAT1 | EDN1; HMOX1; |
| | | JAG1; F3; CDKN1A; | | IL6; THBS1; |
| | | SPRR2D; TIPARP; | | NOS3; CYBB; |
| | | SERPINB2 | | PIWIL2 |
| TCGA_GBM | SLC7A5 | | SCIN; LTBP1; | JUP |
| | | | PRDM1; PTGS2; | |
| | | | HMOX1; SERPINE1; | |
| | | | NEDD9; IL1R2; | |
| | | | NFE2L2; IL6; | |
| | | | CYP1B1; IGFBP1; | |
| | | | IBXO32; IKZF3; | |
| | | | TIPARP; CYBB; | |
| | | | LYN; CCL5 | |
| TCGA_HNSC | DKK3; PRDM1; | KDM1A; TGM1; | | NEDD9; PNPLA7; |
| | FAT1; TJP1; | SLC7A5; CAV1; | | KIT |
| | NR3C1; LEPR; | CDKN1A; FLG; | | |
| | ABCG2; THBS1; | TIPARP; AQP3; | | |
| | REL; CYBB; | JUP; EPGN | | |
| | FPR2; NRIP1; | | | |
| | PDE2A; MMP1 | | | |
| TCGA_KICH | | PRDM1; PTGS2; | — | — |
| | | EDN1; HIF1A; | | |
| | | AHR; AREG; NEDD9; | | |
| | | NFE2L2; | | |
| | | LEPR; EGR1; ABCC4; | | |
| | | SORL1; THBS1; | | |
| | | EGFR; CYBB; | | |
| | | UGT1A6 | | |
| TCGA_KIRC | PRDM1; PTGS2; | INSIG1; | SERPINE1; CYP2E1; | — |
| | CAV1; | TXNRD1 | PNPLA7; IL6 | |
| | NEDD9; NR3C1; | | | |
| | ABCG2; GNA13; | | | |
| | CDKN1A; | | | |
| | THBS1; CYP1B1; | | | |
| | EGFR; NOS3; | | | |
| | FOS; PDE2A | | | |
| TCGA_KIRP | | PRDM1; HIF1A; | TGM1; CYP2E1; | — |
| | | SERPINE1; | PNPLA7 | |
| | | PLA2G4A; GNA13; | | |
| | | THBS1; NOS3; | | |
| | | UGT1A6; LYN | | |
| TCGA_LGG | LTBP1; PRDM1; | ABCG2; CDKN1A | DKK3; PTGS2; | TGM1; |
| | HMOX1; CAV1; | | ARG2; FAT1; | PNPLA7 |
| | ACTA2; | | TJP1; LEPR; | |
| | NEDD9; GNA13; | | SORL1 | |
| | THBS1; | | | |
| | CYP1B1; AMIGO2; | | | |
| | FOS; LYN; | | | |
| | CCL5 | | | |
| TCGA_LIHC | DKK3; PRDM1; | | KDM1A; HSD17B4 | — |
| | PTGS2; HIF1A; | | | |
| | SERPINE1; | | | |
| | AHR; GHR; | | | |
| | LIFR; KMO; | | | |
| | SOCS2; THBS1; | | | |
| | CYP1A2; IRF8; | | | |
| | CYP3A4; | | | |
| | NOS3; CYBB; | | | |
| | FOS; LPL; | | | |
| | INSIG1; PDE2A | | | |
| TCGA_LUAD | DKK3; PRDM1; | HMOX1; IL1R2; | CYP2E1; FOS; | |
| | FAT1; HIF1A; | IL6; IRF8; CD3E | LPL | |
| | CAV1; | | | |
| | SERPINE1; LEPR; | | | |
| | ABCG2; EGR1; | | | |
| | CDKN1A; | | | |
| | CD36; THBS1; | | | |
| | CYP1B1; SH3KBP1; | | | |
| | FPR2 | | | |
| TCGA_LUSC | KDM1A; RFC3 | SLC7A5; NDRG1; | DKK3; PRDM1; | HIF1A; CAV1; |
| | | ADM; SPRR2D; | HMOX1; F3; | SERPINE1; |
| | | UGT1A6 | ABCG2; PNPLA7; | PLA2G4A; |
| | | | CD36; THBS1; | CDKN1A; AMIGO2; |
| | | | CYP1B1; CYBB; | TIPARP; |
| | | | FOS; LPL; LYN | EPGN; |
| | | | | SERPINB2 |
| TCGA_MESO | | PRDM1; HMOX1; | — | — |
| | | HIF1A; AREG; | | |
| | | NEDD9; F3; THBS1; | | |
| | | CD8A; GFI1; | | |
| | | TIPARP; NRIP1; | | |
| | | INSIG1 | | |
| TCGA_OV | FAS; LTBP1; | | | |
| | DKK3; PTGS2; | | | |
| | EDN1; BLNK; | | | |
| | HMOX1; HIF1A; | | | |
| | SLC7A5; | | | |
| | NDRG1; SERPINE1; | | | |
| | ACTA2; | | | |
| | NEDD9; GHR; | | | |
| | NR3C1; ID2; | | | |
| | F3; TGFBI; EGR1; | | | |
| | TNFSF9; IL6; | | | |
| | IRF8; EGFR; | | | |
| | CD8A; FBXO32; | | | |
| | TIPARP; | | | |
| | CYBB; ATP6AP2; | | | |
| | LYN | | | |
| TCGA_PAAD | PNPLA7; | DKK3; FAT1; | — | — |
| | NCOR2 | HMOX1; GHR; F3; | | |
| | | GNA13; EREG; | | |
| | | SORL1; THBS1; | | |
| | | IRF8; CYBB; BCL2; | | |
| | | NRIP1; SERPINB2 | | |
| TCGA_PCPG | | LTBP1; PRDM1; | — | — |
| | | PTGS2; JAG1; | | |
| | | SERPINE1; AREG; | | |
| | | NFE2L2; EGR1; | | |
| | | IL1B; CD36; | | |
| | | IL6; MYC; THBS1; | | |
| | | IKZF3; TIPARP; | | |
| | | NOS3; CYBB | | |
| TCGA_PRAD | CFTR; LTBP1; | PNPLA7 | — | — |
| | PTGS2; CXCL2; | | | |
| | HIF1A; MID1; | | | |
| | CAV1; AREG; | | | |
| | NR3C1; ID2; | | | |
| | LEPR; GNA13; | | | |
| | EGR1; CDK | | | |
| | N1A; IL6; THB | | | |
| | S1; CD8A; | | | |
| | FBXO32; REL; | | | |
| | TIPARP; CYBB; | | | |
| | ATP6AP2 | | | |
| TCGA_READ | | DKK3; PRDM1; | | CDKN1A |
| | | SERPINE1; GHR; | | |
| | | GNA13; CD36; | | |
| | | CYBB | | |
| TCGA_SARC | PNPLA7; | SCIN; PRDM1; | KDM1A; FOS | DKK3; HIF1A; |
| | HSD17B4 | AREG; CDK4; IL6; | | AHR; PLA2G4A |
| | | ADM; CD8A; KIT; | | |
| | | NOS3; CYBB; | | |
| | | AQP3; FPR2; | | |
| | | CD3E; CCL5 | | |
| TCGA_SKCM | | PRDM1; DLX3; | — | — |
| | | FGFR2; HMOX1; | | |
| | | ACTA2; STC2; ID2; | | |
| | | IL6; EGFR; | | |
| | | AQP3; IGF2; OVOL1; | | |
| | | SERPINB2 | | |
| TCGA_STAD | PRDM1; FAT1; | IFF1; UGT1A6 | — | — |
| | HMOX1; JAG1; | | | |
| | SMAD7; | | | |
| | NDRG1; NEDD9; | | | |
| | EGR1; CDKN1A; | | | |
| | HSD17B4; | | | |
| | VAV3; CD36; | | | |
| | THBS1; IRF8; | | | |
| | KIT; CYBB; | | | |
| | FOS; FPR2; PDE2A | | | |
| TCGA_TGCT | SMAD7; LYN | 1RF8 | CFTR; FAS; SCARB1; | — |
| | | | JAG1; AHR; | |
| | | | PLA2G4A; TGFBI; | |
| | | | CDKN1A; CD36; | |
| | | | THBS1; TFF1 | |
| TCGA_THCA | PTGS2; SERPINE1; | GHR; PIWIL2 | CYP2E1 | — |
| | AHR; AREG; | | | |
| | NEDD9; LEPR; | | | |
| | EREG; IL6; | | | |
| | MYC; CYBB; | | | |
| | NRIP1 | | | |
| TCGA_THYM | | CDK4 | FAS; PRDM1; TGM1; | — |
| | | | MID1; NDRG1; | |
| | | | CAV1; CCND1; | |
| | | | GHR; F3; | |
| | | | CDKN1A; ABCC4; | |
| | | | IL1B; THBS1; | |
| | | | ADM; FBXO32; | |
| | | | FOXQ1; NOS3; | |
| | | | CYBB; SMAD3; | |
| | | | NQO1 | |
| TCGA_UCEC | FAS; PRDM1; | | NDRG1; JUP | — |
| | PTGS2; FAT1; | | | |
| | HMOX1; HIF1A; | | | |
| | JAG1; SLC7A5; | | | |
| | CAV1; SERPINE1; | | | |
| | AHR; AREG; | | | |
| | EGR1; CDKN1A; | | | |
| | THBS1; | | | |
| | IRF8; | | | |
| | EGFR; FBXO32; | | | |
| | FOXQ1; CYBB; | | | |
| | AQP3; FOS; | | | |
| | LYN | | | |
| TCGA_UCS | | PRDM1; EDN1; | — | — |
| | | BLNK; NDRG1; | | |
| | | CAV1; AREG; NEDD9; | | |
| | | STC2; IL6; | | |
| | | THBS1; AMIGO2; | | |
| | | IRF8; EGFR; | | |
| | | FBXO32; CYBB; | | |
| | | AQP3; LYN | | |
| TCGA_UVM | | PHGDH; ACTA2; | DKK3; ESR1; CCND1; | — |
| | | NOS3; SMAD3; | CDKN1A; | |
| | | PDE2A | AMIGO2; CD8A; | |
| | | | AQP3; CD3E | |

### Example 8: AHR Activation Subsignatures for 32 Cancer Types 2

Inventors further classified the AH-R activation signature of Table 2 using non-negative matrix factorization (NMF) clustering to determine different subsignatures within the AHR activation signature as shown in Table 4. Interestingly, different clustering methodologies gave very similar results, validating the strength of AHR biomarkers and AH-R activation signatures.

TABLE 4
Tabular representation of the different AHR signature biomarkers for the 32 TCGA cancers divided
among the different AHR subgroups for each cancer entity defined by consensus NMF clustering

| Tumor Type | Subgroup 1 | Subgroup 2 | Subgroup 3 | Subgroup 4 | Subgroup 5 | Subgroup 6 |
|---|---|---|---|---|---|---|
| TCGA_ACC | PTGS2; | NSDHL | — | — | — | — |
| | CAV1; | | | | | |
| | NEDD9; F3; | | | | | |
| | THBS1; | | | | | |
| | CYBB; | | | | | |
| | NCOR2; | | | | | |
| | TXNRD1; CD3E | | | | | |
| TCGA_BLCA | UGT1A6 | NEDD9; | PRDM1; | ID1; TFF1 | — | — |
| | | GHR; EGR1; | PTGS2; HIF1A; | | | |
| | | THBS1; | SLC7A5; | | | |
| | | IRF8; KIT; | NDRG1; CAV1; | | | |
| | | FOS; CD3E | SERPINE1; | | | |
| | | | AREG; | | | |
| | | | NFE2L2; | | | |
| | | | CDKN1A; EREG; | | | |
| | | | CDK4; | | | |
| | | | MYC; ADM; TIP | | | |
| | | | ARP; AQP3; | | | |
| | | | MMP1; LYN | | | |
| TCGA_BRCA | | PRDM1; | | AHR; NFE2L2; | EDN1; | — |
| | | HIF1A; PLA2G4A; | | THBS1; CYP1B1; | CAV1; | |
| | | TGFBI; | | NRIP1 | SERPINE1; | |
| | | CDK4; ADM; | | | NR3C1; | |
| | | CYBB; MMP1; | | | LEPR; | |
| | | CD3E; CCL5 | | | EGR1; | |
| | | | | | CDKN1A; | |
| | | | | | PNPLA7; | |
| | | | | | IL6; | |
| | | | | | RSPO3; | |
| | | | | | PDE2A | |
| TCGA_CESC | | OVOL1 | DKK3; PTGS2; | | PRDM1; | — |
| | | | TGM1; HIF1A; | | FAT1; | |
| | | | SERPINE1; | | GHR; | |
| | | | TGFBI; | | GNA13; | |
| | | | SOCS2; | | SESN2; | |
| | | | CDKN1A; | | CYBB; | |
| | | | ID1; MYC; | | UGT1A6; | |
| | | | THBS1; ADM; | | LYN | |
| | | | TIPARP; | | | |
| | | | FOS | | | |
| TCGA_CHOL | CFTR; | ESR1; | PNPLA7 | NR1H4; | — | — |
| | AHR; NEDD9; | HMOX1; NR3C1; | | INSIG1 | | |
| | PLA2G4A; | LIFR; | | | | |
| | TGFBI; | EGR1; CYP3A4; | | | | |
| | EREG; IL1B; | TIPARP; | | | | |
| | KIT; CCL5 | FOXQ1; | | | | |
| | | NOS3 | | | | |
| TCGA_COAD | IL1B; | SLC7A5; | LTBP1; | | SCARE1; | — |
| | IRF8; XDH | TGFBI; | DKK3; PRDM1; | | TGM1; | |
| | | CYP2B6 | JAG1; SMAD7; | | NDRG1; | |
| | | | CAV1; | | CDKN1A; | |
| | | | GATA3; | | PNPLA7; | |
| | | | NEDD9; ABCG2; | | SESN2; | |
| | | | EGR1; CD36; | | DUOX2; | |
| | | | CYP1B1; | | TFF1; | |
| | | | SH3KBP1; | | FOS; JUP | |
| | | | CD8A; KIT; | | | |
| | | | CYBB; PDE2A; | | | |
| | | | MMP1; | | | |
| | | | CD3E | | | |
| TCGA_DLBC | NDRG1; | | DKK3; TJP1; | | — | — |
| | CCND1; | | FBXO32 | | | |
| | FPR2; | | | | | |
| | ATP6AP2; | | | | | |
| | TXNRD1 | | | | | |
| TCGA_ESCA | PRDM1; | EDN1; | FAT1; IL6; | ATP6AP2 | — | — |
| | HIF1A; | HMOX1; F3; | THBS1 | | | |
| | JAG1; | NOS3; CYBB; | | | | |
| | CDKN1A; | PIWIL2 | | | | |
| | SPRR2D; | | | | | |
| | TIPARP; | | | | | |
| | SERPINB2 | | | | | |
| TCGA_GBM | SLC7A5; | IGFBP1 | SCIN; LTBP1; | | — | — |
| | JUP | | PRDM1; | | | |
| | | | PTGS2; HMOX1; | | | |
| | | | SERPINE1; | | | |
| | | | NEDD9; | | | |
| | | | IL1R2; NFE2L2; | | | |
| | | | IL6; CYP1B1; | | | |
| | | | FBXO32; | | | |
| | | | IKZF3; | | | |
| | | | TIPARP; | | | |
| | | | CYBB; LYN; | | | |
| | | | CCL5 | | | |
| TCGA_HNSC | KDM1A; | DKK3; PRDM1; | | NEDD9; | — | — |
| | TGM1; | FAT1; | | PNPLA7 | | |
| | SLC7A5; | TJP1; NR3C1; | | | | |
| | CAV1; | LEPR; | | | | |
| | CDKN1A; | ABCG2; | | | | |
| | FLG; TIPARP; | THBS1; KIT; | | | | |
| | AQP3; | REL; CYBB; | | | | |
| | JUP; | FPR2; | | | | |
| | EPGN | NRIP1; PDE2A; | | | | |
| | | MMP1 | | | | |
| TCGA_KICH | HIF1A; | | IGFBP1 | PRDM1; | — | — |
| | NEDD9; | | | PTGS2; EDN1; | | |
| | NFE2L2; | | | AHR; | | |
| | LEPR; | | | AREG; | | |
| | ABCC4; | | | EGR1; THBS1; | | |
| | SORL1; | | | DUOX2; | | |
| | UGT1A6 | | | EGFR; CYBB | | |
| TCGA_KIRC | NEDD9; | TXNRD1 | PRDM1; | CYP2E1; | — | — |
| | NR3C1; | | PTGS2; CAV1; | PNPLA7 | | |
| | ABCG2; | | SERPINE1; | | | |
| | GNA13; | | CDKN1A; | | | |
| | EGFR | | IL6; THBS1; | | | |
| | | | CYP1B1; | | | |
| | | | NOS3; FOS; | | | |
| | | | INSTG1; PDE2A | | | |
| TCGA_KIRP | TGM1; | PRDM1; | | — | — | — |
| | CYP2E1; | HIF1A; | | | | |
| | PNPLA7 | SERPINE1; | | | | |
| | | PLA2G4A; | | | | |
| | | GNA13; THBS1; | | | | |
| | | NOS3; | | | | |
| | | UGT1A6; | | | | |
| | | LYN | | | | |
| TCGA_LGG | DKK3; PTGS2; | ABCG2; | LTBP1; PRDM1; | FATL1; | TGM1; | — |
| | ARG2; | CDKN1A | HMOX1; | GNA13 | PNPLA7 | |
| | TJP1; | | CAV1; | | | |
| | LEPR; | | ACTA2; | | | |
| | SORL1 | | NEDD9; THBS1; | | | |
| | | | CYP1B1; | | | |
| | | | AMIGO2; FOS; | | | |
| | | | LYN; | | | |
| | | | CCL5 | | | |
| TCGA_LIHC | AHR; | KDM1A; | DKK3; PRDM1; | | — | — |
| | HSD17B4; | HIF1A | PTGS2; | | | |
| | CYP1A2; | | SERPINE1; | | | |
| | CYP3A4 | | GHR; LIFR; | | | |
| | | | KMO; SOCS2; | | | |
| | | | THBS1; | | | |
| | | | IRF8; NOS3; | | | |
| | | | CYBB; FOS; | | | |
| | | | LPL; INSIG1; | | | |
| | | | PDE2A | | | |
| TCGA_LUAD | PRDM1; | | | DKK3; | — | — |
| | HMOX1; | | | FAT1; CAV1; | | |
| | HIF1A; | | | SERPINE1; | | |
| | IL1R2; IL6; | | | LEPR; | | |
| | CYPIB1; | | | ABCG2; | | |
| | IRF8; | | | EGR1; CDKN1A; | | |
| | SH3KBP1; | | | CYP2E1; | | |
| | FPR2; | | | CD36; | | |
| | CD3E | | | THBS1; | | |
| | | | | FOS; LPL | | |
| TCGA_LUSC | PLA2G4A; | SLC7A5; | KDM1A; | HIF1A; | DKK3; | — |
| | ABCG2; | ADM; SPRR2D; | PNPLA7 | NDRG1; | PRDM1; | |
| | RFC3; | UGT1A6; | | CAV1; SERPINE1; | HMOX1; | |
| | TIPARP; | SERPINB2 | | F3; CDKN1A; | CD36; THBS1; | |
| | LYN | | | AMIGO2; | CYP1B1; | |
| | | | | EPGN | CYBB; | |
| | | | | | FOS; LPL | |
| TCGA_MESO | PRDM1; | | | | — | — |
| | HMOX1; | | | | | |
| | HIF1A; | | | | | |
| | AREG; | | | | | |
| | NEDD9; F3; | | | | | |
| | THBS1; | | | | | |
| | CD8A; GFI1; | | | | | |
| | TIPARP; | | | | | |
| | NRIP1; | | | | | |
| | INSIG1 | | | | | |
| TCGA_OV | FAS; LTBP1; | | | — | — | — |
| | DKK3; | | | | | |
| | PTGS2; | | | | | |
| | EDN1; | | | | | |
| | BLNK; HMOX1; | | | | | |
| | HIF1A; | | | | | |
| | SLC7A5; | | | | | |
| | NDRG1; | | | | | |
| | SERPINE1; | | | | | |
| | ACTA2; | | | | | |
| | NEDD9; GHR; | | | | | |
| | NR3C1; TD2; F3; | | | | | |
| | TGFB1; EGR1; | | | | | |
| | TNFSF9; IL6; | | | | | |
| | IRF8; EGFR; | | | | | |
| | CD8A; | | | | | |
| | FBXO32; | | | | | |
| | TIPARP; | | | | | |
| | CYBB; ATP6 | | | | | |
| | AP2; LYN | | | | | |
| TCGA_PAAD | HMOX1; | F3 | SERPINB2 | | PNPLA7; | DKK3; |
| | GHR; | | | | NCOR2 | FAT1; |
| | SORL1; | | | | | GNA13; EREG; |
| | THBS1; IRF8; | | | | | UGT1A6 |
| | CYBB; | | | | | |
| | BCL2; | | | | | |
| | NRIP1 | | | | | |
| TCGA_PCPG | | | LTBP1; | — | — | — |
| | | | PRDM1; PTGS2; | | | |
| | | | JAG1; SERPINE1; | | | |
| | | | AREG; NFE2L2; | | | |
| | | | EGR1; IL1B; | | | |
| | | | CD36; IL6; | | | |
| | | | MYC; THBS1; | | | |
| | | | IKZF3; | | | |
| | | | TIPARP; | | | |
| | | | NOS3; CYBB | | | |
| TCGA_PRAD | CD8A; CYBB; ATP6AP2 | | PNPLA7 | CXCL2; | CFTR; | — |
| | | | | CAV1; AREG; | LTBP1; | |
| | | | | CDKN1A; | PTGS2; | |
| | | | | IL6; | HIF1A; | |
| | | | | FBXO32 | MID1; NR3C1; | |
| | | | | | ID2; LEPR; | |
| | | | | | GNA13; | |
| | | | | | EGR1; | |
| | | | | | THBS1; REL; | |
| | | | | | TIPARP | |
| TCGA_READ | CDKN1A | DKK3; | | — | — | — |
| | | PRDM1; | | | | |
| | | SERPINE1; | | | | |
| | | GHR; GNA13; | | | | |
| | | CD36; | | | | |
| | | CYBB | | | | |
| TCGA_SARC | SC1N; | PNPLA7; | KIT; FOS | KDM1A; | — | — |
| | PRDM1; CDK4; | HSD17B4 | | DKK3; | | |
| | IL6; ADM; | | | HIF1A; AHR; | | |
| | CD8A; | | | PLA2G4A | | |
| | NOS3; | | | | | |
| | CYBB; | | | | | |
| | AQP3; FPR2; | | | | | |
| | CD3E; | | | | | |
| | CCL5 | | | | | |
| TCGA_SKCM | ID2 | PRDM1; | ACTA2 | | — | — |
| | | DLX3; FGFR2; | | | | |
| | | HMOX1; | | | | |
| | | AREG; STC2; | | | | |
| | | IL6; EGFR; | | | | |
| | | AQP3; EGF2; | | | | |
| | | OVOL1; | | | | |
| | | SERPINB2 | | | | |
| TCGA_STAD | PRDM1; | NDRG1; | SMAD7; | HMOX1; | NEDD9; EGR1; CD36; | |
| | FAT1; | TFF1; UGT1A6; | VAV3 | CDKN1A; | THBS1; KIT; | |
| | JAG1; HSD17B4; | FOS | | IRF8 | PDE2A | |
| | CYBB; | | | | | |
| | FPR2 | | | | | |
| TCGA_TGCT | | CFTR; FAS; | SMAD7 | IRF8; LYN | — | — |
| | | SCARB1; | | | | |
| | | JAG1; AHR; | | | | |
| | | PLA2G4A; | | | | |
| | | TGFBI; CDKN1A; | | | | |
| | | CD36; | | | | |
| | | THBS1; | | | | |
| | | TFF1 | | | | |
| TCGA_THCA | GHR; | | CYP2E1 | PTGS2; | EREG; | — |
| | PIWIL2 | | | SERPINE1; | IL6; | |
| | | | | AHR; | CYBB | |
| | | | | AREG; NEDD9; | | |
| | | | | LEPR; | | |
| | | | | MYC; | | |
| | | | | NRIP1 | | |
| TCGA_THYM | FAS; PRDM1; | | CDK4 | — | — | — |
| | TGM1; MID1; | | | | | |
| | NDRG1; | | | | | |
| | CAV1; | | | | | |
| | CCND1; | | | | | |
| | GHR; F3; | | | | | |
| | CDKN1A; | | | | | |
| | ABCC4; | | | | | |
| | IL1B; THBS1; | | | | | |
| | ADM; FBXO32; | | | | | |
| | FOXQ1; | | | | | |
| | NOS3; | | | | | |
| | CYBB; | | | | | |
| | SMAD3; | | | | | |
| | NQO1 | | | | | |
| TCGA_UCEC | PRDM1; | | FAS; PTGS2; | — | — | — |
| | FAT1; | | HIF1A; | | | |
| | HMOX1; | | NDRG1; AHR; | | | |
| | JAG1; | | EGFR; LYN | | | |
| | SLC7A5; | | | | | |
| | CAV1; | | | | | |
| | SERPINE1; | | | | | |
| | AREG; | | | | | |
| | EGR1; | | | | | |
| | CDKN1A; | | | | | |
| | THBS1; IRF8; | | | | | |
| | FBXO32; | | | | | |
| | FOXQ1; | | | | | |
| | CYBB; | | | | | |
| | AQP3; | | | | | |
| | FOS; JUP | | | | | |
| TCGA_UCS | | PRDM1; | — | — | — | — |
| | | EDN1; BLNK; | | | | |
| | | NDRG1; | | | | |
| | | CAV1; | | | | |
| | | AREG; NEDD9; | | | | |
| | | STC2; IL6; | | | | |
| | | THBS1; | | | | |
| | | AMIGO2; | | | | |
| | | IRF8; EGFR; | | | | |
| | | FBXO32; | | | | |
| | | CYBB; | | | | |
| | | AQP3; LYN | | | | |
| TCGA_UVM | PHGDH; | DKK3; ESR1; | | — | — | — |
| | ACTA2; | CCND1; | | | | |
| | NOS3; | CDKN1A; | | | | |
| | SMAD3; | AMIGO2; | | | | |
| | PDE2A | CD8A; | | | | |
| | | AQP3; CD3E | | | | |

### Example 9: Alternative/Secondary AHR Activation Signatures for 32 Cancer Types

The inventors have determined alternative (secondary) AH-R activation signatures based on proteomics (Reverse Phase Protein Array (RPPA)) data using Kmeans clustering as shown in Table 5. These alternative AHR activation signatures can be used to determine the AHR activation status of a sample.

TABLE 5
Tabular representation of the different RPPA features that could be used to call AHR activation for the 32 TCGA
cancers divided among the different AHR subgroups for each cancer entity defined by consensus Kmeans clustering.

| Tumor | | | | |
|---|---|---|---|---|
| Type | Group 1 | Group 2 | Group 3 | Group 4 |
| TCGA_ACC | X53BP1; | INPP4B; | — | — |
| | ACC_pS79; | NFKBP65_pS536; | | |
| | ACC1; | PAI1; | | |
| | AKT; | PKCALPHA; | | |
| | AKT_pS473; | PKCALPHA_pS657; | | |
| | AMPKALPHA_pT172; | STAT5ALPHA; | | |
| | ATM; CKIT; | G6PD; | | |
| | CYCLINE1; EEF2K; | PKCPANBETAII_pS660; | | |
| | GSK3ALPHABETA_pS21S9; | ACETYLATUBULINLYS40; | | |
| | MEK1; BRAF; | ANNEXIN1; | | |
| | GAPDH; | PREX1; SMAC | | |
| | NDRG1_pT346; | | | |
| | RAPTOR; BRD4 | | | |
| TCGA_BLCA | AMPKALPHA_pT172; | X4EBP1_pT37T46; | ACC_pS79; | ATM; CAVEOLIN1; |
| | CASPASE7CLEAVEDD198; | BETACATENIN; | ACC1; EGFR_pY1068; | FIBRONECTIN; |
| | CYCLINB1; EGFR; | CKIT; CLAUDIN7; | HER2_pY1248; | YAP_pS127; |
| | HSP70; PAI1; | ECADHERIN; | SRC_pY527; MSH6 | MYH11; RICTOR; |
| | G6PD; NDRG1_pT346; | GATA3; HER2; | | P16INK4A |
| | TRANSGLUTAMINASE; | HER3; INPP4B; | | |
| | P62LCKLIGAND; | P38_pT180Y182; | | |
| | ANNEXIN1 | RB_pS807S811; | | |
| | | VEGFR2; BRAF; | | |
| | | FASN; | | |
| | | RAB25; TFRC; | | |
| | | EPPK1 | | |
| TCGA_BRCA | AKT_pS473; | X4EBP1; | ACC_pS79; | AR; BCL2; GATA3; |
| | CKIT; CAVEOLIN1; | X4EBP1_pT37T46; | ACC1; CLAUDIN7; | HER2; IGFBP2; |
| | COLLAGENVI; | ASNS; ATM; CMYC; | ERALPHA; | INPP4B; FASN; |
| | EGFR_pY1068; | CASPASE7CLEAVEDD198; | BRAF; PDCD4; | EPPK1 |
| | FIBRONECTIN; | CYCLINB1; | PREX1; DUSP4 | |
| | GSK3ALPHABETA_pS21S9; | NFKBP65_pS536; | | |
| | HER2_pY1248; | S6_pS235S236; | | |
| | HSP70; MAPK_pT202Y204; | S6_pS240S244; | | |
| | PAI1; SRC_pY527; | STAT5ALPHA; SYK; | | |
| | MYH11; ANNEXIN1 | NDRG1_pT346; | | |
| | | P62LCKLIGAND; | | |
| | | P16INK4A | | |
| TCGA_CESC | CAVEOLIN1; | S6; S6_pS235S236; | CASPASE7CLEAVEDD198; | AMPKALPHA_pT172; |
| | CYCLINB1; | YAP; YAP_pS127; | MAPK_pT202Y204; | CLAUDIN7; |
| | EIF4G; | P62LCKLIGAND; | P38_pT180Y182; | ERALPHA; |
| | TRANSGLUTAMINASE; | MSH6; | PAI1; RAD51; | HER2; IGFBP2; INPP4B; |
| | EPPK1; ANNEXINI | P16INK4A | SRC_pY416; | SRC_pY527; |
| | | | NDRG1_pT346; RICTOR | RAB25 |
| TCGA_CHOL | ACC1; | A8N8; INPP4B; P53; | BAK; PAI1; TIGAR; | X53BP1; AMPK |
| | CASPASE7CLEAVEDD198; | SRC_pY416; | TRANSGLUTAMINASE; | ALPHA_pT172; |
| | CAVEOLIN1; | SRC_pY527; | ACETYLATEBULINLYS40 | ATM; CLAUDIN7; |
| | MEK1; | NDRG1_pT346; SCD1; | | ECADHERIN; |
| | PKCALPHA; | ANNEXIN1; | | GAB2; PEA15; |
| | PKCALPHA_pS657; | JAB1; MSH2 | | RAB25; RBM15; |
| | FASN; | | | EPPK1; P62LCKLIGAND; |
| | RICTOR; XBP1 | | | ADAR1; BRD4 |
| TCGA_COAD | EGFR_pY1068; | ASNS; BETACATENIN; | CAVEOLIN1; | CASPASE7CLEAVEDD198; |
| | NFKBP65_pS536; | CMYC; | COLLAGENVI; | CLAUDIN7; |
| | YB1; | CYCLINB1; | FIBRONECTIN; | EEF2; IGFBP2; |
| | RICTOR; TIGAR | ECADHERIN; | HER2; HSP70; | SRC_pY527; |
| | | INPP4B; RB_pS807S811; | MAPK_pT202Y204; | SYK; PDCD4; |
| | | S6; STAT5ALPHA; | PAI1; | TFRC; EPPK1; |
| | | RAB25; RBM15 | SRC_pY416; | ACETYLATUBULINLYS40; |
| | | | ETS1; MYH11; | DUSP4 |
| | | | NDRG1_pT346; | |
| | | | PEA15_pS116 | |
| TCGA_DLBC | ASNS; ATM; | CASPASE7CLEAVEDD198; | — | — |
| | BCL2; BIM; CYCLINB1; | NFKBP65_pS536; | | |
| | EEF2K; | P27; P53; S6_pS235S236; | | |
| | MEK1; PAI1; | SRC_pY416; STAT5ALPHA; | | |
| | RB_pS807S811; | ETS1; PREX1; | | |
| | SMAD1; SYK; | PDL1 | | |
| | CD20; CYCLINE2; | | | |
| | PDCD4; | | | |
| | PKCPANBETAII_pS660; | | | |
| | TFRC; XBP1; | | | |
| | P62LCKLIGAND; | | | |
| | ADAR1; MSH6 | | | |
| TCGA_ESCA | ASNS; | EGFR; PAI1; EIF4G; | CYCLINB1; | CLAUDIN7; HER2; |
| | CASPASE7CLEAVEDD198; | MYOSINIIA_pS1943; | IGFBP2; SRC_pY527; | HER2_pY1248; |
| | CAVEOLIN1; | NDRG1_pT346; | YAP_pS127; | S6_pS235S236; |
| | EGFR_pY1068; | EPPK1; | DUSP4 | SRC_pY416; |
| | FIBRONECTIN; | ANNEXIN1 | | RAB25; TIGAR; |
| | P38_pT180Y182; | | | XBP1; P62LCKLIGAND; |
| | RICTOR | | | P16INK4A |
| TCGA_GBM | X4EBP1_pT37T46; | X53BP1; | AKT_pT308; | ACC_pS79; |
| | CKIT; | AMPKALPHA_pT172; | FIBRONECTTN; | AKT_pS473; ASNS; |
| | RB_pS807S811; | ATM; | HER3; HSP70; | P70S6K_pT389; |
| | SRC_pY527; | BETACATENIN; EGFR; | IGFBP2; NFKBP65_pS536; | BRAF; GSK3_pS9 |
| | PEA15_pS116; | EGFR_pY1068; | PAI1; S6_pS235S236; | |
| | P16INK4A; | EGFR_2pY1173; | S6_pS240S244; | |
| | SHP2_pY542 | GSK3_ALPHABETA_pS21S9; | NDRG1_pT346 | |
| | | HER2_pY1248; | | |
| | | MAPK_pT202Y204; | | |
| | | PEA15; PKCALPHA; | | |
| | | PKCALPHA_pS657; PTEN; | | |
| | | SRC_pY416; | | |
| | | GAPDH; | | |
| | | PKCPANBETAII_pS660; | | |
| | | ACETYLATUBULINLYS40; | | |
| | | ANNEXIN1; | | |
| | | PREX1 | | |
| TCGA_HNSC | BAK; | CAVEOL1N1; | AKT_pT308; | AMPKALPHA_pT172; |
| | MAPK_pT202Y204; | EEF2; EGFR; | CASPASE7CLEAVEDD198; | ASNS; BETACATENIN; |
| | NFKBP65_pS536; | EGFR_pY1068; | PDCD4 | CLAUDIN7; |
| | RAD50; SRC_pY416; | EGFR_pY1173; | | CYCLINB1; |
| | SRC_pY527; | HER2_pY1248; HSP70; | | ECADHERIN; |
| | MYH11; | PAI1; PKCALPHA; | | IGFBP2; P53; |
| | NDRG1_pT346; | S6_pS235S236; | | EIF4G; GAPDH; TFRC |
| | EPPK1 | S6_pS240S244; | | |
| | | VEGFR2; | | |
| | | YAP_pS127; | | |
| | | ANNEX1N1; P16INK4A | | |
| TCGA_KICH | CKIT; CMYC; | AMPKALPHA; | — | — |
| | ERALPHA_pS118; | ASNS; CAVEOLIN1; | | |
| | ERK2; GAB2; | CLAUDIN7; | | |
| | P53; P70S6K_pT389; | ECADHERIN; | | |
| | GAPDH; | HER3; INPP4B; | | |
| | PKCPANBETAII_pS660; | MEK1; PAI1; | | |
| | RICTOR; | PDK1_pS241; | | |
| | ACETYLATUBULINLYS40; | PKCALPHA_pS657; | | |
| | MSH2; SMAC | SRC_pY416; | | |
| | | SRC_pY527; SYK; | | |
| | | MYH11; NDRG1_pT346; | | |
| | | BRAF_pS445; | | |
| | | P16INK4A | | |
| TCGA_KIRC | AKT_pS473; | NFKBP65_pS536; | X4EBP1_pT37T46; | — |
| | BAK; BETACATENIN; | P38_pT180Y182; | AKT_pT308; | |
| | CAVEOL1N1; | PRAS40_pT246; | PAI1; S6_pS235S236; | |
| | EGFR_pY1068; | TRANSGLUTAMINASE; | YAP_pS127; | |
| | EGFR_pY1173; GAB2; | P62LCKLIGAND; | G6PD; | |
| | HER3; HSP70; | CD26; | GAPDH; | |
| | MAPK_pT202Y204; | LDHB; | PEA15_pS116; | |
| | MIG6; S6_pS240S244; | MITOCHONDRIA; | ANNEXIN1; CA9; | |
| | SRC_pY527; | PKM2 | GYS; GYS_pS641; | |
| | VEGFR2; MYH11; | | LDHA | |
| | NDRG1_pT346; | | | |
| | RAB11; | | | |
| | RICTOR; EPPK1; | | | |
| | SHP2_pY542; | | | |
| | HIF1ALPHA; | | | |
| | PYGL | | | |
| TCGA_KIRP | AKT_pS473; | BAK; INPP4B; | NF2; GAPDH; | — |
| | AKT_pT308; | PAI1; MYH11; | TRANSGLUTAMINASE; | |
| | AMPKALPHA_pT172; | RICTOR; | SMAC | |
| | AR; ATM; | P62LCKLIGAND; | | |
| | CLAUDIN7; | P16INK4A | | |
| | HER2; HER3; | | | |
| | NFKBP65_pS536; | | | |
| | P38_pT180Y182; | | | |
| | PKCALPHA; | | | |
| | PKCALPHA_pS657; | | | |
| | SRC_pY527; GSK3_pS9; | | | |
| | NDRG1_pT346; | | | |
| | PKCPANBETAII_pS660; | | | |
| | EPPK1; | | | |
| | ACETYLATUBULINLYS40; | | | |
| | ANNEX1N1; CD26 | | | |
| TCGA_LGG | AKT_pS473; | X4EBP1_pT37T46; | ERK2; | GSK3ALPHABETA_pS21S9; |
| | BETACATENIN; | ACC_pS79; | MEK1; P70S6K_pT389; | MAPK_pT202Y204; |
| | EGFR; EGFR_pY1068; | ACC1; CKIT; | PEA15; | RAD51; SRC_pY527; |
| | EGFR_pY1173; | HER3; PKCALPHA; | PKCALPHA_pS657; | XBP1; P16INK4A |
| | HER2_pY1248; | PKCDELTA_pS664; | PTEN; | |
| | SMAD1; SRC_pY416; | ETS1; GSK3_pS9; | RB_pS807S811; | |
| | STATS_pY705; | SMAC | BRAF; | |
| | GAPDH; PEA15_pS116; | | NDRG1_pT346; | |
| | T1GAR; | | PKCPANBETAII_pS660; | |
| | PREX1; SHP2_pY542 | | ACETYLATUBULINLYS40 | |
| TCGA_LIHC | S6_pS235S236; | EGFR_pY1068; | ACC_pS79; | — |
| | S6_pS240S244; | IGFBP2; INPP4B: | ACC1; ASNS; | |
| | MYH11; | MAPK_pT202Y204; | FIBRONECTIN; | |
| | RAB25; | P53; | NFKBP65_pS536; | |
| | TRANSGLUTAMINASE | P70S6K_pT389; | PAI1; SRC_pY527; | |
| | | SRC_pY416; | P16INK4A | |
| | | FASN; | | |
| | | NDRG1_pT346; | | |
| | | P62LCKLIGAND; | | |
| | | MSH2 | | |
| TCGA_LUAD | CAVEOLIN1; | AMPKALPHA_pT172; | X4EBP1_pT37T46; | BETACATENIN; |
| | HSP70; P38_pT180Y182; | ASNS; | CKIT; | ECADHERIN; |
| | PAI1; | CASPASE7CLEAVEDD198; | CLAUDIN7; IGFBP2; | EGFR_pY1068; |
| | PKCALPHA_pS657; | INPP4B; | MAPK_pT202Y204; | EGFR_pY1173; HER2; |
| | VEGFR2; | NFKBP65_pS536; | S6_pS235S236; | HER2_pY1248; |
| | SMAC | STAT5ALPHA; | S6_pS240S244; | SRC_pY416; |
| | | G6PD; | SRC_pY527; MYH11; | NDRG1_pT346 |
| | | MYOSINIIA_pS1943; | PDCD4; RAB25; | |
| | | PKCPANBETAII_pS660; | ACETYLATUBULINLYS40; | |
| | | TRANSGLUTAMINASE; | DUSP4 | |
| | | EPPK1; | | |
| | | P62LCKLIGAND; | | |
| | | ANNEXIN1 | | |
| TCGA_LUSC | X4EBP1_pT37 | X4EBP1; ACC1; | CAVEOLIN1; | CASPASE7CLEAVEDD198; |
| | T46; AKT_pS473; | AKT_pT308; | MAPK_pT202Y204; | HER2_pY1248; |
| | BCL2; BETACATENIN; | ASNS; CLAUDIN7; | SRC_pY416; | HSP70; INPP4B; PAI1; |
| | CKIT; | CYCLINB1; ECADHERIN; | SYK; MYH11; | ANNEXIN1 |
| | GSK3ALPHABETA_pS21S9; | EEF2; | TRANSGLUTAMINASE | |
| | HER2; NFKBP65_pS536; | EGFR_pY1068; | | |
| | RB_pS807S811; | IGFBP2; G6PD; | | |
| | S6_pS240S244; | NDRG1_pT346; | | |
| | SRC_pY527; | TFRC; EPPK1; | | |
| | STAT5ALPHA; | P62LCKLIGAND | | |
| | BRAF; EIF4G; | | | |
| | GSK3_pS9; | | | |
| | RICTOR; | | | |
| | ACETYLATUBULINLYS40; | | | |
| | MSH6 | | | |
| TCGA_MESO | X4EBP1_pT37T46; | AMPKALPHA_pT172; | — | — |
| | ATM; COLLAGENVI; EGFR; | CAVEOLIN1; | | |
| | EGFR_pY1068; | FIBRONECTIN; | | |
| | HSP70; | MAPK_pT202Y204; | | |
| | IGFBP2; NFK | MEK1; P38_pT180Y182; | | |
| | BP65_pS536; | PAI1; PKCALPHA_pS657; | | |
| | P70S6K_pT389; | S6_pS235S236; | | |
| | PAXILLIN; | SRC_pY527; MYH11; | | |
| | STAT3_pY705; | PKCPANBETAII_pS660; | | |
| | YAP_pS127; | RICTOR; ANNEXIN1 | | |
| | HEREGULIN; | | | |
| | NDRG1_pT346; | | | |
| | PDCD4; P16INK4A | | | |
| TCGA_OV | FIBRONECTIN; | X4EBP1_pT37T46; | AMPKALPHA_pT172; | ASNS; CYCLINB1; |
| | INPP4B; | ATM; BETACATENIN; | AR; CMYC; | MEK1_pS217S221; |
| | MAPK_pT202Y204; | BIM; GAB2; | CLAUDIN7; | PKCPANBETAII_pS660; |
| | NFKBP65_pS536; | RICTOR; EPPK1; | CYCLINE1; | P16INK4A |
| | P38_pT180Y182; | MSH6; BRD4 | ECADHERIN; | |
| | PAI1; | | ERALPHA; | |
| | S6_pS235S236; | | HER2; HSP70; | |
| | S6_pS240S244; | | IGFBP2; RB_pS807S811; | |
| | SRC_pY527; | | SYK; YAP_pS127; | |
| | STAT5ALPHA; | | ACETYLATUBULINLYS40 | |
| | MYH11; | | | |
| | NDRG1_pT346; | | | |
| | ANNEXIN1 | | | |
| TCGA_PAAD | AMPKALPHA_pT172; | CMYC; CAVEOLIN1; | — | — |
| | CLAUDIN7; HSP70; | CDK1; | | |
| | IGFBP2; INPP4B; | FIBRONECTIN; | | |
| | SYK; VEGFR2; | MAPK_pT202Y204; | | |
| | PDCD4; | NFKBP65_pS536; | | |
| | PKCPANBETAII_pS660; | P38_pT180Y182; | | |
| | RAB25; | PAI1; SRC_pY527; | | |
| | EPPK1; | YAP_pS127; | | |
| | P16INK4A | MYH11; | | |
| | | NDRG1_pT346; | | |
| | | RICTOR; ANNEXIN1; | | |
| | | SMAC | | |
| TCGA_PCPG | X4EBP1_pT37T46; | X53BP1; | — | — |
| | CRAF_pS338; | AKT_pS473; BAK; | | |
| | CDK1; | CAVEOLIN1; IGFBP2; | | |
| | EGFR_pY1173; | INPP4B; PAI1; | | |
| | GAB2; P38_pT180Y182; | PAXILLIN; RAD51; | | |
| | PKCALPHA; | RB_pS807S811; | | |
| | PKCALPHA_pS657; | BRAF; RICTOR; | | |
| | CASPASE8; | XBP1; | | |
| | MSH2 | ACETYLATUBULINLYS40; | | |
| | | P16INK4A | | |
| TCGA_PRAD | AKT_pS473; | ACC_pS79; ACC1; | — | — |
| | CAVEOLIN1; ERK2; | AKT_pT308; | | |
| | GSK3ALPHABETA_pS21S9; | AR; BETACATENIN; | | |
| | HSP70; | CLAUDIN7; | | |
| | INPP4B; LCK; | ECADHERIN; | | |
| | MAPK_pT202Y204; | IGFBP2; PTEN; | | |
| | P38_pT180Y182; | FASN; GSK3_pS9; | | |
| | P70S6K_pT389; | NDRG1_pT346; | | |
| | PKCALPHA; | PEA15_pS116; | | |
| | PKCALPHA_pS657; | RAB25; | | |
| | SRC_pY527; | TRANSGLUTAMINASE; | | |
| | STAT3_pY705; | EPPK1 | | |
| | YAP_pS127; MYH11; | | | |
| | PKCPANBETAII_pS660; | | | |
| | RICTOR | | | |
| TCGA_READ | CASPASE7CLEAVEDD198; | CAVEOLIN1; | X4EBP1_pT37T46; | ACC_pS79; ACC1; |
| | HER2_pY1248; | COLLAGENVI; | ASNS; | CYCLINB1; |
| | IGFBP2 | FIBRONECTIN; | BETACATENIN; | INPP4B; SYK; RAB25; |
| | | HSP70; P53; PAI1; | CMYC; CLAUDIN7; | RBM15; TFRC; |
| | | ETS1; MYH11; | ECADHERIN; | EPPK1 |
| | | RICTOR; TIGAR | EEF2; | |
| | | | EGFR_pY1068; | |
| | | | HER2; | |
| | | | MAPK_pT202Y204; | |
| | | | NFKBP65_pS536; | |
| | | | PTEN; RB_pS807S811; | |
| | | | SRC_pY527; | |
| | | | NDRG1_pT346; | |
| | | | PDCD4; | |
| | | | PEA15_pS116 | |
| TCGA_SARC | X4EBP1_pT37T46; | CASPASE7CLEAVEDD198; | X53BP1; ATM; | CKIT; CYCLINB1; |
| | AKT_pS473; | RAD51; | IGFBP2; P70S6K1; | FIBRONECTIN; |
| | AKT_pT308; | SYK; | XBP1 | PAI1; PAXILLIN; |
| | AR; CAVEOLIN1; | ANNEXIN1; | | PKCALPHA; |
| | CYCLINE1; | PREX1 | | SRC_pY416; |
| | GAB2; | | | SRC_pY527; |
| | GSK3ALPHABETA_pS21S9; | | | MYOSINIIA_pS1943 |
| | MAPK_pT202Y204; | | | |
| | MEK1; NFKBP65_pS536; | | | |
| | P38_pT180Y182; | | | |
| | YAP_pS127; | | | |
| | GSK3_pS9; | | | |
| | MYH11; NDRG1_pT346; | | | |
| | RAB25; | | | |
| | RICTOR; | | | |
| | EPPK1; P62LCKLIGAND; | | | |
| | P16INK4A | | | |
| TCGA_SKCM | X4EBP1_pT37T46; | AKT_pS473; | — | — |
| | AKT; BCL2; | AKT_pT308; ATM; | | |
| | CKIT; EEF2; | CASPASE7CLEAVEDD198; | | |
| | GAB2; MAPK_pT202Y204; | CAVEOLIN1; | | |
| | PAXILLIN; PTEN; | COLLAGENV1; | | |
| | RAD50; | FIBRONECTIN; | | |
| | RB_pS807S811; | GSK3ALPHABETA_pS21S9; | | |
| | SRC_pY527; | HER3; HSP70; | | |
| | STAT5ALPHA; | PAI1; PKCALPHA; | | |
| | P62LCKLIGAND; | PKCALPHA_pS657; | | |
| | BRD4; DUSP4 | S6_pS235S236; | | |
| | | S6_pS240S244; | | |
| | | GAPDH; | | |
| | | GSK3_pS9; | | |
| | | NDRG1_pT346; | | |
| | | TFRC; ANNEXIN1; | | |
| | | P16INK4A | | |
| TCGA_STAD | CASPASE7CLEAVEDD198; | ASNS; BETACATENIN; | — | — |
| | CAVEOLIN1; | CLAUDIN7; | | |
| | ERK2; HSP70; | CYCLINB1; | | |
| | P27; SRC_pY527; | CYCLINE1; | | |
| | STAT5ALPHA; | EGFR_pY1068; | | |
| | CD20; ETS1; | HER2; PAI1; RAD50; | | |
| | MYH11; PEA15_pS116; | G6PD; MYOSINIIA_pS1943; | | |
| | RICTOR; | NDRG1_pT346; | | |
| | TRANSGLUTAMINASE; | RAB25; TIGAR; TFRC; | | |
| | ERCC1 | EPPK1; SMAC; | | |
| | | DUSP4; P16INK4A | | |
| TCGA_TGCT | ASNS; CYCLINB1; | AR; CKIT; | CAVEOLIN1; | — |
| | HER3; PAI1; | CASPASE7CLEAVED | HSP70; IGFBP2; | |
| | SRC_pY416; | D198; CHK2; | PKCALPHA; | |
| | FASN; EPPK1 | CHK2_pT68; | PKCALPHA_pS657; | |
| | | EEF2K; GAB2; KU80; | SRC_pY527; | |
| | | MTOR; S6; | YAP_pS127; | |
| | | STAT5ALPHA; | RICTOR; XBP1 | |
| | | SYK; | | |
| | | CYCLINE2; PDCD4; | | |
| | | PRDX1; PREX1; | | |
| | | ADAR1; MSH2; | | |
| | | MSH6 | | |
| TCGA_THCA | FTBRONECTIN; | AKT_pS473; | BETACATENIN; | — |
| | HSP70; | BCL2; CKIT; CLAUDIN7; | CAVEOLIN1; | |
| | MYH11; RAB25; | COLLAGENVI; | ERK2; | |
| | SCD1; ANNEXIN1; | ECADHERIN; | P38_pT180Y182; | |
| | PREX1; SMAC; | EGFR_pY1068; | RAD50; STAT3_pY705; | |
| | CDK1_pY15; | HER2_pY1248; | YAP_pS127; | |
| | DUSP4 | LKB1; MAPK_pT202Y204; | PDL1 | |
| | | S6_pS240S244; | | |
| | | SRC_pY527; BRCA2; | | |
| | | ETS1; PDCD4; | | |
| | | PEA15_pS116; | | |
| | | PKCPANBETAII_pS660; | | |
| | | TIGAR | | |
| TCGA_THYM | CKIT; | X4EBP1_pT37T46; | CAVEOLIN1; | — |
| | CASPASE7CLEAVEDD198; | CYCL1NB1; | CD49B; DVL3; | |
| | EEF2K | GAB2; GATA3; | ECADHERIN; | |
| | | GSK3ALPHABETA_pS21S9; | MAPK_pT202Y204; | |
| | | LCK; NFKBP65_pS536; | PAXILLIN; | |
| | | P38_pT180 | SRC_pY527; | |
| | | Y182; PCNA; | NDRG1_pT346; | |
| | | RB_pS807S811; | PDCD4; EPPK1; | |
| | | SMAD1; SRC_pY416; | XBP1; | |
| | | STAT5ALPHA; | P62LCKLIGAND | |
| | | STATHMIN; | | |
| | | ETS1; GSK3_pS9; | | |
| | | RBM15; MSH2; | | |
| | | MSH6 | | |
| TCGA_UCEC | AKT_pS473; | BETACATENIN; | ACC1; | — |
| | AKT_pT308; AR; | ECADHERIN; | AMPKALPHA_pT172; | |
| | BCL2; | EEF2; ERALPHA_pS118; | ASNS; | |
| | CASPASE7CLEAVEDD198; | GAPDH; PDCD4; | CLAUDIN7; | |
| | CAVEOLIN1; | PEA15_pS116; | CYCLINB1; CYCLINE1; | |
| | ERALPHA; GSK3AL | ACETYLATUBULINLYS40 | HER2; IGFBP2; | |
| | PHABETA_pS21S9; | | P53; | |
| | MAPK_pT202Y204; | | RB_pS807S811; | |
| | P38_pT180Y182; | | NDRG1_pT346; TFRC; | |
| | PKCALPHA; | | EPPK1; P16INK4A | |
| | PKCALPHA_pS657; | | | |
| | PTEN; | | | |
| | SYK; GSK3_pS9; | | | |
| | MYH11; RICTOR; | | | |
| | TRANSGLUTAMINASE; | | | |
| | ANNEXIN1 | | | |
| TCGA_UCS | X4EBP1_pT37T46; | AKT_pS473; ASNS; | — | — |
| | X53BP1; | GSK3ALPHABETA_pS21S9; | | |
| | ACC_pS79; | NFKBP65_pS536; | | |
| | ATM; FIBRONECTIN; | PAI1; RAD51; | | |
| | GAB2; HER2; | NDRG1_pT346 | | |
| | IGFBP2; P70S6K1; | | | |
| | PAXILLIN; | | | |
| | RAD50; | | | |
| | S6; SRC_pY527; | | | |
| | HEREGULIN; | | | |
| | XBP1; | | | |
| | ACETYLATUBULINLYS40; | | | |
| | MSH2; MSH6; | | | |
| | BRD4; | | | |
| | P16INK4A; PDL1 | | | |
| TCGA_UVM | X4EBP1_pT37T46; | EEF2K; | ACC_pS79; | — |
| | BETACATEN1N; | GSK3ALPHABETA_pS21S9; | ACC1; | |
| | ECADHERIN; | HER3; P38_pT180Y182; | AMPKALPHA_pT172; | |
| | GAB2; | RAD51; SRC_pY527; | ATM; BAK; CKIT; | |
| | PAXILLIN; | YAP_pS127; | INPP4B; LCK; | |
| | ACETYLATUBULINLYS40; | BAP1C4; GSK3_pS9; | NFKBP65_pS536; | |
| | ADAR1; | NDRG1_pT346; | PKCALPHA; | |
| | CDK1_pY15 | P21; PDCD4; | PKCALPHA_pS657; | |
| | | TUBERIN_pT1462; | PTEN; SRC_pY416; | |
| | | CASPASE8; | RBM15; | |
| | | DUSP4; ERCC5 | P62LCKLIGAND; | |
| | | | PREX1 | |

### Example 10: Alternative/Secondary AHR Activation Signatures for 32 Cancer Types-2

The inventors have determined alternative (secondary) AHR activation signatures based on proteomics (Reverse Phase Protein Array (RPPA)) data using NMF clustering as shown in Table 6. These alternative AHR activation signatures can be used to determine the AHR activation status of a sample using protein biomarkers listed in Table 6.

Table 6: Tabular representation of the different RPPA features that could be used to call AHR activation for the 32 TCGA cancers divided among the different AHR subgroups for each cancer entity defined by consensus NMF clustering.

| | g1 | g2 | g3 | g4 | g5 | g6 |
|---|---|---|---|---|---|---|
| TCGA_ACC | INPP4B; | ACC_pS79; | — | — | — | — |
| | NFKBP65_pS536; | ACC1; | | | | |
| | PAI1; | AKT; | | | | |
| | PKCALPHA; | AKT_pS473; | | | | |
| | PKCALPHA_pS657; | AMPKALPHA_pT172; | | | | |
| | STAT5ALPHA; | ATM; | | | | |
| | G6PD; | CKIT; | | | | |
| | ACETYLATUBULINLYS40; | CYCLINE1; | | | | |
| | ANNEXIN1; | EEF2K; | | | | |
| | PREX1; | GSK3ALPHABETA_pS21S9; | | | | |
| | SMAC | MEK1; | | | | |
| | | BRAF; GAPDH; | | | | |
| | | NDRG1_pT346; | | | | |
| | | PKCPANBETAII_pS660; | | | | |
| | | RAPTOR; BRD4 | | | | |
| TCGA_BLCA | ACC_pS79; | AMPKALPHA_pT172; | CASPASE7CLEAVEDD198; | X4EBP1_pT37T46; | — | — |
| | ACC1; | ATM; | CYCLINB1; | CKTT; | | |
| | CLAUDIN7; | CAVEOLIN1; | EGFR; PAI1; | P38_pT180Y182; | | |
| | ECADHERIN; | FIBRONECTIN; | G6PD; NDRG1_pT346; | EPPK1 | | |
| | EGFR_pY1068; | HSP70; | TFRC; P62LCKLIGAND; | | | |
| | GATA3; | VEGFR2; | ANNEXIN1; | | | |
| | HER2; | YAP_pS127; | P16INK4A | | | |
| | HER2_pY1248; | MYH11; | | | | |
| | HER3; | RICTOR; | | | | |
| | INPP4B; | TRANSGLUTAMINASE | | | | |
| | RB_pS807S811; | | | | | |
| | SRC_pY527; | | | | | |
| | BRAF; | | | | | |
| | FASN; | | | | | |
| | RAB25; | | | | | |
| | MSH6 | | | | | |
| TCGA_BRCA | ACC_pS79; | X4EBP1; | PDCD4; | AR; EGFR_pY1068; | ART_pS473; | — |
| | ACC1; | X4EBP1_pT37T46; | EPPK1 | FIBRONECTIN; | CMYC; | |
| | BCL2; CLAUDIN7; | ASNS; | | HER2; | CAVEOLIN1; | |
| | ECADHERIN; | ATM; CKIT; | | HER2_pY1248; | COLLAGENVI; | |
| | ERALPHA; | CASPASE7CLEAVEDD198; | | FASN | HSP70; | |
| | GATA3; GSK3 | CYCLINB1; NFK | | | MAPK_pT202Y204; | |
| | ALPHABETA_pS21S9; | BP65_pS536; | | | PAI1; | |
| | IGFBP2; | S6_pS235S236; | | | SRC_pY527; | |
| | INPP4B; | S6_pS240S244; | | | MYH11; | |
| | BRAF; | STAT5ALPHA; | | | ANNEXIN1 | |
| | GSK3_pS9; | SYK; | | | | |
| | PREX1; | NDRG1_pT346; | | | | |
| | DUSP4 | P62LCKLIGAND; | | | | |
| | | P16INK4A | | | | |
| TCGA_CESC | AMPKALPHA_pT172; | CYCLINB1; | CASPASE7CLEAVEDD198; | S6; | MAPK_pT202Y204; | — |
| | CLAUDIN7; | ECADHERIN; | CAVEOLIN1; | S6_pS235S236; | P38_pT180Y182; | |
| | ERALPHA; | EIF4G; | PAI1; | FASN; MSH6 | SRC_pY416; | |
| | IGFBP2; | RAB25; | RAD51; YAP; | | SRC_pY527; | |
| | INPP4B | EPPK1; | YAP_pS127; | | NDRG1_pT346; | |
| | | P62LCKLIGAND | TRANSGLUTAMINASE; | | PDCD4; | |
| | | | ANNEXIN1; | | RICTOR | |
| | | | P16INK4A | | | |
| TCGA_CHOL | ASNS; | CASPASE7CLEAVEDD198; | ACC1; | X53BP1; | — | — |
| | P38_pT180Y182; | INPP4B; | BAK; FASN; | AMPKALPHA_pT172; | | |
| | PAI1; | MEK1; P53; | RICTOR; | ATM; CLAUDIN7; | | |
| | SRC_pY416; | PKCALPHA; | TIGAR; | ECADHERIN; | | |
| | SRC_pY527; | PKCALPHA_pS657; | TRANSGLUTAMINASE; | GAB2; PEA15; | | |
| | NDRG1_pT346; | ACETYLATUBULINLYS40 | XBP1 | RAB25; | | |
| | SCD1; | | | RBM15; | | |
| | ANNEXIN1; | | | EPPK1; P62LC | | |
| | JAB1; | | | KLIGAND; | | |
| | MSH2 | | | ADAR1; | | |
| | | | | BRD4 | | |
| TCGA_COAD | CASPASE7CLEAVEDD198; | ASNS; | CAVEOLIN1; | X4EBP1_pT37T46; | CLAUDIN7; | — |
| | EEF2; | BETACATENIN; | COLLAGENVI; | EGFR_pY1068; | HER2; IGFBP2; | |
| | SYK; GAPDH; | CMYC; | FIBRONECTIN; | NFKBP65_pS536; | MAPK_pT202Y204; | |
| | PEA15_pS116; | CYCL1NB1; | HSP70; | RB_pS807S811; | SRC_pY416; | |
| | TIGAR; | ECADHERIN; | PAI1; | YB1 | SRC_pY527; | |
| | TFRC; | INPP4B; S6; | ETS1; MYHH; | | NDRG1_pT346; | |
| | EPPK1; | STAT5ALPHA; | RICTOR | | PDCD4 | |
| | ACETYLATUBULINLYS40 | RAB25; | | | | |
| | | RBM15 | | | | |
| TCGA_DLBC | X4EBP1_pT37T46; | ATM; BIM; | ASNS; | BCL2; HER3; | — | — |
| | GSK3ALPHABETA_pS21S9; | MEK1; | CASPASE7CLEAVEDD198; | MAPK_pT202Y204; | | |
| | NFKBP65_pS536; | SMAD1; | CAVEOLIN1; | PKCALPHA_pS657; | | |
| | P27; P38_pT180Y182; | CD20; | CYCLINB1; | RB_pS807S811; | | |
| | S6_pS235S236; | TFRC; XBP1; | EEF2K; | SYK; CYCLINE2; | | |
| | S6_pS240S244; | P62LCKLIGAND; | P53; PAI1; | PDCD4 | | |
| | SRC_pY416; | ADAR1; | ETS1; PDL1 | | | |
| | SRC_pY527; | MSH6 | | | | |
| | STAT5ALPHA; | | | | | |
| | GSK3_pS9; | | | | | |
| | PKCPANBETAII_pS660; | | | | | |
| | PREX1 | | | | | |
| TCGA_ESCA | CAVEOLIN1; | CLAUDIN7; | CASPASE7CLEAVEDD198; | ASNS; | — | — |
| | FIBRONECTIN; | EGFR_pY1068; | CYCLINB1; | RICTOR | | |
| | PAI1; | HER2; | EGFR; IGFBP2; | | | |
| | NDRG1_pT346; | HER2_pY1248; | MYOSINIIA_pS1943; | | | |
| | ANNEXIN1 | P38_pT180Y182; | EPPK1 | | | |
| | | S6_pS235S236; | | | | |
| | | SRC_pY416; | | | | |
| | | SRC_pY527; | | | | |
| | | YAP_pS127; | | | | |
| | | RAB25; | | | | |
| | | TIGAR; XBP1; | | | | |
| | | P62LCKLIGAND; | | | | |
| | | DUSP4; | | | | |
| | | P16INK4A | | | | |
| TCGA_GBM | X4EBP1_pT37T46; | X53BP1; | AKT_pT308; | AKT_pS473; | — | — |
| | ACC_pS79; | AMPKALPHA_pT172; | FIBRONECTIN; | GSK3ALPHABETA_pS21S9; | | |
| | ASNS; | ATM; | HER3; | MAPK_pT202Y204; | | |
| | CKIT; | BETACATENIN; | HSP70; IGFBP2; | P70S6K_pT389; | | |
| | RB_pS807S811; | EGFR; | NFKBP65_pS536; | PEA15; | | |
| | SRC_pY527; | EGFR_pY1068; | PAI1; | PKCPANBETAII_pS660; | | |
| | BRAF; | EGFR_pY1173; | S6_pS235S236; | ACETYLATUBULINLYS40 | | |
| | GSK3_pS9; | HER2_pY1248; | S6_pS240S244; | | | |
| | P16INK4A; | PKCALPHA; | NDRG1_pT346; | | | |
| | SHP2_pY542 | PKCALPHA_pS657; | ANNEXIN1 | | | |
| | | PTEN; | | | | |
| | | SRC_pY416; | | | | |
| | | GAPDH; | | | | |
| | | PREX1 | | | | |
| TCGA_HNSC | CAVEOLIN1; | BAK; | AKT_pT308; | AMPKALPHA_pT172; | — | — |
| | EEF2; | NFKBP65_pS536; | CASPASE7CLEAVEDD198; | ASNS; | | |
| | EGFR; | P53; | PAI1 | BETACATEN1N; | | |
| | EGFR_pY1068; | RAD50; | | CLAUDIN7; | | |
| | EGFR_pY1173; | MYH11 | | CYCLINB1; | | |
| | HER2_pY1248; HSP70; | | | ECADHERIN; | | |
| | MAPK_pT202Y204; | | | IGFBP2; | | |
| | PKCALPHA; | | | EIF4G; PDCD4; | | |
| | S6_pS235S236; | | | TFRC | | |
| | S6_pS240S244; | | | | | |
| | SRC_pY416; SRC_pY527; | | | | | |
| | VEGFR2; | | | | | |
| | YAP_pS127; NDRG1_pT346; | | | | | |
| | EPPK1; | | | | | |
| | ANNEXIN1; | | | | | |
| | P161NK4A | | | | | |
| TCGA_KICH | CLAUDIN7; | CK1T; | ECADHERIN; | AMPKALPHA; | — | — |
| | HER3; | CMYC; P53; | ERALPHA_pS118; | ASNS; | | |
| | INPP4B; | P70S6K_pT389; | ERK2; GAB2; | CAVEOLIN1; | | |
| | MEK1; | GAPDH; | YAP_pS127; | PAI1; PDK1_pS241; | | |
| | PKCALPHA_pS657; | MSH2; | RICTOR; | SYK; MYH11; | | |
| | SRC_pY416; | SMAC | ACETYLATUBULINLYS40 | PKCPANBETAII_pS660; | | |
| | SRC_pY527; | | | BRAF_pS445; | | |
| | NDRG1_pT346 | | | P16INK4A | | |
| TCGA_KIRC | AKT_pS473; | TRANSGLUTAMINASE; | BAK; CAVEOL1N1; | X4EBP1_pT37T46; | — | — |
| | AMPKALPHA_pT172; | LDHB; | FIBRONECTIN; | AKT_pT308; | | |
| | BETACATENIN; | MITOCHONDRIA | HSP70; | P38_pT180Y182; | | |
| | EGFR_pY1068; | | PAI1; | S6_pS235S236; | | |
| | EGFR_pY1173; | | VEGFR2; | YAP_pS127; | | |
| | GAB2; HER3; | | MYH11; | G6PD; | | |
| | MAPK_pT202Y204; | | RICTOR; | GAPDH; | | |
| | MIG6; | | ANNEXIN1; | PEA15_pS116; | | |
| | NFKBP65_pS536; | | HIF1ALPHA | P62LCKLIGAND; | | |
| | PRAS40_pT246; | | | CA9; GYS; | | |
| | S6_pS240S244; | | | GYS_pS641; | | |
| | SRC_pY527; | | | LDHA; | | |
| | NDRG1_pT346; | | | PKM2 | | |
| | RAB11; | | | | | |
| | EPPK1; | | | | | |
| | CD26; | | | | | |
| | SHP2_pY542; | | | | | |
| | PYGL | | | | | |
| TCGA_KIRP | NF2; | BAK; INP | ART_pS473; | — | — | — |
| | GAPDH; | P4B; PAI1; | AKT_pT308; | | | |
| | TRANSGLUTAMINASE; | SYK; | AMPKALPHA_pT172; | | | |
| | SMAC | MYH11; | AR; ATM; | | | |
| | | RICTOR; | CLAUDIN7; | | | |
| | | P62LCKLIGAND; | GSK3ALPHABETA_pS21S9; | | | |
| | | P16INK4A | HER2; | | | |
| | | | HER3; | | | |
| | | | NFKBP65_pS536; | | | |
| | | | P38_pT180Y182; | | | |
| | | | PKCALPHA; | | | |
| | | | PKCALPHA_pS657; | | | |
| | | | SRC_pY527; | | | |
| | | | GSK3_pS9; | | | |
| | | | NDRG1_pT346; | | | |
| | | | PKCPA_NBETAII_pS660; | | | |
| | | | EPPK1; | | | |
| | | | ACETYLATUBULINLYS40; | | | |
| | | | ANNEXIN1; | | | |
| | | | CD26 | | | |
| TCGA_LGG | MEK1; | X4EBP1_pT37T46; | AKT_pS473; | ERK2; | PKCDELTA_pS664; | — |
| | P70S6K_pT389; | ACC_pS79; | BETACATENIN; | GSK3ALPHABETA_pS21S9; | ETS1; | |
| | PKCALPHA_pS657; | ACC1; | EGFR; | MAPK_pT202Y204; | PEA15_pST16; | |
| | PTEN; | CKIT; | EGFR_pY1068; | PEA15; | XBP1; | |
| | BRAF; | HER3; | EGFR_pY1173; | RAD51; SRC_pY416; | P16INK4A | |
| | NDRG1_pT346; | PKCALPHA; | HER2_pY1248; | SRC_pY527; | | |
| | PKCPANBETAII_pS660; | RB_pS807S811; | SMAD1; | SHP2_pY542 | | |
| | ACETYLATUBULINLYS40 | GSK3_pS9; | STAT3_pY705; | | | |
| | | SMAC | TIGARPREX1 | | | |
| TCGA_L1HC | ACC_pS79; | X4EBP1_pT37T46; | INPP4B; | P62LCKLIGAND | — | — |
| | ACC1; | ASNS; | MAPK_pT202Y204; | | | |
| | AKT_pS473; | FIBRONECTIN; | S6_pS235S236; | | | |
| | EGFR_pY1068; | PAI1; | MYH11; | | | |
| | IGFBP2; | PAXILLIN; | RAB25; | | | |
| | NFKBP65_pS536; | VEGFR2; | TRANSGLUTAMINASE | | | |
| | P53; | P16INK4A | | | | |
| | P70S6K_pT389; | | | | | |
| | S6_pS240S244; | | | | | |
| | SRC_pY416; | | | | | |
| | SRC_pY527; | | | | | |
| | FASN; | | | | | |
| | NDRG1_pT346; | | | | | |
| | MSH2 | | | | | |
| TCGA_LUAD | CASPASE7CLEAVEDD198; | X4EBP1_pT37T46; | ASNS; BETACATENIN; | AMPKALPHA_pT172; | — | — |
| | HSP70; | SRC_pY416; | CKIT; CLAUDIN7; | CAVEOLIN1; | | |
| | INPP4B; | NDRG1_pT346; | ECADHERIN; EGFR_pY1173; | EGER_pY1068; | | |
| | PAI1; | EPPK1; | HER2; IGFBP2; | HER2_pY1248; | | |
| | STAT5ALPHA; | P62LCKLIGAND; | G6PD; | MAPK_pT202Y204; | | |
| | ANNEXIN1 | SMAC | RAB25; | NFKBP65_pS536; | | |
| | | | ACETYLATUBULINLYS40; | P38_pT180Y182; | | |
| | | | DUSP4 | PKCALPHA_pS657; | | |
| | | | | S6_pS235S236; | | |
| | | | | S6_pS240S244; | | |
| | | | | SRC_pY527; | | |
| | | | | VEGFR2; | | |
| | | | | MYH11; PDCD4; | | |
| | | | | PKCPANBETAII_pS660; | | |
| | | | | TRANSGLUTAM1NASE | | |
| TCGA_LUSC | CASPASE7CLEAVEDD198 | X4EBP1; | X4EBP1_pT37T46; | CAVEOLIN1; | HSP70; | — |
| | | ACC1; | AKT_pS473; | HER2_pY1248; | MAPK_pT202Y204; | |
| | | ASNS; | AKT_pT308; | INPP4B; PAI1; | NFKBP65pS536; | |
| | | BETACATENIN; | BCL2; | TRANSGLUTAMINASE; | SRC_pY416; | |
| | | CLAUDIN7; | CKIT; | ANNEXIN1 | SRC_pY527; | |
| | | ECADHERIN; | CYCLINB1; | | STAT5ALPHA; | |
| | | EEF2; | GSK3ALPHABETA_pS21S9; | | SYK; | |
| | | EGFR_pY1068; | HER2; | | MYH11 | |
| | | IGFBP2; | RB_pS807S811; | | | |
| | | EIF4G; | S6_pS240S244; | | | |
| | | G6PD; | BRAF; GSK3_pS9; | | | |
| | | NDRG1_pT346; | RICTOR; | | | |
| | | TFRC; | ACETYLATUBULINLYS40; | | | |
| | | EPPK1; | MSH6 | | | |
| | | P62LCKLIGAND | | | | |
| TCGA_MESO | AMPKALPHA_pT172; | FIBRONECTIN; | X4EBP1_pT37T46; | ATM; COL | — | — |
| | ASNS; | GSK3ALPHABETA | EGFR; | LAGENVI; | | |
| | CASPASE7CLEAVEDD198; | pS21S9; | EGFR_pY1068; | YAP_pS127; | | |
| | CAVEOLIN1; | HSP70; | IGFBP2; | HEREGULIN; | | |
| | MEK1; | MAPK_pT202Y204; | SRC_pY416; | P16INK4A | | |
| | P70S6K_pT389; | NFKBP65pS536; | STAT3_pY705; | | | |
| | PAI1; | P38_pT180Y182; | NDRG1_pT346; | | | |
| | S6_pS235S236; | PAXILLIN; | PDCD4; | | | |
| | MYH11; | PKCALPHA_pS657; | RICTOR | | | |
| | PKCPANBETAII_pS660; | SRC_pY527 | | | | |
| | P62LCKLIGAND; | | | | | |
| | ANNEXIN1 | | | | | |
| TCGA_OV | FTBRONECTIN; | AMPKALPHA_pT172; | X4EBP1_pT37T46; | — | — | — |
| | HSP70; | ASNS; | AR; ATM; | | | |
| | MAPK_pT202Y204; | CYCLINB1; | BETACATENIN; | | | |
| | MEK1_pS217S221; | CYCLINE1; | BIM; | | | |
| | NFKBP65_pS536; | ERALPHA; | CMYC; | | | |
| | P38_pT180Y182; | GAB2; | CLAUDIN7; | | | |
| | PAI1; | IGFBP2; | ECADHERIN; | | | |
| | S6_pS235S236; | STAT5ALPHA; | HER2; | | | |
| | S6_pS240S244; | SYK; | RB_pS807S811; | | | |
| | SRC_pY527; | PKCPAXBETAII_pS660; | RICTOR; | | | |
| | YAP_pS127; | EPPK1; | ACETYLATUBULINLYS40; | | | |
| | MYH11; | P16INK4A | MSH6; | | | |
| | NDRG1_pT346; | | BRD4 | | | |
| | ANNEXIN1 | | | | | |
| TCGA_PAAD | CAVEOLIN1; | AMPKALPHA_pT172; | CMYC; | HSP70; SYK; | X4EBP1; | FLBRONECTIN; |
| | MAPK_pT202Y204; | ECADHERIN; | SMAC; | ANNEXIN1 | X4EBP1_pT37T46; | NFKBP65_pS536; |
| | P38_pT180Y182; | INPP4B; | P16INK4A | | AKT_pS473; | PAI1 |
| | SRC_pY527; | VEGFR2; | | | ASNS; | |
| | YAP_pS127; | RAB25; | | | ATM; | |
| | MYH11; | EPPK1 | | | BETACATENIN; | |
| | RICTOR | | | | CDK1; | |
| | | | | | CLAUDIN7; | |
| | | | | | GSK3ALPHABETA_pS21S9; | |
| | | | | | IGFBP2; | |
| | | | | | S6_pS235S236; | |
| | | | | | NDRG1_pT346; | |
| | | | | | PDCD4; | |
| | | | | | PKCPANBETAII_pS660; | |
| | | | | | ACETYLATUBULINLYS40 | |
| TCGA_PCPG | X4EBP1_pT37T46; | X53BP1; | ART_pS473; | — | — | — |
| | BAK; CDR1; | ATM; | CRAF_pS338; | | | |
| | PRCALPHA; | GAB2; INPP4B; | CAVEOLIN1; | | | |
| | PKCALPHA_pS657; | P38_pT180Y182; | EGFR_pY1173; | | | |
| | RB_pS807S811; | ACETYLATUBULINLYS40; | IGFBP2; PA11; | | | |
| | CASPASE8 | MSH2 | PAXILLIN; | | | |
| | | | RAD51; | | | |
| | | | BRAF; | | | |
| | | | RICTOR; XBP1; | | | |
| | | | P16INK4A | | | |
| TCGA_PRAD | ATM; HSP70; | ACC_pS79; | HER2; | CAVEOLIN1; | ART_pS473; | — |
| | IGFBP2; | ACC1; | PKCALPHA_pS657; | ERK2; | ART_pT308; | |
| | LCK; ETS1; | AR; | YAP_pS127 | PKCALPHA; | GSK3ALPHABETA_pS21S9; | |
| | PEA15_pS116; | BETACATESIN; | | PKCPANBETAII_pS660; | MAPK_pT202Y204; | |
| | TRANSGLUTAMINASE | CLAUDIN7; | | RAB25 | P38_pT180Y182; | |
| | | ECADHERIN; | | | P70S6K_pT389; | |
| | | INPP4B; PTEN; | | | SRC_pY527; | |
| | | FASN; | | | STAT3_pY705; | |
| | | NDRG1_pT346; | | | GSK3_pS9; | |
| | | PDCD4 | | | MYH11; | |
| | | | | | RICTOR; | |
| | | | | | EPPK1 | |
| TCGA_READ | X4EBP1_pT37T46; | CASPASE7CLEAVEDD198; | ACC_pS79; | — | — | — |
| | BETACATENIN; | COLLAGENVI; | ACC1; ASNS; | | | |
| | CMYC; | FIBRONECTIN; | CYCLINB1; | | | |
| | CAVEOLIN1; | HSP70; | HER2_pY1248; | | | |
| | CLAUDIN7; | P53; | IGFBP2; | | | |
| | ECADHERIN; | PAI1; | SYK; | | | |
| | EEF2; | MYH11; | RAB25 | | | |
| | EGFR_pY1068; | NDRG1_pT346; | | | | |
| | HER2; | RICTOR; | | | | |
| | INPP4B; | TIGAR; | | | | |
| | MAPK_pT202Y204; | EPPK1 | | | | |
| | NFKBP65_pS536; | | | | | |
| | PTEN; | | | | | |
| | RB_pS807S811; | | | | | |
| | SRC_pY527; | | | | | |
| | ETS1; PDCD4; | | | | | |
| | PEA15_pS116; | | | | | |
| | RBM15; | | | | | |
| | TFRC | | | | | |
| TCGA_SARC | CASPASE7CLEAVEDD198; | X4EBP1_pT37T46; | X53BPLATM; | CK1T; CYCLINB1; | — | — |
| | PKCALPHA; | AKT_pS473; | IGFBP2; | FIBRONECTIN; | | |
| | RAD51; | AKT_pT308; | P70S6K1; | PAI1; PAXILLIN; | | |
| | SYK; | AR; | XBP1 | SRC_pY416; | | |
| | ANNEXIN1; | CAVEOLIN1; | | SRC_pY527; | | |
| | PREX1 | CYCLINE1; | | MYOSINIIA_pS1943 | | |
| | | GAB2; | | | | |
| | | GSK3ALPHABETA_pS21S9; | | | | |
| | | MAPK_pT202Y204; | | | | |
| | | MEK1; | | | | |
| | | NFKBP65_pS536; | | | | |
| | | P38_pT180Y182; | | | | |
| | | YAP_pS127; | | | | |
| | | GSK3_pS9; | | | | |
| | | MYH11; | | | | |
| | | NDRG1_pT346; | | | | |
| | | RAB25; | | | | |
| | | RICTOR; | | | | |
| | | EPPK1; | | | | |
| | | P62LCKLIGAND; | | | | |
| | | P16INK4A | | | | |
| TCGA_SKCM | BCL2; CKIT; | CASPASE7CLEAVEDD198; | X4EBP1_pT37T46; | AKT; | — | — |
| | CAVEOLIN1; | HSP70; PAI1; | AKT_pT308; | AKTpS473; | | |
| | COLLAGENV1; | PKCALPHA; | ATM; EEF2; | GAB2; | | |
| | FIBRONFCTIN; | PKCALPHA_pS657; | EEF2K; | GSK3ALPHABETA_pS21S9; | | |
| | MYH11; | PTEN; | HER3; | MAPK_pT202Y204; | | |
| | BRD4 | NDRG1_pT346; | NFKBP65_pS536; | PAXILLIN; | | |
| | | ANNEXIN1; | GAPDH; | RAD50; | | |
| | | P16INK4A | TFRC; | RB_pS807S811; | | |
| | | | P62LCKLIGAND | S6_pS235S236; | | |
| | | | | S6_pS240S244; | | |
| | | | | SRC_pY527; | | |
| | | | | STAT5ALPHA; | | |
| | | | | FASN; | | |
| | | | | GSK3pS9; | | |
| | | | | DUSP4 | | |
| TCGA_STAD | MAPK_pT202Y204; | ASNS; | BETACATENTN; | CASPASE7CLEAVEDD198; | CAVEOLIN1; | ATM; |
| | P27; SRC_pY527; | CLAUDIN7; | CYCLINE1; | ERK2; | HSP70; | G6PD |
| | NDRG1_pT346; | CYCLINB1; | PAI1; | MYOSINILA_pS1943; | IGFBP2; | |
| | EPPK1 | EGFR_pY1068; | PAXILLIN; | TRANSGLUTAMINASE; | STAT5ALPHA; | |
| | | HER2; RAB25; | RAD50; | ERCC1 | CD20; ETS1; | |
| | | TIGAR; | RB_pS807S811; | | MYH11; | |
| | | TFRC; SMAC; | P16INK4A | | PEA15_pS116; | |
| | | DUSP4 | | | RICTOR | |
| TCGA_TGCT | CKIT; | CAVEOLIN1; | ASNS; | AR; | — | — |
| | CHK2; | HSP70; | CYCLINB1; | CASPASE7CLEAVEDD198; | | |
| | CHK2_pT68; | PKCALPHA_pS657; | HER3; IGFBP2; | PKCALPHA; | | |
| | EEF2K; | SRC_pY527; | PAT1; | STAT5ALPHA; | | |
| | GAB2; KU80; | YAP_pS127; | SRC_pY416; | SYK; NDRG1_pT346; | | |
| | MTOR; S6; | RICTOR; | FASN; | PKCPANBETAII_pS660 | | |
| | CYCLINE2; | XBP1 | EPPK1 | | | |
| | PDCD4; | | | | | |
| | PRDX1; | | | | | |
| | PREX1; | | | | | |
| | ADAR1; MSH2; | | | | | |
| | MSH6 | | | | | |
| TCGA_THCA | AKT_pS473; | STAT3_pY705; | BETACATENTN; | MYH11; | FIBRONECTIN; | — |
| | BCL2; | CDK1_pY15; | CAVEOLIN1; | SCDI | HSP70; | |
| | CKIT; CLAUDIN7; | PDL1 | ECADHERIN; | | RAD50; | |
| | COLLAGENVI; | | ERK2; | | RAB25; | |
| | EGFR_pY1068; | | P38_pT180Y182; | | ANNEXIN1; | |
| | HER2_pY1248; | | YAP_pS127; | | PREX1; SMAC; | |
| | LKB1; | | PDCD4; PKCPAN | | DUSP4 | |
| | MAPK_pT202Y204; | | BETAII_pS660; | | | |
| | S6_pS240S244; | | SHP2_pY542 | | | |
| | SRC_pY527; | | | | | |
| | BRCA2; | | | | | |
| | ETS1; | | | | | |
| | PEA15_pS116; | | | | | |
| | TIGAR | | | | | |
| TCGA_THYM | CAVEOLIN1; | CK1T; | X4EBP1_pT37T46; | — | — | — |
| | CD49B; | CASPASE7CLEAVEDD198; | CYCLINB1; | | | |
| | DVL3; | EEF2K; | GAB2; | | | |
| | ECADHERIN; | STAT5ALPHA; | GATA3; | | | |
| | MAPK_pT202Y204; | XBP1 | GSK3ALPHABETA_pS21S9; | | | |
| | PAXILLIN; | | LCK; | | | |
| | SRC_pY527; | | NFKBP65_pS536; | | | |
| | NDRG1_pT346; | | P38_pT180Y182; | | | |
| | PDCD4; | | PCNA; | | | |
| | EPPK1; | | RB_pS8078811; | | | |
| | P62LCKLIGAND | | SMAD1; | | | |
| | | | SRC_pY416; | | | |
| | | | STATHMIN; | | | |
| | | | ETS1; | | | |
| | | | GSK3_pS9; | | | |
| | | | RBM15; | | | |
| | | | MSH2; | | | |
| | | | MSH6 | | | |
| TCGA_UCEC | AKT_pS473; | ACC1; | AMPKALPHA_pT172; | — | — | — |
| | AKT_pT308; | BETACATENIN; | ASNS; | | | |
| | AR; BCL2; | ECADHERIN; | CLAUDIN7; | | | |
| | CASPASE7CLEAVEDD198; | EEF2; | CYCLINB1; | | | |
| | CAVEOLIN1; | GAPDH; | CYCLINE1; | | | |
| | ERALPHA; | PEA15_pS116; | HER2; | | | |
| | ERALPHA_pS118; | TFRC; | IGFBP2; P53; | | | |
| | GSK3ALPHABETA_pS21S9; | EPPK1; | RB_pS807S811 | | | |
| | MAPK_pT202Y204; | ACETYLATUBULINLYS40; | | | | |
| | P38_pT180Y182; | P16INK4A | | | | |
| | PKCALPHA; | | | | | |
| | PKCALPHA_pS657; | | | | | |
| | SYK; GSK3_pS9; | | | | | |
| | MYH11; | | | | | |
| | NDRG1_pT346; | | | | | |
| | PDCD4; | | | | | |
| | RICTOR; | | | | | |
| | TRANSGLUTAMINASE; | | | | | |
| | ANNEXINRSLC1A5 | | | | | |
| TCGA_UCS | X4EBP1_pT37T46; | AKT_pS473; | — | — | — | — |
| | X53BP1; | CASPASE7CLEAVEDD198; | | | | |
| | ASNS; | GSK3ALPHABETA_pS21S9; | | | | |
| | ATM; | NFKBP65_pS536; | | | | |
| | FTBRONECTIN; | P38_pT180Y182; | | | | |
| | GAB2; HER2; | PAI1; | | | | |
| | IGFBP2; | PAXILLIN; | | | | |
| | P70S6K1; | RAD51; | | | | |
| | RAD50; | NDRG1_pT346 | | | | |
| | S6; SRC_pY527; | | | | | |
| | HEREGULIN; | | | | | |
| | XBP1; | | | | | |
| | ACETYLATUBULINLYS40; | | | | | |
| | MSH2; | | | | | |
| | MSH6; | | | | | |
| | BRD4; | | | | | |
| | P16INK4A; | | | | | |
| | PDL1 | | | | | |
| TCGA_UVM | EEF2K; | ACC_pS79; | X4EBP1_pT37T46; | — | — | — |
| | GSK3ALPHABETA_pS21S9; | ACC1; | BETACATENIN; | | | |
| | HER3; P38_pT180Y182; | AMPKALPHA_pT172; | ECADHERIN; | | | |
| | RAD51; SRC_pY527; | ATM; | GAB2; | | | |
| | YAP_pS127; BAP1C4; | BAK; | PAXILLIN; | | | |
| | GSK3_pS9; NDRG1_pT346; | CKIT; INPP4B; | ACETYLATUBULINLYS40; | | | |
| | P21; PDCD4; | LCK; | ADAR1; | | | |
| | TUBERIN_pT1462; | NFKBP65_pS536; | CDK1_pY15 | | | |
| | CASPASE8; | PKCALPHA; | | | | |
| | DUSP4; | PKCALPHA_pS657; | | | | |
| | ERCC5 | PTEN; | | | | |
| | | SRC_pY416; | | | | |
| | | RBM15; | | | | |
| | | P62LCKLIGAND; | | | | |
| | | PREX1 | | | | |

1. A method for determining an aryl hydrocarbon receptor (AHR) activation signature for a condition, comprising:
(a) providing at least two biological samples of the condition, wherein the at least two biological samples represent at least two different outcomes for the condition;
(b) detecting a biological state of each of the AHR biomarkers of Table 1 for the at least two biological samples;
(c) categorizing the AHR biomarkers into at least two groups based on the change of biological state of each marker compared to a control;
(d) categorizing the at least two groups into at least two subgroups based on at least one functional outcome of AHR signaling; and
(e) designating the markers in the at least two subgroups that correlate with the at least two different outcomes as the AHR activation signature for the condition.

2. The method of claim 1, wherein the biological state is RNA expression.

3. The method of claim 2, further comprising:
(f) detecting a second biological state of at least one biomarker for the at least two samples, wherein the second biological state is selected from the group consisting of mutation state, methylation state, copy number, protein expression, metabolite abundance, and enzyme activity;
(g) correlating the second biological state of the at least one biomarker with the least two subgroups that correlate with the at least two different outcomes; and
(h) designating the at least one biomarker as an alternative AHR activation signature for the condition if the second biological state of the at least one biomarker correlates with the at least two subgroups that correlate with the at least two different outcomes.

4. The method of claim 1, wherein the at least one functional outcome of AHR signaling is selected from the group consisting of angiogenesis, drug metabolism, external stress response, hemopoiesis, lipid metabolism, cell motility, and immune modulation.

5. The method of claim 4, wherein the at least one functional outcome of AHR signaling comprises angiogenesis, drug metabolism, external stress response, hemopoiesis, lipid metabolism, cell motility, or immune modulation.

6. The method of claim 1, wherein the categorizing in step (c) comprises grouping together AHR biomarkers that display at least 1.5 absolute fold upregulation or at least 0.67 absolute fold down-regulation in the biological state.

7. The method of claim 1, wherein the AHR activation signature comprises about 5, about 10, about 20, about 30 of the AHR biomarkers according to Table 1 or at least 10%, at least 20%, at least 30%, at least 40%, at least 50%, at least 60%, at least 70%, at least 80% or at least 90% or more or all of the AHR biomarkers according to Table 1.

8. The method of claim 1, wherein the categorizing steps are achieved by supervised clustering.

9. The method of claim 1, wherein the categorizing steps are achieved by unsupervised clustering.

10. The method of claim 1, wherein the biological sample is selected from the group consisting of biological fluids comprising biomarkers, cells, tissues, and cell lines.

11. The method of claim 10, wherein the biological sample is selected from primary cells, induced pluripotent cells (IPCs), hybridomas, recombinant cells, whole blood, stem cells, cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cell types thereof.

12. The method of claim 1, wherein the condition is selected from cancer, diabetes, autoimmune disorder, degenerative disorder, inflammation, infection, drug treatment, chemical exposure, biological stress, mechanical stress, and environmental stress.

13. A method comprising
(a) obtaining a biological sample from a subject;
(b) determining, in the biological sample, a biological state of each aryl hydrocarbon receptor (AHR) biomarker of an AHR activation signature, wherein the AHR activation signature is specific for a condition and comprises a subset of AHR biomarkers from Table 1;
(c) performing clustering of the AHR biomarkers based on the biological state of each AHR biomarker; and
(d) determining the AHR activation state of biological sample based on the clustering.

14.-26. (canceled)

27. The method of claim 13, further comprising treating the subject with an AHR signaling modulator.

28. The method of claim 27, wherein the AHR signaling modulator is selected from the group consisting of a 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, and a 2-heteroaryl-3-oxo-2,3-dihydro-4-carboxamide compound.

29. A method comprising:
(a) treating a cell with a compound;
(b) determining, in the cell, a biological state of each aryl hydrocarbon receptor (AHR) biomarker of an AHR activation signature, wherein the AHR activation signature is specific for a condition and comprises a subset of AHR biomarkers from Table 1;
(c) determining, in a control cell, the biological state of each AHR biomarker of the AHR activation signature;
(d) comparing the biological state from step (b) to the biological state from step (c);
(e) categorizing the compound based on the comparing step (d).

30. The method of claim 29, wherein the compound is an inhibitor of AHR signaling when the biological state from step (b) is less than the biological state from step (c), and the compound is an activator of AHR signaling when the biological state from step (b) is less than the biological state from step (c).

31-40. (canceled)

41. A processor programmed to perform:
(i) comparing biological states of aryl hydrocarbon receptor (AHR) biomarkers from at least two samples with known outcomes with biological states of the AHR biomarkers from a control sample;
(ii) categorizing the at least two samples into at least two groups based on the comparison in step (i);
(iii) categorizing the result of step (ii) into at least two subgroups based on at least one functional outcome; and
(iv) identifying AHR biomarkers that correlate with the known outcomes.

42.-48. (canceled)

49. A computer-readable storage device, comprising instructions to perform:
(i) comparing biological states of aryl hydrocarbon receptor (AHR) biomarkers from at least two samples with known outcomes with biological states of the AHR biomarkers from a control sample;
(ii) categorizing the at least two samples into at least two groups based on the comparison in step (i);
(iii) categorizing the result of step (ii) into at least two subgroups based on at least one functional outcome; and
(iv) identifying AHR biomarkers that correlate with the known outcomes.

50.-56. (canceled)

57. A processor programmed to perform:
(i) comparing biological states of aryl hydrocarbon receptor (AHR) biomarkers of an AHR activation signature from a sample with biological states of the AHR biomarkers of the AHR activation signature from a control sample, wherein the AHR activation signature is specific for a condition;
(ii) categorizing the sample into a group based on the comparison in step (i);
(iii) categorizing the result of step (ii) into a subgroup based on at least one functional outcome; and
(iv) determining AHR activation state of the sample.

58.-64. (canceled)

65. A computer-readable storage device, comprising instructions to perform:
(i) comparing biological states of aryl hydrocarbon receptor (AHR) biomarkers of an AHR activation signature from a sample with biological states of the AHR biomarkers of the AHR activation signature from a control sample, wherein the AHR activation signature is specific for a condition;
(ii) categorizing the sample into a group based on the comparison in step (i);
(iii) categorizing the result of step (ii) into a subgroup based on at least one functional outcome; and
(iv) determining AHR activation state of the sample.

66.-72. (canceled)
